SEQUENCE LISTING (1 sequence)

SEQ ID NO: 1 - protein, 18 aa, Artificial Sequence
  Artificial peptide sequence
VAPEEHPTLLTEAPLNPK

(19) United States
(12) Patent Application Publication
(10) Pub. No.: US 2019/0133985 A1
(43) Pub. Date: May 9, 2019

(54) COMPOSITIONS AND METHODS FOR USE OF EFLORNITHINE AND DERIVATIVES AND ANALOGS THEREOF TO TREAT CANCERS, INCLUDING GLIOMAS

(71) Applicant: Orbus Therapeutics, Inc., Palo Alto, CA (US)

(72) Inventor: Victor A. LEVIN, Palo Alto, CA (US)

(21) Appl. No.: 16/169,642

(22) Filed: Oct. 24, 2018

Related U.S. Application Data
(63) Continuation-in-part of application No. 15/218,149, filed on Jul. 25, 2016.
(60) Provisional application No. 62/312,623, filed on Mar. 24, 2016.

(51) Int. Cl. A61K 31/198; A61K 31/175; A61K 31/221; A61K 31/133; A61K 45/06; A61P 35/00

(52) U.S. Cl. CPC A61K 31/198; A61K 31/175; A61K 31/221; A61K 9/0053; A61K 45/06; A61P 35/00; A61K 31/133

(57) ABSTRACT

Eflornithine is an agent that can be used to treat glioma, especially glioma of WHO Grade II or Grade III such as anaplastic glioma. Eflornithine can suppress or prevent mutations in glioma which can cause the glioma to progress to a higher grade. Compositions and methods can include eflornithine or a derivative or analog of eflornithine, together with other agents such as conventional anti-neoplastic agents for treatment of glioma, inhibitors of polyamine transport, polyamine analogs, or S-adenosylmethionine decarboxylase inhibitors. Eflornithine or derivatives or analogs thereof can also be used to treat a range of non-glioma malignancies, including both malignancies of the central nervous system and other malignancies.

### CROSS-REFERENCES TO RELATED APPLICATIONS

This application is a continuation-in-part and claims the benefit of U.S. patent application Ser. No. 15/218,149, by Victor A. Levin, M.D., entitled “Compositions and Methods for Use of Eflornithine and Derivatives and Analogs Thereof to Treat Cancers, Including Gliomas,” filed Jul. 25, 2016, which, in turn, claimed the benefit of U.S. Provisional Application Ser. No. 62/312,623, by Victor A. Levin, M.D., entitled “Compositions and Methods for Use of Eflornithine and Derivatives and Analogs Thereof to Treat Cancers, Including Gliomas, filed Mar. 24, 2016. The contents of both of these applications are incorporated herein by this reference.

### FIELD OF THE INVENTION

This invention is directed to compositions and methods for the use of eflornithine and derivatives and analogs thereof to treat cancers, including gliomas, including the use of eflornithine (“DFMO”), whose full chemical name is D,L-2-(difluoromethyl) ornithine monohydrochloride monohydrate, and derivatives and analogs thereof to treat cancers together with other anti-neoplastic therapeutic agents, either simultaneously or sequentially. This invention is particularly directed to compositions and methods for decreasing the frequency of mutations in cancers induced by alkylating agents such as temozolomide (“TMZ”) in order to slow down progression or transformation of the disease and thus improve survival.

### BACKGROUND OF THE INVENTION

Glioma is one of the most common and serious forms of brain tumor. Gliomas are classified by cell type, by grade, and by location. Gliomas are generally named according to the specific type of cell with which they share histological features. These are not necessarily the cell types from which the glioma originated. The main types of glioma are: ependyoma (ependymal cells), astrocytoma (astrocytes), oligodendroglioma (oligodendrocytes), brainstem glioma (astrocytes), optic nerve glioma (cells in or around the optic nerve), and mixed glioma (cells from different types of glia). Gliomas are further characterized according to their grade, generally stated according to the WHO classification. Grade I is the lowest grade and are generally not infiltrative and have the best prognosis, and Grade I gliomas are generally considered benign. Some grade I glioma are subependymoma and juvenile pyelocytic astrocytoma (JPA). Grade II of the WHO classification is the next lowest grade. Gliomas of Grade II are well-differentiated and infiltrative but not anaplastic in appearance. Although these grade II tumors are generally associated with a favorable prognosis, they can recur and/or progress with an increase in tumor grade, and thus, in severity, over time. High-grade gliomas, Grades III and IV in the WHO classification, are undifferentiated or anaplastic and are clearly malignant. These grades carry the worst prognosis. Gliomas can also be classified according to their location, specifically whether they are above or below a membrane in the brain, the tentorium. The tentorium separates the cerebrum from the cerebellum. Supratentorial gliomas are more common in adults, while infratentorial gliomas are more common in children.

The symptoms of glioma generally depend on which part of the central nervous system is affected. Gliomas in the brain can cause headaches, vomiting, seizures, focal weakness, problems forming new memories, problems with speech, and cranial nerve disorders as a result of tumor growth. Gliomas of the optic nerve can cause visual disturbances or vision loss. Gliomas of the spinal cord can cause pain, weakness, or numbness in one or more extremities. Generally, gliomas do not metastasize through the bloodstream, but can spread through the cerebrospinal fluid and cause metastases in the spinal cord.

The exact causes of gliomas are not known. Certain hereditary genetic disorders such as type 1 or type 2 neurofibromatosis or tuberous sclerosis can predispose to their development. A number of oncogenes can be involved in glioma initiation and development. Many gliomas are infected with cytomegalovirus, which can accelerate their development. Germ-line (inherited) polymorphisms of the DNA repair genes ERCC1, ERCC2 (XPD) and XRCC1 can increase the risk of glioma. This indicates that altered or deficient repair of DNA damage can contribute to the formation of gliomas. Excess DNA damage can give rise to mutations through translesion synthesis. Furthermore, incomplete DNA repair can give rise to epigenetic alterations or epimutations. Such mutations and epimutations may provide a cell with a proliferative advantage which can then, by a process of natural selection, lead to progression to cancer. Epigenetic repression of DNA repair genes is often found in progression to sporadic glioblastoma. For instance, methylation of the DNA repair gene MGMT promoter was observed in a substantial fraction of glioblastomas. In addition, in some glioblastomas, the MGMT protein is deficient due to another type of epigenetic alteration. MGMT protein expression may also be reduced due to increased levels of a microRNA that inhibits the ability of the MGMT messenger RNA to produce the MGMT protein. It was found that, in glioblastomas without methylated MGMT promoters, that the level of microRNA miR-181d is inversely correlated with protein expression of MGMT and that the direct target of miR-181d is the MGMT mRNA 3′ UTR. Epigenetic reductions in expression of another DNA repair protein, ERCC1, were found in many gliomas; in some cases, the reduction was due to reduced or absent ERCC1 protein expression. In other cases, the reduction was due to methylation of the ERCC1 promoter. In a small number of cases, the reduction has been attributed to epigenetic alterations in microRNAs that affect ERCC1 expression. When expression of DNA repair genes is reduced, DNA damage can accumulate in cells at increased levels. In gliomas, mutations frequently occur in the isocitrate dehydrogenase genes IDH1 and IDH2. These mutations may result in production of an excess metabolic intermediate, 2-hydroxyglutarate, which binds to catalytic sites in key enzymes that are important in altering histone and DNA promoter methylation. This may result in a DNA CpG island methylator phenotype (CIMP) that can cause promoter hypermethylation and concomitant silencing of tumor suppressor genes such as DNA repair genes MGMT and ERCC1. Additionally, mutations in IDH1 and IDH2 may cause increased oxidative stress and thus initiate increased oxidative damage to DNA.

Several acquired genetic mutations are commonly found in gliomas, including mutations in p53 and PTEN; the gene encoding PTEN may also be lost. These mutations can lead to overexpression of EGFR. However, hypermutation associated with gliomas is not confined to specific locations.

High-grade gliomas are highly vascular tumors and have a tendency to infiltrate the CNS. They also may have extensive areas of necrosis and hypoxia. As a rule, high-grade gliomas almost always grow back even after complete surgical excision, so are commonly called recurrent high-grade glioma or glioblastoma. In contrast, lower-grade gliomas typically grow relatively slowly and can be followed without the need for aggressive treatment unless they grow or cause symptoms.

Treatment for gliomas depends on the location, the cell type, and the grade of malignancy. A combined approach, including surgical resection, radiotherapy, and chemotherapy, is frequently employed. One therapeutic agent frequently employed is temozolomide, which can cross the blood-brain barrier and is frequently used in treatment of higher-grade gliomas. The angiogenic blocker bevacizumab, a monoclonal antibody, is also frequently used. However, there is increasing evidence that the use of temozolomide may itself induce mutations and worsen prognosis in a significant fraction of patients (B. E. Johnson et al., “Mutational Analysis Reveals the Origin and Therapy-Driven Evolution of Recurrent Glioma,” Science 343: 189-193 (2014)). The potentially mutagenic effect of temozolomide must be taken into account in planning a course of treatment for glioma. Other anti-neoplastic agents are also potentially mutagenic, and this also needs to be taken into account in planning a course of treatment for glioma.

Gliomas are rarely curable. The prognosis for patients with high-grade gliomas is generally poor, and is especially so for older patients. Of 10,000 Americans diagnosed each year with malignant gliomas and based on CBTRUS (table 23, 2015 edition), about 57% are alive one year after diagnosis, 41% after two years, and only 31% at five years. Those with anaplastic astrocytoma have about 44% alive at two years and 28% at five years. Glioblastoma multiforme has a worse prognosis with a 37% one year survival and 15% two year survival after diagnosis. For low-grade gliomas, the prognosis is somewhat more optimistic, but even such patients have a far higher death rate than does the general population when age is taken into account.

Therefore, there is a substantial need for an improved treatment for gliomas. In addition, there is a particular need to provide treatments that can avoid or counteract the potentially mutagenic effect of the frequently-used antineoplastic alkylating drugs such as temozolomide. Additionally, there is a substantial need for treatments that can block or reverse immunosuppression of tumor cells. As detailed below, the principles of treatment provided in the present invention can also be applied to malignancies in general, including malignancies other than gliomas or other than malignancies of the central nervous system, as cancer is typically characterized by mutation of the neoplastic cells.

### SUMMARY OF THE INVENTION

The present invention provides a new therapeutic modality for the treatment of glioma and other malignancies.

One aspect of the present invention is a method for the treatment of glioma comprising the step of administering a therapeutically effective quantity of eflornithine or a derivative or analog thereof to a subject with glioma in order to reduce the rate of hypermutation of the glioma to reduce the progression or increasing grade of severity of the glioma caused by alkylating therapy exposure. Typically, a gene undergoing hypermutation is at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16. More typically, a gene undergoing hypermutation is at least one gene selected from the group consisting of H1FNT, RFX1, DHX36, MBD2, FRK, PEG3, NDC80, ABCA6, DNLZ, MUC12, PDCD6IP, and GSC2.

The subject with glioma can be currently treated with an alkylating agent. In one alternative, the alkylating agent is selected from the group consisting of temozolomide and lomustine. The alkylating agent can be another alkylating agent known in the art. Alternatively, the subject with glioma can have previously been treated with an alkylating agent.

In one alternative, the eflornithine or derivative or analog thereof is eflornithine, such as a racemic mixture of D-eflornithine and L-eflornithine, D-eflornithine, or L-eflornithine. In another alternative, the eflornithine or derivative or analog thereof is a derivative or analog of eflornithine.

The method can further comprise the step of detecting the mutation. The mutation can be detected by DNA sequencing or by another method known in the art.

In one alternative, the eflornithine or derivative or analog thereof is administered orally or by injection. In another alternative, the eflornithine or derivative or analog thereof is administered together with or adjuvant to radiotherapy.

Typically, the glioma is characterized by one or more of the following characteristics:

(1) the glioma was previously treated with radiation therapy or radiation therapy with temozolomide chemotherapy and adjuvant alkylator therapy, which can be concurrent, adjuvant, or sequential exposure, and is recurrent/refractory anaplastic glioma;

(2) the glioma has a mutation in one or more genes selected from the group consisting of IDH1, IDH2, TP53, PTEN, ATRX, BRAF, CDKEN2A, SMARCA4, and PIK3;

(3) the glioma has the promoter for MGMT methylated; and

(4) the glioma has a mutation in at least one other gene that affects proliferation, survival, or resistance to chemotherapy.

In one alternative, the eflornithine or derivative or analog thereof is administered together with a therapeutically effective quantity of one or more conventional antineoplastic agents used for the treatment of glioma, wherein the one or more conventional antineoplastic agents are selected from the group consisting of: alkylating agents, platinum-containing agents that damage DNA, antimetabolites, anti-angiogenic agents, EGFR inhibitors, topoisomerase inhibitors, and tyrosine kinase inhibitors. The eflornithine or derivative or analog thereof can also be administered with other additional agents or with an immunomodulatory agent.

Another aspect of the present invention is a method for the treatment of a malignancy selected from the group consisting of a non-glioma central nervous system malignancy, colorectal cancer, leukemia, lymphoma, pancreatic cancer, liver cancer, and stomach cancer comprising the step of administering a therapeutically effective quantity of eflornithine or a derivative or analog thereof to a subject with the malignancy in order to reduce the rate of hypermutation of the malignancy to reduce the progression of the malignancy, wherein the reduction of the rate of hypermutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16.

The characteristics of the method are generally similar to the methods described above with respect to the treatment of glioma, in terms of the eflornithine or derivative or analog used, the present or prior treatment with an alkylating agent, the routes of administration, and the use with other agents.

Another aspect of the present invention is a pharmaceutical composition for the treatment of glioma comprising:

(1) a therapeutically effective quantity of eflornithine or a derivative or analog thereof, wherein the therapeutically effective quantity of eflornithine or the derivative or analog thereof reduces the rate of mutation of the glioma to reduce the progression of the glioma, wherein the reduction of the rate of mutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16; and

(2) a pharmaceutically acceptable excipient.

The eflornithine or derivative or analog thereof is as described above. The composition can be formulated for administration by injection or oral administration. The composition can further comprise a therapeutically effective quantity of one or more conventional antineoplastic agents used for the treatment of glioma, wherein the one or more conventional antineoplastic agents used for the treatment of glioma are selected from the group consisting of alkylating agents, platinum-containing agents that damage DNA, antimetabolites, anti-angiogenic agents, EGFR inhibitors, topoisomerase inhibitors, and tyrosine kinase inhibitors. Alternatively, the composition can further comprise one or more additional agents.

Typically, the pharmaceutically acceptable excipient is selected from the group consisting of:
(i) a liquid carrier;
(ii) an isotonic agent;
(iii) a wetting or emulsifying agent;
(iv) a preservative;
(v) a buffer;
(vi) an acidifying agent;
(vii) an antioxidant;
(viii) an alkalinizing agent;
(ix) a carrying agent;
(x) a chelating agent;
(xi) a coloring agent;
(xii) a complexing agent;
(xiii) a solvent;
(xiv) a suspending and/or viscosity-increasing agent;
(xv) a flavor, perfume, or sweetening agent;
(xvi) an oil;
(xvii) a penetration enhancer;
(xviii) a polymer;
(xix) a stiffening agent;
(xx) a protein;
(xxi) a carbohydrate;
(xxii) a bulking agent; and
(xxiii) a lubricating agent.

Yet another aspect of the present invention is a pharmaceutical composition for the treatment of a malignancy selected from the group consisting of a non-glioma central nervous system malignancy, colorectal cancer, leukemia, lymphoma, pancreatic cancer, liver cancer, and stomach cancer comprising:

(1) a therapeutically effective quantity of eflornithine or a derivative or analog thereof, wherein the therapeutically effective quantity of eflornithine or the derivative or analog thereof reduces the rate of mutation of the malignancy to reduce the progression of the malignancy, wherein the reduction of the rate of mutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16; and

(2) a pharmaceutically acceptable excipient.

The pharmaceutical compositions described above, either for treatment of glioma or for the treatment of another malignancy, can further include a therapeutically effective quantity of an alkylating agent.

Yet another aspect of the invention is a kit, comprising, separately packaged:

(1) a therapeutically effective quantity of eflornithine or a derivative or analog thereof, wherein the quantity of the eflornithine or the derivative or analog thereof is a therapeutically effective quantity for treatment of a glioma such that the rate of mutation of the glioma is reduced to reduce the progression of the glioma, wherein the reduction of the rate of mutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16;

(2) a therapeutically effective quantity of an alkylating agent for treatment of the glioma; and

(3) instructions for use of the kit.

Typically, the eflornithine or derivative or analog thereof reduces the rate of mutation of the glioma associated with the administration of an alkylating agent. The alkylating agent can be temozolomide or another conventionally used alkylating agent, such as lomustine. While, in general, alkylating agents are known to be mutagenic, the degree to which they produce mutation varies among alkylating agents, with monofunctional alkylating agents (such as temozolomide) producing more mutation than bifunctional DNA cross linking alkylating agents (such as lomustine).

In one alternative, the glioma was previously treated with radiation therapy or radiation and temozolomide chemotherapy and adjuvant alkylator therapy, which can be concurrent, adjuvant, or sequential, and is recurrent/refractory anaplastic glioma. The use of radiation therapy is conventional for glioma (M. D. Prados et al., “Phase III Trial of Accelerated Hyperfractionation with or without Difluromethylornithine (DFMO) Versus Standard Fractionated Radiotherapy with or without DFMO for Newly Diagnosed Patients with Glioblastoma Multiforme,” Int. J. Rad. Oncol. Biol. Phys. 49: 71-77 (2001); M. S. Berger et al., “Primary Cerebral Tumors” in Cancer in the Nervous System (V. A. Levin, Oxford University Press, New York, 2002), pp. 75-148)).

The glioma can have the promoter for MGMT methylated. The glioma can also have one or more other mutations that affect proliferation, survival, or resistance to chemotherapy. One form of glioma that can be treated is astrocytoma.

### BRIEF DESCRIPTION OF THE DRAWINGS

These and other features, aspects, and advantages of the present invention will become better understood with reference to the following description, appended claims, and accompanying drawings where:

FIG. 1 is a series of graphs showing the effect of temozolomide (“TMZ”) and eflornithine on the mutation frequency in the glioblastoma cell line model U87MG for the temozolomide concentration EC-10 and the eflornithine (“DFMO”) concentration of 50 μM. FIG. 1A shows the effect of TMZ on U87MG cells compared to untreated cells on Day 3. FIGS. 1B and 1C show the results of mutation frequency measured in cells after subsequent application of eflornithine on days 7 and 14, respectively. FIG. 1D summarizes the data for all time points to demonstrate the overall effect of TMZ and subsequent DFMO treatment on mutation frequency of U87MG cells (Example 2).

FIG. 2 is a series of graphs showing the effect of temozolomide and eflornithine on the mutation frequency in the glioblastoma cell line model U87MG for the temozolomide concentration EC-20 and the eflornithine concentration of 100 μM. FIG. 2A shows the effect of TMZ on U87MG cells compared to untreated cells on Day 3. FIGS. 2B and 2C show the results of mutation frequency measured in cells after subsequent application of eflornithine on days 7 and 14, respectively. FIG. 2D summarizes the data for all time points to demonstrate the overall effect of TMZ and subsequent eflornithine treatment on mutation frequency of U87MG cells (Example 3).

FIG. 3 is a series of graphs showing the effect of temozolomide and eflornithine on the mutation frequency in the glioblastoma cell line model U87MG for the temozolomide concentration EC-50 and the eflornithine concentration of 200 μM. FIG. 3A shows the effect of TMZ on U87MG cells compared to untreated cells on Day 3. FIGS. 3B and 3C show the results of mutation frequency measured in cells after subsequent application of eflornithine on days 7 and 14, respectively. FIG. 3D summarizes the data for all time points to demonstrate the overall effect of TMZ and subsequent eflornithine treatment on mutation frequency of U87MG cells (Example 4).

FIG. 4 is a graph showing the results of the effect of temozolomide and eflornithine on the mutation frequency of a specific gene, TP53BP1, in U87MG cells (Example 6).

FIG. 5 is a graph showing the results of the effect of temozolomide and eflornithine on the mutation frequency of a specific gene, ADAM32, in U87MG cells (Example 7).

FIG. 6 is a graph showing the results of the effect of temozolomide and eflornithine on the mutation frequency of a specific gene, GPR116, in U87MG cells (Example 8).

FIG. 7 is a graph showing the results of the effect of temozolomide and eflornithine on the mutation frequency of a specific gene, MUC16, in U87MG cells (Example 9).

### DETAILED DESCRIPTION OF THE INVENTION

In general, this invention is directed to the treatment of temozolomide recurrent/refractory anaplastic astrocytoma patients with eflornithine alone or in combination with lomustine and other chemotherapy agents. In addition to eflornithine, derivatives or analogs of eflornithine can be used, as detailed further below. However, in addition to anaplastic astrocytoma or other forms of glioma, as detailed further below, methods and compositions according to the present invention can be used to treat other malignancies in addition to glioma.

Neutral non-proteinogenic amino acids that are also AA T6 transporter substrates (such as eflornithine) can extend life of cancer patients by inhibiting progression of DNA mutations caused by chemotherapy agents.

The basis of the invention, in general, is as follows: (1) chemotherapy agents cause DNA mutations; (2) eflornithine interrupts cellular proliferation and differentiation; and (3) by interrupting cellular proliferation and differentiation in cancer cells, eflornithine inhibits mutations and thus constrains progression of cancer. Therefore, since alkylating chemotherapy agents cause DNA mutations, use of eflornithine with these agents inhibits those mutations and thus improves survival of cancer patients. The use of eflornithine therefore provides a survival advantage for treatment with eflornithine over conventional treatment with lomustine or treatment with procarbazine, lomustine, and vincristine (based on treatment protocols described in V. A. Levin et al., “Treatment of Recurrent Gliomas with Eflornithine,” J. Natl. Cancer Inst. 84: 1432-1437 (1992) (eflornithine); V. A. Levin et al., “Phase III Randomized Study of Postradiotherapy Chemotherapy with Combination Alpha-Difluoromethylornithine-PCV Versus PCV for Anaplastic Gliomas,” Clin. Cancer Res. 9: 981-990 (2003) (eflornithine); M. C. Chamberlain, “Salvage Therapy with Lomustine for Temozolomide Refractory Recurrent Anaplastic Astrocytoma: A Retrospective Study,” J. Neurooncol. 122: 329-338 (2015) (lomustine). Additionally, tumors may undergo spontaneous mutations over time that also might be reduced by eflornithine.

This is particularly significant because, recently, tumors in glioma patients treated with temozolomide (a monofunctional DNA alkylating agent) have been shown to follow a different evolutionary path to high-grade glioma (Johnson et al., Science 343: 189-193 (2014)). These tumors were hypermutated as a result of temozolomide exposure, and thus have a different clonal evolution when compared to similar tumors that have progressed without exposure to temozolomide chemotherapy (Johnson et al. (2014), supra). It has also been shown that the degree of mutations related to glioma can change over the course of the disease (Mazor et al., Cancer Cell 28: 307-317 (2015)). In fact, inactivation of the DNA mismatch repair pathway has been shown to be associated with therapeutic resistance and it is now understood that temozolomide exposure can induce mutagenesis driving disease progression (van Thuijl et al., Acta Neuropathol. 129: 597-607 (2015)).

It is well understood that inhibiting polyamine biosynthesis such as by inhibiting ornithine decarboxylase reduces tumor cell proliferation (Gerner et al., Nature Rev. 4: 781-792 (2004)). It is also well documented that temozolomide causes increasing mutation frequency in cancer cells in situ and may be responsible for increased disease severity in post-temozolomide exposure patients whose tumors progress (Johnson et al. (2014), supra). Here a hypothesis is presented that eflornithine through its activity inhibiting ornithine decarboxylase may contribute to reducing the rate at which tumor cells mutate after temozolomide exposure. This was studied in monolayer culture using two known glioma primary cell lines available from ATCC. First, the effective concentration of temozolomide in monolayer culture by measuring the extent to which cells survive over time across a range of drug concentrations was defined. This enabled the identification of the EC-10, the EC-20, and the EC-50 for temozolomide in monolayer culture, wherein 10%, 20%, and 50% of the cells did not survive at defined concentrations of temozolomide over time. This was used to then evaluate the impact that eflornithine had when cells were sequentially treated with temozolomide and then eflornithine, mimicking human clinical exposure.

Use of the Catalogue of Somatic Mutations in Cancer (COSMIC) database and the GeneCards® Human Genome Database enabled the elucidation of the relationships between the presence of mutations in these genes and certain cancers. The effect on known nucleotide polymorphisms across all 22 human autosomes using a primary glioma cell line was chosen for further evaluation. Since this cell line comes from diseased tissue, it contains an inherent degree of mutational burden as defined by the presence of nucleotide polymorphisms across known cancer related genes. The frequency of nucleotide polymorphisms prior to and after temozolomide exposure was quantified, illustrating that there is a measurable increase in mutation frequency across hundreds of previously identified genes. When the cells were exposed to eflornithine, either concurrent to exposure to temozolomide or after exposure to temozolomide, the average mutation frequency decreased and, in some cases, returned to the level equal to the untreated control group (see Examples).

As the number of polymorphisms and the number of genes being analysed were both very large, it was determined that the somatic mutations could first be analysed in the aggregate (see Examples). A self-imposed threshold for defining what change in mutation frequency would be considered meaningful was defined to be equal to or greater to a 15% increase in mutation frequency, as measured by the frequency of nucleotide polymorphism in each analysed gene in temozolomide-treated cells compared to untreated controls. Several hundred somatic mutations were identified as having an increase in frequency greater than 15% across all temozolomide concentrations used in vitro.

For the purposes of describing the implications of the changing frequency of somatic mutations that result from temozolomide exposure followed by or occurring simultaneously with eflornithine exposure, rather than try to comprehensively assess all the genes with greater than 15% mutation frequency changes across all concentrations of temozolomide use, the sample set was reduced to a more manageable number to illustrate the biological and pathological relevance of these mutations. As a result, certain mutations have been highlighted that are known to be relevant to glioma (see Examples). Specifically, these gene examples have been highlighted in recent studies of hypermutation related to malignant glioma and temozolomide exposure (Johnson et al. (2014), supra; Mazor et al. (2015), supra; van Thuijl et al. (2015), supra). A description of each of these genes is shown in the Examples.

Eflornithine occurs in two enantiomeric forms: D-eflornithine and L-eflornithine. D-eflorninthine is shown in Formula (Ia), below. L-eflornithine is shown in Formula (Ib), below.

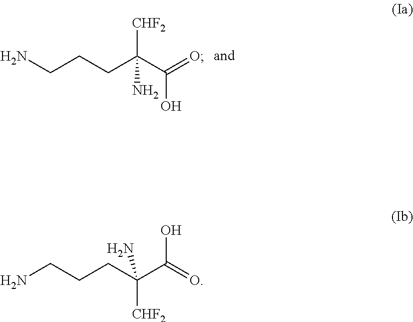 [SMILES: NCCC[C@](N)(C(=O)O)C(F)F ; NCCC[C@@](N)(C(=O)O)C(F)F]

Typically, eflornithine is administered as the racemic mixture of D-eflornithine and L-eflornithine. However, eflornithine can also be administered in a mixture in which the D-eflornithine is relatively enriched with respect to the Leflornithine, or in a pure or substantially pure preparation of D-eflornithine.

Eflornithine is a structural analog of the amino acid L-ornithine (shown below as Formula (II):

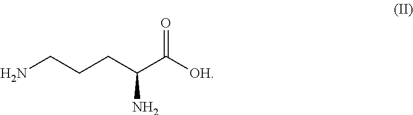

It is known that catalysis by ornithine decarboxylase (ODC) is the rate-limiting step in polyamine synthesis. The pathway for polyamine synthesis begins with L-ornithine. This natural amino acid, although not normally incorporated into proteins, is part of the urea cycle which metabolizes arginine to ornithine and urea. Ornithine is converted by ornithine decarboxylase (ODC) to putrescine and CO₂ and is considered to be the rate-limiting step in the production of polyamines. With the addition of propylamine donated from S-adenosylmethionine, putrescine is converted to spermidine. Spermidine is then converted to spermine by spermine synthetase, again in association with the decarboxylation of S-adenosylmethionine. Putrescine, spermidine and spermine represent the three major polyamines in mammalian tissues. Polyamines are found in animal tissues and microorganisms and are known to play an important role in cell growth and proliferation. Although the mechanism of the action of eflornithine in treating glioma is believed to involve primarily the prevention of induction of mutation in tumor cells, the effect of eflornithine on the synthesis of polyamines may play a secondary role. As detailed below, eflornithine or its derivatives or analogs may also have an effect on the immune system which enhances the effectiveness of immune surveillance.

A number of derivatives and analogs of eflornithine are known in the art, and are described further below.

Eflornithine is an irreversible inhibitor of the enzyme ornithine decarboxylase (ODC) and was originally developed as a treatment for trypanosomiasis (1-3). It has also been studied as a treatment for a variety of cancers (4).

Eflornithine can be administered either orally or by injection, such as intravenously or intraperitoneally. Other potential routes of administration of eflornithine are also known in the art.

While it has been established that the primary action of eflornithine is to inhibit ODC activity and, thereby, the production of putrescine from ornithine, its pleiotropic effect as an anticancer agent has not been fully realized or understood at this time. Many actions have been proposed to explain the effectiveness of eflornithine on tumor cells (4-6). It has been long-held dogma that since polyamines (putrescine, spermidine, and spermine) play essential roles in DNA and RNA function that inhibition of ODC would inhibit tumor growth, and possibly tumor cell migration. Eflornithine can also reduce the effect of chemical carcinogens on colonic, skin, and bladder tissues and cell lines and in clinical settings (4, 6).

However, over the past several years new insights have come forward that suggests a different antitumor activity for eflornithine against CNS gliomas than had previously been realized or suggested. The invention is directed to this new basis for antitumor activity. Specifically, we assert that a major anticancer benefit of eflornithine rests with its ability to modulate (downregulate) mutation in slowly growing infiltrative gliomas (WHO Grade II and III tumors). By reducing the occurrence and/or number of mutations, it is believed that tumor growth in the patient will cease and/or slow because the mutational-activated drivers of cancer progression and transformation to the more malignant glioblastoma (WHO Grade IV) will fail to occur or be less numerous over time.

A phase 3 randomized trial of adjuvant chemotherapy of eflornithine-PCV versus PCV (procarbazine/CCNU/vincristine) in anaplastic glioma patients (7) provides evidence for this hypothesis. That study showed that eflornithine-PCV chemotherapy produced a shift in the progression-free survival (PFS) hazard function compared to PCV chemotherapy that lasted about 1-1.5 years after the eflornithine-PCV stopped. From that point forward, the PFS and overall survival (OS) curves remained parallel, but did not cross, for over a decade (7,8). The hypothesis includes the probability that eflornithine protected against progression of anaplastic gliomas (especially anaplastic astrocytoma) to a more malignant phenotype, such as glioblastoma. Additional support comes from recent studies in neuroblastoma cells that found that eflornithine could increase two intracellular proteins, p21 and p27kip-1, and thereby arrest cell division between G1 and the initiation of mitosis (9,10).

As described above, treatment with eflornithine provides a substantial survival advantage over treatment with lomustine, particularly in cases of recurrent anaplastic glioma. However, the basis for this survival advantage and how it could be exploited in the most optimal manner, or exploited for cancers other than glioma, is not well understood.

Taken together, these two observations suggest that because eflornithine can be safely administered orally for 2 weeks every 3 weeks for years at a time, produce G1 arrest, and increase in intracellular p21 and p27kip-1 it is highly likely that eflornithine will reduce mutation-rates in glioma tumor cells in situ and, thereby, provide new and unexpected effects on the transformation of low- (WHO Grade II) and mid-grade (WHO Grade III) gliomas to glioblastoma (WHO Grade IV). This approach will, by its action, limit mutation and produce long-term survival gains for patients with these tumors as was shown in the clinical trial (7,8). It also suggests that treatment with eflornithine should continue for years in patients with low- and mid-grade gliomas. The increase in intracellular p21 and p27kip-1 induced by the administration of eflornithine is associated with the suppression of mutation by this agent, which has the clinical consequences of preventing or delaying the progression of the glioma to a higher grade. The survival advantage resulting from treatment with eflornithine is described above.

Additional results support this hypothesis (11). These results show the importance of mutation to transformation of low- and mid-grade gliomas to more malignant tumor grades. The premise of this study was that therapies for recurrent or progressive gliomas failed because the genomic alterations driving the growth of recurrences were distinct from those in the initial tumor. In this study, the exomes of 23 initial low-grade gliomas and recurrent tumors resected from the same patients were sequenced. It was found that the three genes most commonly mutated in Grade 2 glioma at initial diagnosis were: IDH1 in 100% (23/23), TP53 in 83% (19/23), and ATRX in 78% (18/23) in the cohort studied. The next most commonly mutated gene, SMARCA4, was identified in 13% (3/23) of the initial tumors in this cohort. They also found 13 additional genes that could be identified in 9% (2/23) of the cohort. Interestingly, in 43% of cass, at least half of the mutations in the initial tumor were undetected at tumor recurrence/progression, including driver mutations in TP53, ATRX, SMARCA4, and BRAF, suggesting that recurrent tumors may be seeded by cells derived from the initial tumor at a very early stage of their evolution. This emphasizes the importance of early treatment for these tumors. Of additional interest also was the observation that tumors from 6 of 10 patients treated with adjuvant temozolomide (TMZ) chemotherapy followed an alternative evolutionary path to high-grade glioma: these tumors showed hypermutation and harbored driver mutations in the RB and AKT-mTOR pathways that bore the signature of TMZ-induced mutagenesis. These studies extended earlier observations and studies of primary GBMs (12,13), unpaired recurrent tumors (14), and a cell culture model (15). In particular, tumors in glioma patients treated with temozolomide (a monofunctional DNA alkylating agent) have been shown to follow a different evolutionary path to high-grade glioma (Johnson et al., Science 343: 189-193 (2014)). These tumors were hypermutated as a result of temozolomide exposure and, thus, have a different clonal evolution when compared to similar tumors that have progressed without exposure to temozolomide chemotherapy. It has also been shown that the degree of mutations related to glioma can change over the course of the disease (Mazor et al., Cancer Cell 28: 307-317 (2015)). In fact, inactivation of the DNA mismatch repair pathway has been shown to be associated with therapeutic resistance and it is now understood that temozolomide exposure can induce mutagenesis driving disease progression (van Thuijl et al., Acta Neuropathol. 129: 597-607 (2015)).

It is well understood that inhibiting polyamine biosynthesis, such as by inhibiting the activity of ornithine decarboxylase, reduces tumor cell proliferation (Gerner et al., Nature Rev., 4: 781-792 (2004)). It is also well documented that temozolomide causes increasing mutation in cancer cells in situ and may be responsible for increased disease severity in post-temozolomide exposure patients whose tumors progress. The hypothesis presented herein is that eflornithine through its inhibition of ornithine decarboxylase may contribute to reducing the rate at which tumor cells mutate after temozolomide exposure. The Examples included herein illustrate this hypothesis in monolayer tissue culture using two known glioma primary cell lines available from ATCC. The effective concentration of temozolomide was in monolayer culture was first defined by measuring the extent to which cells survive over time across a range of drug concentration. This enabled the determination of EC10, EC20, and EC50 for temozolomide in monolayer culture, wherein 10%, 20% and 50% of cells did not survive at defined concentrations over time. This was used to then evaluate the impact that eflornithine had when cells were sequentially treated with temozolomide and then eflornithine, mimicking human clinical exposure.

Use of the Catalogue of Somatic Mutations in Cancer database (COSMIC) and the GeneCards® Human Genome Database enabled the elucidation of the relationships between the presence of mutation in these genes and certain cancers. Furthermore, the ability to quantify the effect on known nucleotide polymorphisms across all 22 human autosomes using a primary glioma cell line enabled the evaluation described herein. Since this cell line comes from diseased tissue, it contains an inherent degree of mutational burden as defined by the presence of nucleotide polymorphisms across known cancer-related genes. The frequency of nucleotide polymorphisms was quantified prior to and after temozolomide exposure illustrating that there is a measurable increase in mutation frequency across hundreds of previously identified genes related to cancer (see Examples). When the cells were exposed to eflornithine, either concurrent to temozolomide or after temozolomide, the average mutation frequency caused by temozolomide decreased and, in some cases returned to the level equal to the untreated control group (see Examples).

As the number of polymorphisms and number of genes being analyzed is very large, it was determined that the somatic mutations could first be analyzed in the aggregate (see Examples). A threshold was self-imposed for defining what the change in mutation frequency was to be considered meaningful; this threshold was set at equal to or greater than a 15% increase in mutation frequency, as measured by the frequency of nucleotide polymorphism in each analyzed gene, in temozolomide treated cells compared to untreated control. Several hundred somatic mutations were identified as having an increase in frequency greater than 15% across all three temozolomide concentrations used in vitro.

For the purposes of describing the implications of the changing frequency of somatic mutations that result from temozolomide exposure followed by eflornithine exposure, rather than try to comprehensively assess all the genes with greater than 15% mutation frequency changes across all concentrations of temozolomide use, this was reduced to a more manageable number to illustrate the biologic relevance of these mutations. As a result, mutations are highlighted that are known to be relevant to glioma (see Examples). Specifically, these gene examples have been highlighted in recent studies of hypermutation related to malignant glioma and temozolomide exposure; in many cases, these gene examples are known to be associated with such processes as cell proliferation, differentiation, or resistance to apoptosis. A description of each of these genes is shown in the Examples.

In addition to the genes shown in the Examples, mutations in other genes have been shown to be induced by temozolomide. For these genes, the rate of temozolomide-induced mutation is reduced by the subsequent administration of eflornithine. These genes play various roles in carcinogenesis or the progression of malignancies, including, but not necessarily limited to, glioma.

CACNA1B on chromosome 9 is Calcium Channel, Voltage-Dependent, N Type, Alpha 1B Subunit. The protein encoded by this gene is the pore-forming subunit of an N-type voltage-dependent calcium channel which controls neurotransmitter release from neurons. Mouse insertional mutagenesis experiments support CACNA1B as a cancer causing gene that affects intracellular processes.

DNLZ on chromosome 9 is DNL-Type Zinc Finger. This is a protein coding gene that encodes a DNA-binding protein gene of the zinc finger type that is involved in chaperone binding. Mouse insertional mutagenesis experiments support DNLZ as a cancer causing gene in colorectal and central nervous system cancers.

MUC12 on chromosome 7 is Mucin 12, Cell Surface Associated. This is a protein coding gene that encodes an integral membrane protein that is a member of the mucin family. It is a cell surface associated protein coding gene and encodes an integral membrane glycoprotein. Diseases associated with expression of MUC12 include colorectal cancer (when its expression is downregulated) and immune system disorders. Pathways related to MUC12 include the immune system and the HIV life cycle.

ICAM1 on chromosome 19 is Intercellular Adhesion Molecule 1. This gene encodes a cell surface glycoprotein which is typically expressed on endothelial cells and cells of the immune system. Diseases and conditions associated with ICAM1 include malaria and leukostasis. Cell adhesion molecules (CAMs) are a large family of transmembrane proteins that are involved in the binding of a cell to another cell or to the extracellular matrix. They have roles in cell proliferation, differentiation, motility, trafficking, apoptosis and tissue architecture. Mouse insertional mutagenesis experiments support ICAM1 as a cancer causing gene.

LRP1B on chromosome 2 is Low Density Lipoprotein Receptor-Related Protein 1B. This gene encodes a member of the low density lipoprotein (LDL) receptor family. Disruption of this gene has been reported in several types of cancer including endocervical carcinoma. Mouse insertional mutagenesis experiments support LRP1B as a cancer causing gene.

PCDHGC3 on chromosome 5 is Protocadherin Gamma Subfamily C, 3. This is a member of the protocadherin gamma gene cluster, one of three related clusters tandemly linked on chromosome 5. Neural cadherin-like cell adhesion proteins most likely play a critical role in the establishment and function of specific cell-cell connections in the brain. Alternative splicing has been described for the gamma cluster genes. The gene encoded by PCDHGC3 may be involved in the establishment and maintenance of specific neuronal connections in the brain. Mutations in PCDHGC3 associated with altered sensitivity to the RET tyrosine kinase inhibitor XMD15-27.

H1FNT is H1 Histone Family, Member N, Testes-Specific on chromosome 12. It encodes a nuclear protein that is a component of chromatin and is testis-specific. H1FNT is essential for nuclear formation in spermatozoa and is involved in replacement of histones with protamines during spermiogenesis; it is also involved in cellular differentiation and structuring. Mutations in H1 variants are believed to be associated with malignancies, such as by altering the association of the histones in which they occur with the DNA and affecting regulation of gene expression.

RFX1 is MHC Class II Regulatory Factor RFX1 on chromosome 19. This gene is a regulatory factor X (RFX) gene that encodes transcription factors that contain five conserved domains. It binds to the X boxes of MHC class II genes and is essential for their expression. This transcription factor has been shown to regulate a wide variety of genes involved in immunity and cancer, including the MHC class II genes and genes that may be involved in cancer progression. The transcriptional factor encoded by RFX1 interacts with the product of the ABL1 gene, which is a proto-oncogene, and therefore may be associated with malignancies. This gene exhibits altered expression in glioblastoma and in the autoimmune disease systemic lupus erythematosis (SLE).

DHX36 is Probable ATP-Dependent RNA Helicase DHX36 on chromosome 3. It encodes a RNA helicase and acts to alter RNA secondary structure; it is involved in processes such as initiation of translation and nuclear or mitochondrial splicing. The protein encoded by DHX36 is also known as DEAH box protein 36, MLE-like protein 1, or G4 resolvase. This gene is believed to be involved in embryogenesis, spermatogenesis, and cellular growth and division with effects on mRNA stability. Mouse insertional mutagenesis experiments support DHX36 as a cancer causing gene in colorectal and central nervous system cancers.

MBD2 is Methyl-CpG-Binding Domain Protein 2 on chromosome 18. It encodes a protein that acts to regulate transcription and thus may affect differentiation and replication. The protein encoded by this gene may function as a mediator of the biological consequences of the methylation signal. It is also reported that this protein functions as a demethylase to activate transcription, as DNA methylation causes gene silencing. It is a transcription activator and is related to the Notch-1 and NF-κB pathways. Mouse insertional mutagenesis experiments support MBD2 as a cancer causing gene in blood, colorectal, central nervous system, and pancreatic cancers.

TRBV10-1 is T-Cell Receptor Beta Variable 10 on chromosome 7 and encodes a protein that is a variable domain of a T-cell receptor. As such, it is involved in cellular signaling and regulation.

MBD2, Methyl-CpG Binding Domain 2 on chromosome 18, is a protein coding gene that encodes a protein that is capable of binding to methylated DNA and can repress transcription from methylated gene promoters. Mouse insertional mutagenesis experiments support MBD2 as a cancer-causing gene in blood, colorectal, CNS, and pancreatic cancers.

FRK, Fyn Related Src Family Tyrosine Kinase, on chromosome 6, is a protein coding gene that encodes a tyrosine kinase that negatively regulates cell proliferation and positively regulates PTEN protein stability through phosphorylation of PTEN on Tyr³³⁶, which in turn prevents its ubiquitination and degradation; the protein is a nuclear protein and may function as a tumor suppressor. The protein may function during the G1 and S phases of the cell cycle and suppress growth. The protein has also been shown to interact with retinoblastoma protein and to be involved in cellular proliferation. Mouse insertional mutagenesis experiments support FRK as a cancer causing gene in pancreatic cancers.

RNF222, Ring Finger Protein 222, on chromosome 17, is a protein coding gene that has ubiquitin protein ligase activity.

PEG3, Paternally Expressed 3, on chromosome 19, is a protein coding gene that encodes a Kruppel-type zinc finger protein and may play a role in cell proliferation and p53-mediated apoptosis; it is a transcription factor that binds to DNA and affects proliferation. The protein induces apoptosis in cooperation with SIAH1A and acts as a mediator between p53/TP53 and BAX in a neuronal death pathway that is activated by DNA damage and acts synergistically with TRAF2 and inhibits TNF induced apoptosis through activation of NF-κB; PEG3 is an imprinted gene expressed exclusively from the paternal allele and plays important roles in controlling fetal growth rates with potential roles in mammalian reproduction. PEG3 may play a role in cell proliferation and p53-mediated apoptosis and has shown tumor suppressor activity and tumorigenesis activity in glioma and ovarian cells.

CYP11B2, Cytochrome P450, Family 11, Subfamily B, Member 2, on chromosome 8, is a protein coding gene that encodes a cytochrome P450 enzyme that is a monooxygenase that has steroid 18-hydroxylase activity to synthesize aldosterone and 18-oxocortisol as well as steroid 11 beta-hydroxylase activity.

NDC80, Kinetochore Complex Component, on chromosome 18, is a protein coding gene that encodes a protein consisting of an N-terminal microtubule binding domain and a C-terminal coiled-coiled domain that interacts with other components of the complex; this protein functions to organize and stabilize microtubule-kinetochore interactions and is required for proper chromosome segregation and spindle checkpoint activity; this surveillance mechanism assures correct segregation of chromosomes during cell division by detecting unaligned chromosomes and causing prometaphase arrest until the proper bipolar attachment of chromosomes is achieved, e.g., chromosome congregation during mitosis. This protein is also known as retinoblastoma-associated protein HEC. HEC is one of the proteins involved in spindle checkpoint signaling.

AP3B1, Adaptor Related Protein Complex 3 Beta 1 Subunit, on chromosome 5, is a protein coding gene encoding a protein that may play a role in organelle biogenesis associated with melanosomes, platelet dense granules, and lysosomes; the encoded protein is part of the heterotetrameric AP-3 protein complex which interacts with the scaffolding protein clathrin. Mutations in AP3B1 are associated with type 2 Hermansky-Pudlak syndrome. Mouse insertional mutagenesis experiments support AP3B1 as a cancer causing gene in colorectal, CNS, gastric, and liver cancers.

ZRSR1, on chromosome 5, is a protein coding gene encoding the U2 small nuclear ribonucleoprotein auxiliary factor 35-kDa subunit-related protein 1; this is a zinc finger DNA binding protein.

ABCA6, ATP Binding Cassette, Subfamily A, Member 6, on chromosome 17, is a protein coding gene encoding a member of the superfamily of ATP-binding cassette (ABC) transporters; this protein may play a role in macrophage lipid homeostasis and cellular proliferation. ABC proteins transport various molecules across extra- and intracellular membranes. These genes are divided into seven distinct subfamilies (ABC1, MDR/TAP, MRP, ALD, OABP, GCN20, and White). This specific encoded protein is a member of the ABC1 subfamily. Members of the ABC1 subfamily comprise the only major ABC subfamily found exclusively in multicellular eukaryotes. This gene is clustered among 4 other ABC1 family members on chromosome 17 at q24. Mouse insertional mutagenesis experiments support ABCA6 as a cancer-causing gene in liver cancers.

ANKLE1 is a protein coding gene that encodes an endonuclease that may play a role in DNA damage response and DNA repair.

CROCC, Ciliary Rootlet Coiled-Coil on chromosome 1 that encodes a protein that is a component of the ciliary rootlet and is also known as rootletin. Together with CEP68 and CEP250, it is required for centrosome cohesion and mutations can impact mitosis.

CYP39A1, Cytochrome P450 Family 39, Subfamily A, on chromosome 6, encodes a cytochrome P450 enzyme that is a monooxygenase and that is involved in the conversion of cholesterol to bile acids.

GSC2, Goosecoid Homeobox 2, on chromosome 22, is a protein coding gene that is a regulatory transcription factor involved in development. Mouse insertional mutagenesis experiments support GSC2 as a cancer causing gene in colorectal cancers. High levels of GSC2 expression in cancer cells correlates with poor survival rates.

LCMT1, Leucine Carboxyl Methyltransferase, on chromosome 16, is a protein coding gene that encodes an enzyme catalyzing the methylation of the carboxyl group of the C-terminal leucine residue (Leu³⁰⁹) of the catalytic subunit of protein phosphatase-2A.

METTL15, Methyltransferase Like 15, on chromosome 11, is a protein coding gene that most probably encodes a S-adenosyl-L-methionine-dependent methyltransferase.

PCNT, Pericentrin, on chromosome 21, is a protein coding gene that encodes a protein that binds to calmodulin and is likely important for normal functioning of the centrosomes, cytoskeleton, and cell-cycle progression.

PDCD6IP, Programmed Cell Death 6 Interacting Protein, on chromosome 3, is a protein coding gene that encodes a multifunctional protein involved in endocytosis, multivesicular body biogenesis, membrane repair, cytokinesis, apoptosis and maintenance of tight junction integrity. This gene encodes a protein that functions within the ESCRT pathway in the abscission stage of cytogenesis, in intralumenal endosomal vesicle expression, and in enveloped virus budding. Studies using mouse cells have shown that overexpression of this protein can block apoptosis. In addition, the product of this gene binds to the product of the PDCD6 gene, a protein required for apoptosis, in a calcium-dependent manner. Mouse insertional mutagenesis experiments support PDCD6IP as a cancer causing gene in colorectal, liver, and pancreatic cancers.

RBMXL1, RNA Binding Motif Protein, X-Linked Like 1, on chromosome 1, is a protein coding gene that may be involved in pre-mRNA splicing.

MSH3 (MutS Homolog 3), located on chromosome 5, is a protein coding gene. Diseases associated with MSH3 mutation include adenomatous polyposis and endometrial cancer. It is a familial component of the post-replicative DNA mismatch repair system (MMR) and is involved in genomic instability and is implicated in clonal evolution in gliomas.

TP53BP1, also known as “TP53,” is a gene located on chromosome 15, also known as Tumor Suppressor P53-Binding Protein 1, which encodes a protein that functions in the DNA double-strand break repair pathway choice (apoptosis pathway), promoting non-homologous end joining (NHEJ) pathways, and limiting homologous recombination. This protein plays multiple roles in the DNA damage response, including promoting checkpoint signaling following DNA damage, acting as a scaffold for recruitment of DNA damage response proteins to damaged chromatin, and promoting NHEJ pathways by limiting end resection following a double-strand break. TP53 has been linked to mutations present in post-temozolomide treated gliomas.

ADAM32, located on chromosome 8, also known as A Disintegrin And Metalloproteinase Domain 32, is a gene encoding a member of the disintegrin family of membrane anchored proteins. These genes play a role in diverse biological processes such as brain development, fertilization, tumor development, and inflammation. The protein expressed by ADAM32 is present in reproductive organs. Mutations in this gene have been associated with hypermutation in gliomas.

GPR116, also known as ADGRF5, is located on chromosome 6 and encodes a G protein-coupled receptor (GPR), G protein-coupled receptor 116. GPRs are cell surface receptors that activate guanine-nucleotide binding proteins upon the binding of a ligand. GPRs may play a role in neuron survival through activation of a downstream signaling pathway involving the PI3, Akt and MAP kinases. GPR116 has been identified as a hypermutating gene in gliomas after temozolomide treatment. GPRs are involved in cellular proliferation and evading apoptosis.

MUC16, located on chromosome 19, encodes a protein that is a member of the mucin family. This protein is thought to play a role in forming a barrier, protecting epithelial cells from pathogens. Products of this gene have been used as a marker for different cancers (e.g., ovarian carcinoma), with higher expression levels associated with poorer outcomes. MUC16 is involved in cell migration and is implicated in mutations present in post-temozolomide treated gliomas.

Given that all WHO Grade II and III gliomas will follow a path of mutation if they recur or progress, one logical approach to control of these gliomas would be to mitigate the rate and extent of mutations these tumors can express. While causal proof that eflornithine impaired mutation rates in patients with anaplastic gliomas treated (7,8) is lacking at present, as discussed previously, circumstantial evidence favors a role of eflornithine in mitigating tumor cell mutations. To recall the facts, eflornithine can produce G1-arrest in neuroblastoma, a neuroectodermal tumor like glioma, by increasing intracellular p21 and p27kip-1 proteins (9,10) and, without much doubt, impact tumor cell mutation rates. It was previously found (16) that topical eflornithine treatment of biopsied skin actinic keratosis reduced the percentage of p53-positive cells (22%; P=0.04) but not the frequency of p53 mutations compared to the placebo-treated skin.

This hypothesis can be evaluated by experiments that could prove or support the conclusion that eflornithine can reduce mutation rates in low- and mid-grade gliomas. However, these tumors generally grow poorly outside of the human host. As a result representative glial and non-glial tumors were used that grew in 3-dimensional culture and that do not show many mutations at the outset of their growth in culture. In another alternative, the experiments can be conducted in monolayer cultures.

Another approach would be to use Big Blue Rat-2 cells in a similar approach to that used to evaluate the mutagenic potential of TMZ (15). In addition to looking at DNA adducts they looked at lacI mutations in Big Blue Rat-2 cells and found a dose-dependent increase in lacI mutation frequency from 9.1 to 48.9 and 89.7 treated with TMZ at 0, 0.5, or 1 mM TMZ. Sequence analysis of the lacI mutants from the TMZ treatment group demonstrated that they were GC→AT transitions at non-CpG sites, which is significantly different from the mutation spectrum observed in the control treatment group. It is thus conceivable that one could treat Big Blue Rat-2 cells with eflornithine after a defined dose and duration of TMZ exposure has been given to initiate the mutation cascade.

Another possible approach would be to take a slowly developing IC rodent tumor that kills animals over a time period of about 6 months and look into the mutations that occur at 2, 4, and 6 months and divide mice into two groups. Group 1 consisted of eflornithine (1.5-2%) in drinking water for 3-weeks/4-weeks for months 2-6 versus Group 2 which did not receive eflornithine. This approach using ˜500 gene sequencing on each tumor tissue sample obtained at euthanasia at 2, 4, and 6 months after tumor implantation might be sufficient to provide the information and proof of eflornithine effectiveness on mutation frequency.

Techniques previously described (23,24) for growing glioma or adenocarcinoma cells in three-dimensional culture and then evaluating the effect of treatment with single agents or drug combinations can be used. These techniques were originally developed to rapidly isolate phosphoproteins from 3-dimensional cultures under conditions of serum starvation or hypoxia (25,26), but these techniques will work equally well for DNA and RNA isolates. In one instance, the monofunctional alkylating agent, temozolomide, was used to produce mutation and then to give eflornithine afterwards at 2 doses and 2 times to determine how well eflornithine reduces the mutation frequency of the tumor cells. Since temozolomide and eflornithine can be studied in such three-dimensional cultures with good results (23,24), it is expected that it would be possible to establish culture conditions for looking at mutation frequencies using 500-800 gene chip arrays at each time and dose point.

Derivatives and analogs of eflornithine include, but are not limited to, the following derivatives or analogs.

U.S. Pat. No. 5,614,557 to Bey et al. discloses analogs of eflornithine of Formula (III):

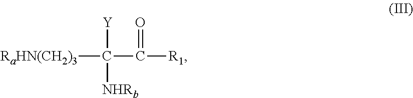

wherein:

(1) Y is FCH₂—, F₂CH—, or F₃C—;

(2) Rₐ and R_{b} are, independently, hydrogen, (C₁-C₄) alkylcarbonyl, or a group of Formula (III(a))

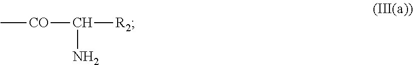

wherein, in Formula (III(a)), R₂ is hydrogen, (C₁-C₄) alkyl, benzyl, or p-hydroxybenzyl;

(3) R₁ is hydroxyl, (C₁-C₈) alkoxy, —NR₄R₅, wherein R₄ and R₅ are independently hydrogen, (C₁-C₄) alkyl, or a group of Formula (III(b))

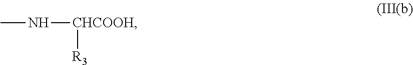

wherein, in Formula (III(b), R₃ is hydrogen, C₁-C₄) alkyl, or p-hydroxybenzyl.

U.S. Pat. No. 5,002,879 to Bowlin et al. discloses additional ornithine decarboxylase inhibitors of Formulas (IV) and (V):

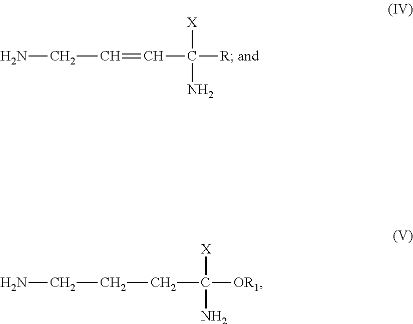

wherein:

(1) X is —CHF₂ or —CH₂F;

(2) R is hydrogen or —COR₁; and

(3) R₁ is —OH or (C₁-C₆) alkoxy.

Water-soluble salts of eflornithine with polycations such as polycationic carbohydrates (chitosan, water-soluble chitosan derivative, or a salt thereof) or a polyaminoacid, a polyamine, a polypeptide, a basic polymer, or a quaternary ammonium compound are disclosed in United States Patent Application Publication No. 2002/0019338 by Hebert. All pharmaceutically acceptable salt forms, hydrates, and solvates of eflornithine and derivatives, analogs, and prodrugs can be used in methods and compositions of the present invention.

Additional derivatives, analogs, and prodrugs of eflornithine are known in the art. United States Patent Application Publication No. 2010/0120727 by Xu discloses conjugates in which a first moiety that is eflornithine or a derivative or analog of eflornithine is covalently linked to a second moiety that is a non-steroidal anti-inflammatory drug (NSAID). The NSAID can be, for example, aspirin, aceclofenac, acemethacin, alclofenac, amoxiprin, ampyrone, azapropazone, benorylate, bromfenac, choline and magnesium salicylates, choline salicylate, celecoxib, clofezone, diclofenac potassium, diclofenac sodium, diclofenac sodium with misoprostol, diflunisal, droxicam, lornoxicam, meloxicam, tenoxicam, ethenzamide, etodolac, fenoprofen calcium, faislamine, flurbiprofen, flufenamic acid, ibuprofen, ibuproxam, indoprofen, alminoprofen, carprofen, dexibuprofen, dexketoprofen, fenbufen, flunoxaprofen, indomethacin, ketoprofen, ketorolac, kebuzone, loxoprofen, magnesium salicylate, meclofenamate sodium, metamizole, mofebutazone, oxyphenbutazone, phenazone, sulfinpyrazone, mefenamic acid, meloxicam, methyl salicylate, nabumetone, naproxen, naproxen sodium, nebumetone, oxaprozin, oxametacin, phenylbutazone, proglumetacin, piroxicam, pirprofen, suprofen, rofecoxib, salsalate, salicyl salicylate, salicylamide, sodium salicylate, sulindac, tiaprofenic acid, tolfenamic acid, tolmetin sodium, and valdecoxib. The first and second moieties can be linked via a covalent bond selected from the group consisting of an ester bond, an amide bond, an imine bond, a carbamate bond, a carbonate bond, a thioester bond, an acyloxycarbamate bond, an acyloxycarbonate bond, an acyloxythiocarbamate, a phosphate bond, a phosphoramidate and an acyloxyphosphate bond.

United States Patent Application Publication No. 2015/0306241 by Zhu et al. discloses copolymers of formula A-B-C or a pharmaceutically acceptable salt thereof, wherein A comprises a water soluble polymer; B comprises a matrix metalloprotease (MMP)-cleavable polypeptide; C is a chemotherapeutic drug or a derivative thereof; and A is connected to B at a first end through a first covalent bond or a first linking moiety and B is connected to C at a second end through a second covalent bond or a second linking moiety, and wherein the co-polymer is not crosslinked. Typically, in this copolymer, the chemotherapeutic drug is an amino-containing therapeutic drug, such as eflornithine.

United States Patent Application Publication No. 2002/0110590 by Shaked et al. discloses formulations for the administration of eflornithine, including a core having a rapid release DFMO-containing granules and a slow release granule and an outer layer surrounding the core comprising a pH responsive coating.

U.S. Pat. No. 9,034,319 to Teichberg et al. discloses the use of eflornithine together with an agent which reduces blood glutamate levels and enhances brain to blood glutamate efflux. The agent that reduces blood glutamate levels and enhances brain to blood glutamate efflux can be: (1) a transaminase that can be selected from the group consisting of glutamate oxaloacetate transaminase, glutamate pyruvate transaminase, acetylornithine transaminase, ornithine-oxo-acid transaminase, succinyldiaminopimelate transaminase, 4-aminobutyrate transaminase, (s)-3-amino-2-methylpropionate transaminase, 4-hydroxyglutamate transaminase, diiodotyrosine transaminase, thyroid-hormone transaminase, tryptophan transaminase, diamine transaminase, cysteine transaminase, L-Lysine 6-transaminase, histidine transaminase, 2-aminoadipate transaminase, glycine transaminase, branched-chain-amino-acid transaminase, 5-aminovalerate transaminase, dihydroxyphenylalanine transaminase, tyrosine transaminase, phosphoserine transaminase, taurine transaminase, aromatic-amino-acid transaminase, aromatic-amino-acid-glyoxylate transaminase, leucine transaminase, 2-aminohexanoate transaminase, ornithine(lysine) transaminase, kynurenine-oxoglutarate transaminase, D-4-hydroxyphenylglycine transaminase, cysteine-conjugate transaminase, 2,5-diaminovalerate transaminase, histidinol-phosphate transaminase, diaminobutyrate-2-oxoglutarate transaminase, and udp-2-acetamido-4-amino-2,4,6-trideoxyglucose transaminase; (2) a glutamate dehydrogenase; (3) a glutamate decarboxylase; (4) a glutamate-ethylamine ligase; (5) a transferase that can be selected from the group consisting of glutamate N-acetyltransferase and adenylyltransferase; (6) an aminomutase that can be glutamate-1-semialdehyde 2,1-aminomutase; and (7) a racemase. The enzyme can be used with a cofactor.

U.S. Pat. No. 6,277,411 to Shaked et al. discloses preparations comprising a capsule, tablet or other dosage form containing a core of different types of eflornithine.

U.S. Pat. No. 9,700,633 to Wang et al. discloses conjugates of water soluble polymer-amino acid oligopeptide-drug; the drug can be eflornithine. The conjugates have the structure shown in Formula (D-I):

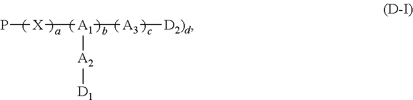

wherein:

(1) P is a water soluble polymer;

(2) X is a linking group, wherein the linking group links P and A₁;

(3) each of A₁ is independently same or different amino acid residue or amino acid analogue;

(4) each of A₂ and A₃ is independently alanine or valine;

(5) each of D₁ and D₂ is independently the same or a different drug molecule, one of which is eflornithine;

(6) a is 0 or 1;

(7) b is an integer of 2-12;

(8) c is an integer of 0-7; and

(9) d is 0 or 1.

U.S. Pat. No. 6,752,981 to Erion et al. discloses prodrugs for liver-specific delivery, including prodrugs that are prodrugs of eflornithine. The prodrugs are of Formula (D-II):

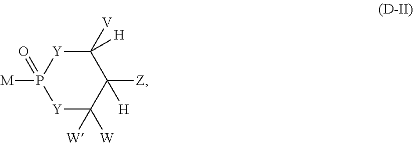

wherein:

(1) V, W, and W′ are independently selected from the group of —H, alkyl, aralkyl, alicyclic, aryl, substituted aryl, heteroaryl, substituted heteroaryl, 1-alkenyl, and 1-alkynyl; or together V and Z are connected via an additional 3-5 atoms to form a cyclic group containing 5-7 atoms, optionally 1 heteroatom, substituted with hydroxy, acyloxy, alkoxycarbonyloxy, or aryloxycarbonyloxy attached to a carbon atom that is three atoms from both Y groups attached to the phosphorus; or together V and Z are connected via an additional 3-5 atoms to form a cyclic group, optionally containing 1 heteroatom, said cyclic group is fused to an aryl group at the beta and gamma position to the Y adjacent to V;

(2) together V and W are connected via an additional 3 carbon atoms to form an optionally substituted cyclic group containing 6 carbon atoms and substituted with one substituent selected from the group of hydroxy, acyloxy, alkoxycarbonyloxy, alkylthiocarbonyloxy, and aryloxycarbonyloxy, attached to one of said additional carbon atoms that is three atoms from a Y attached to the phosphorus; together Z and W are connected via an additional 3-5 atoms to form a cyclic group, optionally containing one heteroatom, and V must be aryl, substituted aryl, heteroaryl, or substituted heteroaryl; (3) together W and W′ are connected via an additional 2-5 atoms to form a cyclic group, optionally containing 0-2 heteroatoms, and V must be aryl, substituted aryl, heteroaryl, or substituted heteroaryl;

(4) Z is selected from the group of —CHR²OH, —CHR²OC(O)R³, —CHR²OC(S)R³, —CHR²OC(S)OR³, —CHR²OC(O)SR³, —CHR²OCO₂R³, —OR², —SR², —CHR²N₃, —CH₂aryl, —CH(aryl)OH, —CH(CH═CR²₂)OH, —CH(C≡R²)OH, —R², —NR²₂, —OCOR³, —OCO₂R³, —SCOR³, —SCO₂R³, —NHCOR², —NHCO₂R³, —CH₂NHaryl, —(CH₂)ₚ—OR¹², and —(CH₂)ₚ—SR¹²;

(5) p is an integer 2 or 3;

(6) with the provisos that: (i) V, Z, W, W′ are not all —H; (ii) when Z is —R² or —OR², then V is not —H, alkyl, aralkyl, or alicyclic; (iii) when Z is CHR²OH, then M is not —NH(lower alkyl), —N(lower alkyl)₂, —NH(lower alkyl halide), —N(lower alkyl halide)₂ or —N(lower alkyl)(lower alkyl halide); and (iv) when V is aryl or substituted aryl, then M is not —O(D) where D is hydrogen, a metal ion or an ammonium ion;

(7) R² is selected from the group of R³ and —H;

(8) R³ is selected from the group of alkyl, aryl, alicyclic, and aralkyl;

(9) R⁶ is selected from the group of —H, lower alkyl, acyloxyalkyl, alkoxycarbonyloxyalkyl, and lower acyl;

(10) R¹² is selected from the group of —H and lower acyl;

(11) each Y is independently selected from the group of —O— and —NR⁶—; and

(12) M is selected from the group of drugs MH containing an —OH, —NHR², or —SH group, and that is attached to the phosphorus in formula I via O, N, or S of the OH, —NHR², or SH group; wherein the drug MH can be eflornithine.

U.S. Pat. No. 6,602,915 to Uhrich discloses polyazo derivatives of eflornithine that comprise a polymer comprising a backbone, wherein the backbone comprises one or more azo linkages, and wherein the backbone comprises one or more groups that will yield eflornithine upon hydrolysis of the polymer.

U.S. Pat. No. 8,299,291 to Raillard et al. discloses 1-(acyloxy)-alkyl carbamate prodrugs, including prodrugs of eflornithine.

U.S. Pat. No. 8,241,668 to Uhrich discloses polyesters and polyamides that can incorporate eflornithine and function as prodrugs; the polyesters or polyamides are of Formula (D-III):

—R₂-A-L-A-R₃-A-L-A- (D-III)

wherein:

(1) R₂ and R₃ are each independently a group that will yield an anticancer agent upon hydrolysis of the polymer; the anticancer agent is eflornithine;

(2) each A is independently an amide or ester linkage; and

(3)) each L is independently a linking group.

U.S. Pat. No. 7,303,739 to Erion et al. discloses prodrugs for liver-specific delivery, including conjugates incorporating eflornithine; the prodrugs are of Formula (D-IV):

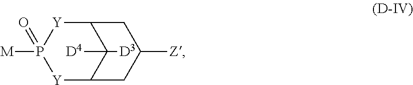

wherein:

(1) Z′ is selected from the group consisting of —OH, —OC(O)R³, —OCO₂R³, and —OC(S)R³;

(2) D³ and D⁴ are independently selected from the group consisting of —H, alkyl, —OH, and —OC(O)R³;

(3) R² is independently selected from the group consisting of R³ and —H;

(4) R³ is selected from the group consisting of alkyl, aryl, alicyclic, and aralkyl;

(5) each Y is selected from the group consisting of —O— and —NR⁶—; and

(6) M is eflornithine and is attached to the P of Formula (D-IV) via O, N, or S or the —OH, —NHR², or —SH group.

U.S. Pat. No. 7,186,753 to Del Soldato discloses nitro derivatives of eflornithine, including derivatives of Formula (D-V):

A-B—N(O)ₛ (D-V),

wherein:

(1) s is 1 or 2;

(2) A is R-T₁, wherein: (i) R is a drug radical wherein the drug is eflornithine; and T₁ is (CO)ₜ or (X)ᵣ, wherein X is O, S, or NR_{1C}, R_{1C} is hydrogen or a linear or branched alkyl having from 1 to 6 carbon atoms or a free valence, t and t′ are integers and equal to 0 or 1, with the proviso that t is 1 when t′ is 0 and t is 0 when t′ is 1;

(3) B is -T_{B}-X₂—O— wherein: X₂ is a R_{1B}—X—R_{2B} moiety wherein X is as defined above in (2), R_{1B} and R_{2B} are the same or different and are linear or branched C₁-C₆ alkylene moieties, or X₂ is a radical wherein two alkylene two alkylene chains C₁-C₄ are linked to nonadjacent positions of a central ring having 4 or 6 atoms, said ring being an unsaturated cycloaliphatic ring, or a saturated or aromatic heterocylic ring, containing one or two heteroatoms, the same or different, selected from O, S, and N; and

(4) wherein the unsaturated cycloaliphatic ring does not have aromatic character according to Hückel's rule.

U.S. Pat. No. 6,630,511 to Hebert discloses a salt of eflornithine and chitosan.

U.S. Pat. No. 7,345,196 to Majeed et al. discloses additional analogs and derivatives of eflornithine of Formula (D-V):

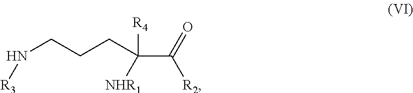

wherein:

(1) R₁ is hydrogen;

(2) R₂ is hydroxy, C₁-C₈ alkoxy, or C₁-C₈—NR′R″ wherein R′ and R″ are independently hydrogen or C₁-C₈ alkyl;

(3) R₃ is hydrogen; and

(4) R₄ is —CH₂F, —CHF₂, or —CF₃.

United States Patent Application Publication No. 2018/0125986 by Satyam discloses nitric oxide releasing prodrugs of eflornithine.

United States Patent Application Publication No. 2018/0104349 by Qin et al. discloses conjugates of eflornithine with bifunctional linkers, wherein the eflornithine is coupled to a targeting moiety; one end of the linker is linked to the eflornithine and the other end of the linker specifically and covalently links a targeting substance site under the action of sortase enzyme.

United States Patent Application Publication No. 2017/0209595 by Zhao discloses conjugates incorporating hydrazine, disulfur bridge linkers, and eflornithine.

United States Patent Application Publication No. 2017/0173168 by Zhao discloses conjugates incorporating acetylenedicarboxyl linkers and eflornithine, wherein the acetylenedicarboxyl group in the linker is capable of reacting with a pair of sulfur atoms of a cell-binding agent and wherein the conjugate comprises either two molecules of eflornithine or one molecule of eflornithine and one molecule of another drug.

United States Patent Application Publication No. 2017/0152274 by Zhao et al. discloses conjugates incorporating phosphinate-based charged linkers and eflornithine for conjugation of eflornithine to an antibody or other cell-binding agent.

United States Patent Application Publication No. 2017/0151346 by Zhao discloses conjugates incorporating disulfur bridge linkers and eflornithine, wherein the disulfur bridge linkers contain hydrazine for conjugation of eflornithine to an antibody or other cell-binding agent through a bridge linking a pair of thiols on the antibody or other cell-binding agent.

United States Patent Application Publication No. 2017/0143845 by Zhao discloses acetylenedicarboxyl linkers for conjugation of eflornithine to an antibody or other cell-binding agent through two disulfide groups wherein the linker binds two eflornithine molecules.

United States Patent Application Publication No. 2016/0076021 by Stojanovic et al. discloses aptamers in which the target molecule can include an amino acid such as eflornithine.

United States Patent Application Publication No. 2015/0306241 by Zhu et al. discloses conjugates including copolymers for delivery of eflornithine. The copolymers are of the formula A-B-C, wherein: (i) A is a water-soluble polymer; (ii) B is a matrix metalloprotease (MMP)-cleavable polypeptide; and (iii) C is eflornithine or a derivative thereof, wherein A is connected to B at a first end through a first covalent bond or a first linking moiety and B is connected to C at a second end through a second covalent bond or a second linking moiety, and wherein the copolymer is not cross-linked.

United States Patent Application Publication No. 2015/0284416 by Zhao discloses conjugates incorporating hydrophilic linkers and eflornithine. The linkers can be bound to a cell-binding agent such as an antibody.

United States Patent Application Publication No. 2015/0158809 by Wang et al. discloses 1-acyloxy-alkylcarbamate prodrugs of eflornithine of Formula (D-VI):

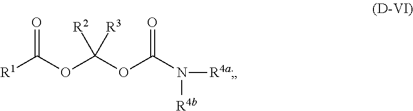

wherein:

(1) each of R¹ and R² is independently C₁-C₄ alkyl;

(2) R³ is H or C₁-C₄ alkyl;

(3) HNR^{4a}R₄^{b} is a drug molecule having an amino moiety that can be eflornithine;

(4) R^{4a} and R^{4b} are groups of the drug molecule (eflornithine) attached to the amino moiety;

(5) each of R^{5a}, R^{5b}, R^{5c}, R^{5d}, and R^{5e} is independently selected from H, halo, C₁-C₄ alkyl, halo-C₁-C₄ alkyl, phenyl, —C(O)O—C₁-C₄ alkyl, —C(O)—C₁-C₄ alkyl, —S(O)—C₁-C₄ alkyl, —CN, —C(O)—NR^{6a}R^{6b}, substituted or unsubstituted C₁-C₄ alkoxy, and substituted or unsubstituted phenoxy;

(6) each of R^{6a} and R^{6b} is independently H or C₁-C₄ alkyl, or R^{6a} and R^{6b} together with the nitrogen atom to which they are attached form a heterocycle;

(7) provided that at least one of R^{5a}, R^{5b}, R^{5c}, R^{5d}, and R^{5e} is other than H; or any two adjacent R^{5a}, R^{5b}, R^{5c}, R^{5d}, and R^{5e} are joined together to form a carbocyclic or heterocyclic moiety; and

(8) X is a leaving group.

United States Patent Application Publication No. 2015/0057221 by Cleemann et al. discloses eflornithine prodrugs comprising a drug linker conjugate, the prodrugs are linked to a non-biologically-active linker that includes an amine-containing nucleophile wherein the eflornithine is attached via a nitrogen atom by formation of an amide bond.

United States Patent Application Publication No. 2011/0263526 by Satyam discloses nitric oxide releasing prodrugs of eflornithine.

United States Patent Application Publication No. 2010/0120727 by Xu discloses a covalent conjugate of an eflornithine analog with a non-steroidal anti-inflammatory drug (NSAID). The NSAID can be selected from the group consisting of aspirin, aceclofenac, acemethacin, alclofenac, amoxiprin, ampyrone, azapropazone, benorylate, bromfenac, choline and magnesium salicylates, choline salicylate, celecoxib, clofezone, diclofenac potassium, diclofenac sodium, diclofenac sodium with misoprostol, diflunisal, droxicam, lornoxicam, meloxicam, tenoxicam, ethenzamide, etodolac, fenoprofen calcium, faislamine, flurbiprofen, flufenamic acid, ibuprofen, ibuproxam, indoprofen, alminoprofen, carprofen, dexibuprofen, dexketoprofen, fenbufen, flunoxaprofen, indomethacin, ketoprofen, ketorolac, kebuzone, loxoprofen, magnesium salicylate, meclofenamate sodium, metamizole, mofebutazone, oxyphenbutazone, phenazone, sulfinpyrazone, mefenamic acid, meloxicam, methyl salicylate, nabumetone, naproxen, naproxen sodium, nebumetone, oxaprozin, oxametacin, phenylbutazone, proglumetacin, piroxicam, pirprofen, suprofen, rofecoxib, salsalate, salicyl salicylate, salicylamide, sodium salicylate, sulindac, tiaprofenic acid, tolfenamic acid, tolmetin sodium, and valdecoxib. The covalent bond linking the eflornithine or derivative or analog and the non-steroidal anti-inflammatory drug can be selected from the group consisting of an ester bond, an amide bond, an imine bond, a carbamate bond, a carbonate bond, a thioester bond, an acyloxycarbamate bond, an acyloxycarbonate bond, an acyloxythiocarbamate, a phosphate bond, a phosphoramidate and an acyloxyphosphate bond. The conjugate can further comprise a linker.

United States Patent Application Publication No. 2009/0286732 by Hill et al. discloses conjugates of the form B-L-M for delivery of therapeutic agents to nerve cells, wherein B is a binding agent capable of binding to a nerve cell surface receptor and mediating absorption of the compound by the nerve cell, L is a linker, and M is a therapeutic agent such as eflornithine; the binding agent can be a neurotrophin or a fragment or derivative thereof.

United States Patent Application Publication No. 2009/0131342 by Ellis discloses nitrosated or nitrosylated derivatives of eflornithine.

United States Patent Application Publication No. 2008/0233078 by Uhrich discloses a polymer comprising a backbone, wherein the backbone comprises ester, thioester, or amide linkages, and wherein the backbone comprises one or more groups that will yield an anticancer agent upon hydrolysis of the polymer; the group that will yield an anticancer agent upon hydrolysis can be eflornithine.

United States Patent Application Publication No. 2007/0072800 by Gengrinovitch et al. discloses conjugates of eflornithine with an amino acid or an amino acid analog. The conjugates are of Formula (D-VII):

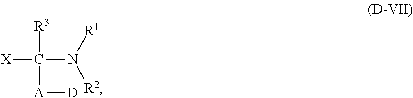

wherein:

(1) A denotes the side chain of an amino acid, the side chain having a functional group selected from the group consisting of an amino group, a carboxyl, a sulfhydryl and a hydroxyl;

(2) D is a residue of eflornithine;

(3) R¹ and R² are independently selected from a group consisting of hydrogen, a lower alkyl, an amino acid, a peptide of about 2 to about 50 amino acids, a C₁-C₂₀ fatty acid, a sugar moiety, a polymer chain and a group of Subformula (D-VII(a)):

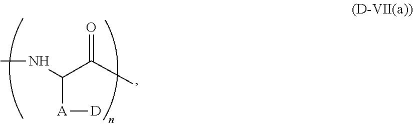

wherein, in Subformula (D-VII(a)), n is an integer of 1-20;

(4) R³ is selected from the group consisting of H and lower alkyl; and

(5) X is selected from the group consisting of a hydroxyl, an amide, a hydrazide, an ester, a thioester, an aldehyde, an amino acid and a peptide.

United States Patent Application Publication No. 2005/0053577 by Uhrich discloses conjugates of eflornithine with polyanhydrides. The polymer comprises a backbone, wherein the backbone comprises an anhydride linkage, and wherein the backbone comprises one or more groups that will yield a biologically active compound upon hydrolysis of the polymer (in this context, eflornithine); provided that the biologically active compound is not an ortho-hydroxy aryl carboxylic acid. Typically, the polymer comprises one or more units of Formula (D-VIII):

—C(═)R¹—X—R²—X—R¹—C(═O)—O— (D-VIII),

wherein:

(1) each R¹ is a group that will provide eflornithine upon hydrolysis of the polymer;

(2) each X is independently an amide linkage, a thioester linkage, or an ester linkage; and

(3) R² is a linking group.

United States Patent Application Publication No. 2004/0228832 by Uhrich discloses conjugates of eflornithine with polyazo compounds.

United States Patent Application Publication No. 2003/0105066 by Del Soldato discloses nitrate salts of eflornithine.

United States Patent Application Publication No. 2018/0021451 by Kim et al. discloses conjugates of an antibody and eflornithine having an amino acid motif that can be recognized by an isoprenoid transferase; the conjugate comprises:

(1) a full-length antibody comprising two immunoglobulin heavy chains and two immunoglobulin light chains, wherein the antibody recognizes and specifically binds to a target through at least one antigen recognition site;

(2) at least one amino acid motif having an amino acid sequence CAAX, wherein C represents cysteine, A represents an aliphatic amino acid, and X represents an amino acid that determines a substrate specificity of the isoprenoid transferase, directly or indirectly linked to a carboxyl terminus of a heavy chain or light chain of the antibody, wherein the amino acid motif is recognizable by an isoprenoid transferase;

(3) an isosubstrate directly linked to a cysteine moiety of the at least one amino acid motif, wherein the isosubstrate contains at least one isoprenoid unit and is recognizable by the isoprenoid transferase; and

(4) eflornithine, wherein the eflornithine is directly or indirectly linked to the isosubstrate.

United States Patent Application Publication No. 2016/0213786 by Kim et al. discloses protein-eflornithine conjugates including an antibody.

United States Patent Application Publication No. 2018/0118661 by Tavares et al. discloses analogs and derivatives of eflornithine of Formula (VII):

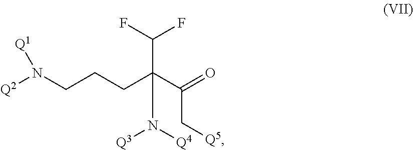

wherein one or more of Q¹, Q², Q³, Q⁴, and Q⁵ is a moiety which is cleavable in vivo to hydrogen and the remainder are hydrogen. The compounds include: (i) an N-acyloxyalkoxycarbonyl derivative of eflornithine; (ii) a phosphoryloxymethylcarbamate derivative of eflornithine; (iii) a redox based system derivative of eflornithine; (iv) a β-aminoketone derivative of eflornithine; (v) a Schiff base derivative of eflornithine; (vi) an N-Mannich base derivative of eflornithine; (vii) an (oxodioxolenyl)methylcarbamate of eflornithine; (viii) a trimethyl lock system based derivative of eflornithine; (ix) an intramolecular bonded derivative of eflornithine; (x) a tetrahydrothiadiazine-2-thione derivative of eflornithine; and (xi) a sulfonamide derivative of eflornithine.

United States Patent Application Publication No. 2017/0088621 by Kim et al. discloses conjugates of an anti-EGFR antibody and eflornithine comprising compounds of Formula (D-IX):

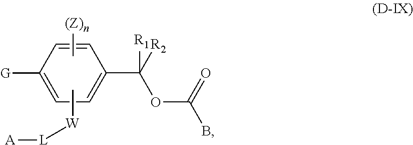

wherein:

(1) G is a residue of a sugar or sugar acid, preferably a residue of glucuronic acid or a derivative thereof;

(2) A represents the anti-EGFR antibody;

(3) B represents eflornithine;

(4) W represents an electron-withdrawing group, preferably —C(O)NR′—, where C(O) is bonded to the phenyl ring and NR′ is bonded to L;

(5) each Z independently represents hydrogen, C₁-C₈ alkyl, halogen, cyano, or nitro, preferably hydrogen;

(6) n is an integer of 0 to 3;

(7) L comprises a chain of 3 to 100 atoms that covalently links A to W; and

(8) R₁ and R₂ are each independently hydrogen, C₁-C₈ alkyl, or C₃-C₈ cycloalkyl, preferably hydrogen, or R₁ and R₂ taken together with the carbon atom to which they are attached form a C₃-C₈ cycloalkyl ring.

United States Patent Application Publication No. 2006/0235211 by Heindel et al. discloses conjugates of lactones of polysaccharide carboxylic acids and eflornithine.

Accordingly, as detailed further below, one aspect of the present invention is a method for the treatment of glioma comprising the step of administering a therapeutically effective quantity of eflornithine or a derivative or analog thereof to a subject with glioma in order to reduce the rate of hypermutation of the glioma to reduce the progression or grade of malignancy of the glioma caused by alkylating therapy exposure. Typically, the glioma is a WHO Grade II or Grade III glioma. In one alternative, the glioma is selected from the group consisting of anaplastic glioma, anaplastic oligodendroglioma, and anaplastic oligoastrocytoma (also known as mixed glioma).

As detailed below, eflornithine or a derivative or analog thereof can also be used to treat other malignancies, including, but not limited to, a non-glioma central nervous system malignancy, colorectal cancer, leukemia, lymphoma, pancreatic cancer, liver cancer, and stomach cancer. Still other malignancies can be treated by administration of eflornithine or a derivative or analog thereof.

Eflornithine or a derivative or analog thereof as described above can be used together with other agents. Pharmaceutically acceptable salt forms, hydrates, and solvates of eflornithine and derivatives, analogs, and prodrugs thereof can be used individually or together with other agents.

Some examples of the use of eflornithine or derivatives or analogs thereof with other agents are described below.

U.S. Pat. No. 9,150,495 to Phanstiel, IV discloses the use of eflornithine together with a polyamine transporter selective compound, including aromatic hydrocarbons di-substituted with a polyamine.

U.S. Pat. No. 9,072,778 to Bachmann discloses the use of eflornithine together with SAM486A (an S-adenosylmethionine decarboxylase inhibitor, 4-(aminoiminomethyl)-2,3-dihydro-1H-inden-1-one-diaminomethylenehydrazone), a retinoid, and an antineoplastic drug.

U.S. Pat. No. 8,597,904 to Bachmann et al. discloses use of eflornithine together with glidobactin, syringolin, and other syrbactin compounds.

U.S. Pat. No. 7,718,764 to Wong et al. discloses conjugates of eflornithine with peptides, including VAPEEHPTLLTEAPLNPK (SEQ ID NO: 1) and fragments and derivatives thereof, for use as an anti-neoplastic agent.

U.S. Pat. No. 7,655,678 to Gupta et al. discloses the use of eflornithine together with celecoxib. United States Patent Application Publication No. 2003/0203956 by Masterrer discloses the use of eflornithine with a cyclooxygenase-2 inhibitor selected from the group consisting of lumiracoxib, celecoxib, rofecoxib, etoricoxib, valdecoxib, parecoxib, and deracoxib. Similarly, U.S. Pat. No. 6,258,845 to Gerner et al. discloses the use of eflornithine together with the non-steroidal anti-inflammatory sulindac.

U.S. Pat. No. 7,432,302 to Burns et al. discloses the use of polyamine transport inhibitors together with eflornithine. The polyamine transport inhibitors can be compounds of structure R-X-L-polyamine wherein R is a straight or branched C₁₀-C₅₀ saturated or unsaturated aliphatic, carboxyalkyl, carbalkoxyalkyl, or alkoxy; a C₁-C₈ alicyclic moiety; a single or multiring aryl substituted or unsubstituted aliphatic; and aliphatic-substituted or unsubstituted single or multiring aromatic; a single or multiring heterocyclic; a single or multiring heterocyclic aliphatic; an aryl sulfonyl; X is —CO—, —SO₂—, or —CH₂—; and L is a covalent bond or a naturally occurring amino acid, lysine, ornithine, or 2,4-diaminobutyric acid.

U.S. Pat. No. 7,425,579 to Poulin et al. discloses the use of polyamine transport inhibitors together with eflornithine. The polyamine transport inhibitors can be compounds of Formula (PT-I) or (PT-II):

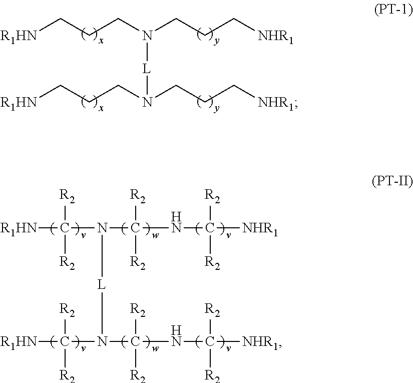

wherein: L is a linker; R₁ is hydrogen, methyl, ethyl, or propyl; R₂ is hydrogen or methyl; 0<x<3; 0<y<3; 2<v<5; and 2<w<8.

U.S. Pat. No. 7,208,528 to Vermeulin et al. discloses the use of polyamine transport inhibitors together with eflornithine. The polyamine transport inhibitors can be an N-monosubstituted polyamine analog or derivative of Formula (PT-III)

R—CO—NH—(CH₂)₃—NH—(CH₂)₄—NH—(CH₂)₃—NH₂ (PT-III),

wherein: R is selected from a D or L amino acid; D or L ornithine, an alicyclic, a single or multi-ring aromatic; aliphatic-substituted single or multi-ring aromatic; and a substituted or unsubstituted, single or multi-ring heterocyclic and wherein when R is a substituted single or multi-ring heterocyclic, heterocyclic is substituted with at least one member of the group consisting of: OH, halogen, NO₂, NH₂, NH(CH₂)ₙCH₃, N((CH₂)ₙCH₃)₂, CN, (CH₂)ₙCH₃, O(CH₂)ₙCH₃, S(CH₂)ₙCH₃, NHCO(CH₂)ₙCH₃, or O(CF₂)ₙCF₃, COO(CH₂)ₙCH₃, wherein n is 0-10.

U.S. Pat. No. 7,160,923 to Vermeulin et al. discloses the use of polyamine transport inhibitors together with eflornithine. The polyamine transport inhibitors can have the formula R₁—X—R₂, wherein R₁—X— is of the formula R—NH—CR′R″—CO—; wherein NH—CR′R″—CO— is a D- or L-form of valine, asparagine, or glutamine, or the D-form of lysine or arginine; wherein R″ is H, CH₃, CH₂CH₃, or CHF₂; where R is H or a head group selected from the group consisting of a straight or branched C₁-C₁₀ aliphatic, alicyclic, single or multiring aromatic, single or multiring aryl substituted aliphatic, aliphatic-substituted single or multiring aromatic, a single or multiring heterocyclic, a single or multiring heterocyclic-substituted aliphatic and an aliphatic-substituted aromatic; and wherein R₂ is a polyamine.

U.S. Pat. No. 7,144,920 to Burns et al. discloses polyamine analogs that induce antizyme activity and inhibit polyamine transporter activity and that can be used with eflornithine, including compounds of Formula (PT-IV):

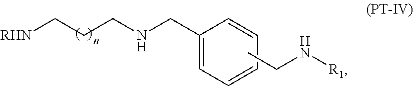

wherein: n can be 0 to 8 and the aminomethyl functionality can be ortho, meta or para substituted, R is hydrogen, 2-aminoethyl, 3-aminopropyl, 4-aminobutyl, 5-aminopentyl, 6-aminohexyl, 7-aminoheptyl, or 8-aminooctyl and R₁ is hydrogen and wherein the polyamine is non-symmetrical.

U.S. Pat. No. 7,094,808 to Bergeron, Jr. discloses polyamine transport inhibitors of Formula (PT-V):

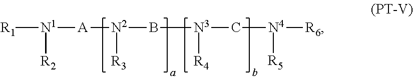

wherein: R₁-R₆ may be the same or different and are alkyl, aryl, aryl alkyl, or cycloalkyl, optionally having an alkyl chains interrupted by at least one etheric oxygen atom, or hydrogen; N¹, N², N³ and N⁴ are nitrogen atoms capable of protonation at physiological pH's; a and b may be the same or different and are integers from 1 to 4; A, B and C may be the same or different and are bridging groups which effectively maintain the distance between the nitrogen atoms such that the polyamine: (i) is capable of uptake by a target cell upon administration of the polyamine to a human or non-human animal; and (ii) upon uptake by the target cell, competitively binds via an electrostatic interaction between the positively charged nitrogen atoms to substantially the same biological counter-anions as the intra-cellular natural polyamines in the target cell; the polyamine, upon binding to the biological counter-anion in the cell, functions in a manner biologically different than the intracellular polyamines, the polyamine not occurring in nature.

U.S. Pat. No. 7,030,126 to Ramesh et al. discloses the use of the polyamine analog N(1),N(11)-diethylnorspermine (DENSPM), which can be used with eflornithine, as a polyamine synthesis inhibitor.

U.S. Pat. No. 6,963,010 to Burns et al. discloses the use of hydrophobic polyamine analogs that can be used with eflornithine. These analogs include analogs of Formulas (PT-VI), (PT-VII), (PT-VIII), and (PT-IX):

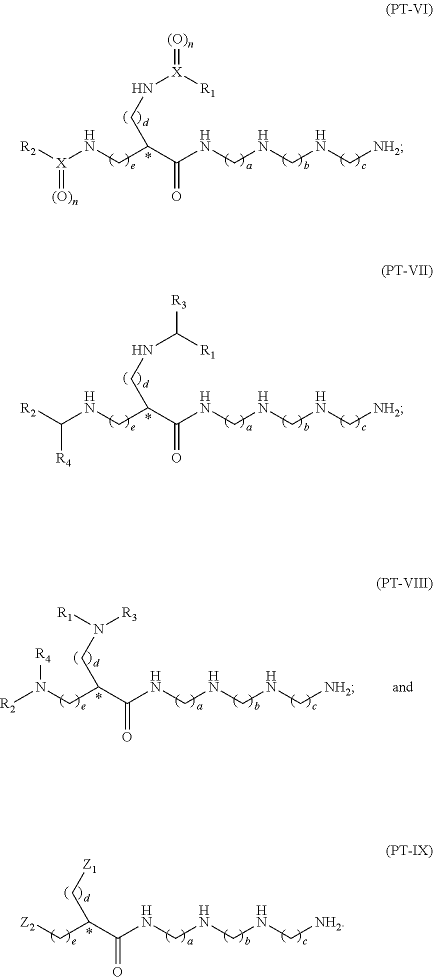

In compounds of Formula (PT-VI): a, b, and c independently range from 1 to 10; d and e independently range from 0 to 30; each X is independently either a carbon (C) or sulfur (S) atom, and R₁ and R₂ are independently selected from H or from the group of a straight or branched C₁-C₅₀ saturated or unsaturated aliphatic, carboxyalkyl, carbalkoxyalkyl, or alkoxy; a C₁-C₈ alicyclic; a single or multiring aryl substituted or unsubstituted aliphatic; an aliphatic-substituted or unsubstituted single or multiring aromatic; a single or multiring heterocyclic; a single or multiring heterocyclic aliphatic; a C₁-C₁₀ alkyl; an aryl sulfonyl; or cyano; or each of R₁X{O}ₙ— and R₂X{O}ₙ— are independently replaced by H; wherein * denotes a chiral carbon position; and wherein if X is C, then n is 1; if X is S, then n is 2; and if X is C, then the XO group may be CH₂ such that n is 0.

In compounds of Formula (PT-VII): a, b, and c independently range from 1 to 10 and d and e independently range from 0 to 30; and R₁, R₂, R₃, and R₄ may be the same or different and are independently selected from H or from the group of a straight or branched C₁-C₅₀ saturated or unsaturated aliphatic, carboxyalkyl, carbalkoxyalkyl, or alkoxy; a C₁-C₈ alicyclic; a single or multiring aryl substituted or unsubstituted aliphatic; an aliphatic-substituted or unsubstituted single or multiring aromatic; a single or multiring heterocyclic; a single or multiring heterocyclic aliphatic; a C₁-C₁₀ alkyl; an aryl sulfonyl; or cyano.

In compounds of Formula (PT-VIII): a, b, and c independently range from 1 to 10 and d and e independently range from 0 to 30; and R₁, R₂, R₃, and R₄ may be the same or different and are independently selected from H or from the group of a straight or branched C₁-C₅₀ saturated or unsaturated aliphatic, carboxyalkyl, carbalkoxyalkyl, or alkoxy; a C₁-C₈ alicyclic; a single or multiring aryl substituted or unsubstituted aliphatic; an aliphatic-substituted or unsubstituted single or multiring aromatic; a single or multiring heterocyclic; a single or multiring heterocyclic aliphatic; a C₁-C₁₀ alkyl; an aryl sulfonyl; or cyano.

In compounds of Formula (PT-IX): a, b, and c independently range from 1 to 10 and d and C independently range from 0 to 30; and wherein Z₁ is NR₁R₃ and Z₂ is selected from —R₁, —CHR₁R₂ or —CR₁R₂R₃ or Z₂ is NR₂R₄ and Z₁ is selected from —R₁, —CHR₁R₂ or —CR₁R₂R₃, wherein R₁, R₂, and R₃ may be the same or different and are independently selected from H or from the group of a straight or branched C₁-C₅₀ saturated or unsaturated aliphatic, carboxyalkyl, carbalkoxyalkyl, or alkoxy; a C₁-C₈ alicyclic; a single or multiring aryl substituted or unsubstituted aliphatic; an aliphatic-substituted or unsubstituted single or multiring aromatic; a single or multiring heterocyclic; a single or multiring heterocyclic aliphatic; a C₁-C₁₀ alkyl; an aryl sulfonyl; or cyano.

U.S. Pat. No. 6,872,852 to Burns et al. discloses polyamine analogs that can be used with eflornithine, including compounds of the formula R₁—X—R₂, wherein R₁ and R₂ are independently H or a moiety selected from the group consisting of a straight or branched C₁-C₁₀ aliphatic, alicyclic, single or multiring aromatic, single or multi-ring aryl substituted aliphatic, aliphatic-substituted single or multiring aromatic, a single or multiring heterocyclic, a single or multi-ring heterocyclic-substituted aliphatic and an aliphatic-substituted aromatic, and halogenated forms thereof; and X is a polyamine with two terminal amino groups, —(CH₂)₃—NH—, or —CH₂-Ph-CH₂—.

U.S. Pat. No. 6,646,149 to Vermeulen et al. discloses polyamine analogs that inhibit polyamine transporter activity and can be used with eflornithine, including compounds of the formula R₁—X—R₂, wherein R₁ and R₂ are each a polyamine or an analog or derivative of a polyamine and X is a linker moiety connecting the two polyamine moieties.

U.S. Pat. No. 6,392,098 to Frydman et al. discloses conformationally restricted polyamine analogs that can be used with eflornithine, including compounds of formula E-NH-D-NH—B-A-B—NH-D-NH-E, wherein: A is selected from the group consisting of C₂-C₆ alkenyl and C₃-C₆ cycloalkyl, cycloalkenyl, and cycloaryl; B is independently selected from the group consisting of a single bond and C₁-C₆ alkyl and alkenyl; D is independently selected from the group consisting of C₁-C₆ alkyl and alkenyl, and C₃-C₆ cycloalkyl, cycloalkenyl, and cycloaryl; E is independently selected from the group consisting of H, C₁-C₆ alkyl and alkenyl.

U.S. Pat. No. 6,083,496 to Poulin et al. discloses inhibitors of polyamine transport that can be used with eflornithine including synthetic derivatives of a dimer of an original polyamine, wherein the original polyamine is modified to comprise an amido group immediately linked to a carbon atom of the original polyamine and being located between two internal atoms, the dimer being linked together by a spacer side chain anchored to the amido group of each monomer.

U.S. Pat. No. 5,880,161 to Basu et al. discloses polyamine analogs that can be used with eflornithine, including molecules having a formula R₁—NH—(CH₂)_{w}—NH—(CH₂)ₓ—NH—(CH₂)_{y}—NH—(CH₂)_{z}—NH—R₂, wherein R₁ and R₂ are hydrocarbon chains of 1 to 5 carbons and w, x, y, and z are integers of 1 to 10; one preferred molecule is N¹, N¹⁹-bis-(ethylamino)-5,10,15-triazanonadecane.

U.S. Pat. No. 5,374,658 to Lau discloses use of oxidized polyamines including N,N′-bis-(3-propionaldehyde)-1,4-diaminobutane (spermine bisaldehyde) together with eflornithine.

U.S. Pat. No. 4,952,585 to Sunkara et al. discloses the use of eflornithine with esters of castanospermine. Similarly, U.S. Pat. No. 4,792,558 to Sunkara et al. discloses the use of eflornithine with castanospermine.

U.S. Pat. No. 4,925,835 to Heston discloses aziridinyl putrescine compounds such as 1-(4-aminobutyl)aziridine that can be used together with eflornithine.

U.S. Pat. No. 4,499,072 to Sunkara et al. discloses the use of eflornithine with interferon.

United States Patent Application Publication No. 2010/0076009 by Towner et al. discloses the use of eflornithine with 2,4-disulfonyl phenyl tert-butyl nitrone (2,4-ds-PBN) in the treatment of glioma.

United States Patent Application Publication No. 2015/0094336 by Zeldis discloses the use of eflornithine with 3-(4-amino-1-oxo-1,3-dihydro-isoindol-2-yl)-piperidine-2,6-dione or thalidomide in the treatment of glioma.

United States Patent Application Publication No. 2010/0285012 by Dunn, Jr. et al. discloses the use of eflornithine with N-2-pyridinyl-2-pyridinecarbothioamide or cambendazole in the treatment of glioma.

United States Patent Application Publication No. 2013/0197088 by Casero, Jr. et al. discloses the use of eflornithine with inhibitors of histone demethylase, including oligoamines and polyamines, for the treatment of malignancies.

United States Patent Application Publication No. 2015/0050299 by Burns et al. discloses the use of eflornithine together with one of a number of polyamine transport inhibitors, including AMXT 1426, AMXT 1501, AMXT 1505, and AMXT 1569.

United States Patent Application Publication No. 2008/0027023 by Ellervik et al. discloses the use of eflornithine together with aryl substituted xylopyranoside derivatives.

United States Patent Application Publication No. 2015/0017231 by Phanstiel, I V et al. discloses the use of eflornithine together with polyamine transport inhibitors with increased stability. The polyamine transport inhibitors are di-substituted aryl polyamine compounds with the structure R¹HN—(CH₂)ₓ—NH—(CH₂)_{y}—NH—CH₂—R—CH₂—NH—(CH₂)ₓₓ—NH—(CH₂)_{yy}—NHR″ wherein R is selected from the group consisting of anthracene, naphthalene, and benzene; wherein R′ and R″ are independently selected from the group consisting of H and an alkyl group; and wherein x, xx, y, and yy are independently selected from the group consisting of 3 and 4.

Eflornithine or derivatives or analogs thereof can also be used together with inhibitors of EGFR, particularly EGFR variant III (A. H. Thorne et al., “Epidermal Growth Factor Targeting and Challenges in Glioblastoma,” Neuro-Oncology 18: 914-918 (2016)). EGFR inhibitors include, but are not limited to, erlotinib, gefitinib, lapatinib, afatinib, dacomitinib, neratinib, and the monoclonal antibodies cetuximab, nimotuzumab, panitimumab, mAb425, ABT414, AMG595, and MR1-1. EGFR inhibitors that are monoclonal antibodies can be conjugated to additional therapeutically active agents, such as cytotoxins.

Additionally, eflornithine or a derivative or analog thereof, as described above, can be used with the following agents: bendamustine, carboplatin, carmustine (BCNU), cisplatin, cloretazine, diaziquone, fotemustine, lomustine (CCNU), melphalan, nimustine (ACNU), procarbazine, semustine, streptozotocin, temozolomide, teniposide, thalidomide, thioguanine, and vincristine. These agents include both alkylating agents and agents acting by different mechanisms.

Bendamustine (4-[5-[bis(2-chloroethyl)amino]-1-methylbenzimidazol-2-yl]butanoic acid) is an alkylating agent that is used for the treatment of malignancies including chronic lymphocytic leukemia, multiple myeloma, and non-Hodgkin's lymphoma. The structure of bendamustine is shown as Formula (A-I), below:

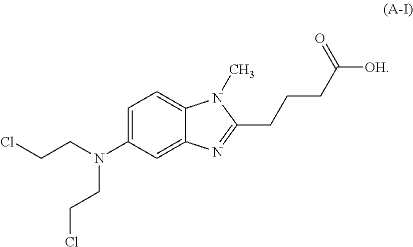 [SMILES: Cn1c(CCCC(=O)O)nc2cc(N(CCCl)CCCl)ccc21]

The use of bendamustine is disclosed in U.S. Pat. No. 8,436,190 to Brittain et al.

Carboplatin (cis-diammine(cyclobutane-1,1-dicarboxylate-O,O′)platinum (II)) is a platinum-containing DNA-damaging agent that is used for the treatment of ovarian cancer, lung cancer, head and neck cancer, brain cancer, and neuroblastoma.

Carboplatin is not an alkylating agent, although it has anti-neoplastic activities similar to those of alkylating agents. The structure of carboplatin is shown as Formula (A-III), below:

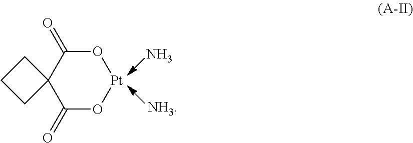

The use of carboplatin is disclosed in U.S. Pat. No. 5,104,896 to Nijkerk et al.

Carmustine (1,3-bis(2-chloroethyl)-1-nitrosourea) is an alkylating agent that is used to treat glioma, multiple myeloma, Hodgkin's lymphoma and non-Hodgkin's lymphoma. The structure of carmustine is shown as Formula (A-III), below:

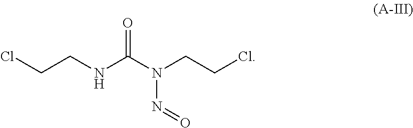 [SMILES: O=NN(CCCl)C(=O)NCCCl]

The use of carmustine is disclosed in U.S. Pat. No. 8,895,597 to Recinos et al.

Cisplatin (SP-4-2)-diamminedichloroplatinum (II)) is a platinum-containing DNA-damaging agent that is used for the treatment of testicular cancer, ovarian cancer, cervical cancer, breast cancer, bladder cancer, head and neck cancer, esophageal cancer, lung cancer, mesothelioma, brain tumors, and neoblastoma. Like carboplatin, cisplatin is not an alkylating agent, although it has anti-neoplastic activities similar to those of alkylating agents. The structure of cisplatin is shown as Formula (A-IV), below:

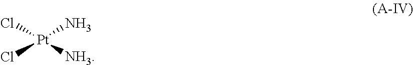

The use of cisplatin is disclosed in U.S. Pat. No. 4,889,724 to Kasan et al.

Cloretazine (also known as laromustine) (2-(2-chloroethyl)-N-methyl-1,2-bis(methylsulfonyl)hydrazinecarboxamide) is an alkylating agent that is proposed for use against acute myelocytic leukemia. The structure of cloretazine is shown as Formula (A-V):

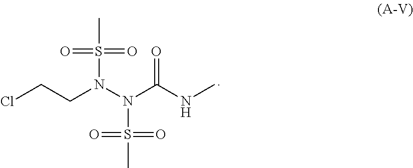 [SMILES: CNC(=O)N(N(CCCl)S(C)(=O)=O)S(C)(=O)=O]

The use of cloretazine is disclosed in U.S. Pat. No. 7,605,137 to King et al.

Diaziquone (2,5-diaziridinyl-3,6-bis(ethoxycarbonylamino)-1,4-benzoquinone) is an anti-neoplastic agent that may act as an alkylating agent and has shown broad antitumor activity against numerous transplantable murine tumors. The structure of diaziquone is shown as Formula (A-VI):

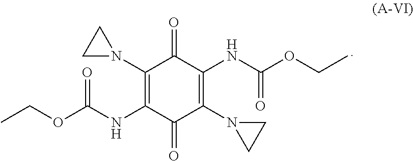 [SMILES: CCOC(=O)NC1=C(N2CC2)C(=O)C(NC(=O)OCC)=C(N2CC2)C1=O]

The use of diaziquone is disclosed in U.S. Pat. No. 9,943,519 to Folkes et al.

Fotemustine ((RS)-diethyl (1-{[(2-chloroethyl)(nitroso)carbamoyl]amino}ethyl)phosphonate) is a nitrosourea alkylating agent used for the treatment of metastatic melanoma. The structure of fotemustine is shown as Formula (A-VII):

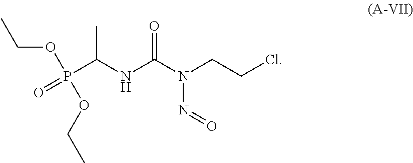 [SMILES: CCOP(=O)(OCC)C(C)NC(=O)N(CCCl)N=O]

The use of fotemustine is disclosed in U.S. Pat. No. 9,980,966 to Huck et al.

Lomustine (CCNU) (N-(2-chloroethyl)-N′-cyclohexyl-N-nitrosourea) is an alkylating agent that is a nitrosourea derivative and that is used for treatment of brain tumors and also as second-line therapy for Hodgkin's lymphoma. The structure of lomustine is shown as Formula (A-VIII):

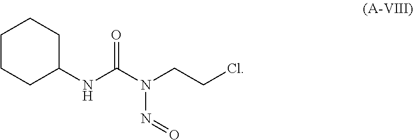 [SMILES: O=NN(CCCl)C(=O)NC1CCCCC1]

The use of lomustine is disclosed in U.S. Pat. No. 9,968,570 to Wanebo.

Melphalan ((2S)-2-amino-3-{4-[bis(2-chloroethyl)amino]phenyl}propanoic acid) is an alkylating agent of the nitrogen mustard class. Melphalan is used to treat multiple myeloma and ovarian cancer. The structure of melphalan is shown as Formula (A-IX):

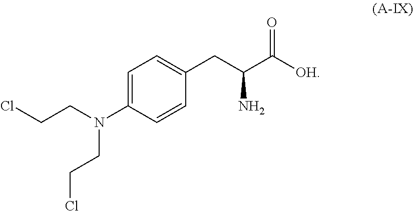 [SMILES: N[C@@H](Cc1ccc(N(CCCl)CCCl)cc1)C(=O)O]

The use of melphalan is disclosed in U.S. Pat. No. 4,738,843 to Oguchi et al.

Nimustine (ACNU) (N′-[(4-amino-2-methylpyrimidin-5-yl)methyl]-N-(2-chloroethyl)-N-nitrosourea) is a nitrosourea alkylating agent used for treatment of brain tumors. The structure of nimustine is shown as Formula (A-X):

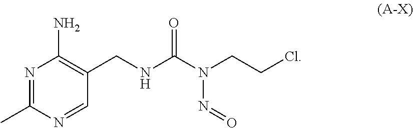 [SMILES: Cc1ncc(CNC(=O)N(CCCl)N=O)c(N)n1]

The use of nimustine is disclosed in U.S. Pat. No. 9,938,259 to Salituro et al.

Procarbazine (N-isopropyl-4-[(2-methylhydrazino)methyl]benzamide), also known as matulane, is an anti-neoplastic agent that damages DNA, most likely by its metabolism to azo-procarbazine and hydrogen peroxide, and is used for the treatment of Hodgkin's lymphoma and brain cancers. The structure of procarbazine is shown as Formula (A-XI):

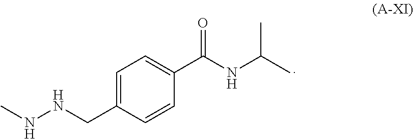 [SMILES: CNNCc1ccc(C(=O)NC(C)C)cc1]

The use of procarbazine is disclosed in U.S. Pat. No. 9,987,333 to Kangawa et al.

Semustine (N-(2-chloroethyl)-N′-(4-methylcyclohexyl)-N-nitrosourea) is a homologue of lomustine, being distinguished from it only by an additional methyl group. It has been proposed for use in rectal adenocarcinoma. The structure of semustine is shown below as Formula (A-XII):

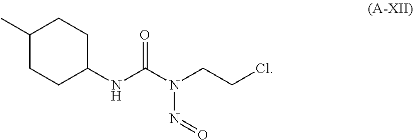 [SMILES: CC1CCC(NC(=O)N(CCCl)N=O)CC1]

The use of semustine is disclosed in U.S. Pat. No. 9,968,569 to Towner et al.

Streptozotocin (2-deoxy-2-({[methyl(nitroso)amino]carbonyl}amino)-β-D-glucopyran) is a glucosamine-nitrosourea derivative that is an alkylating agent and is used for treatment of pancreatic cancer. The structure of streptozotocin is shown below as Formula (A-XIII):

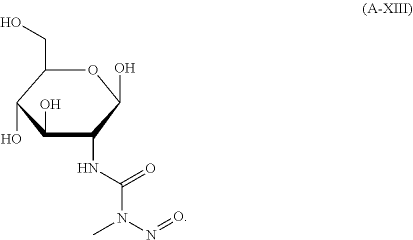

The use of streptozotocin is disclosed in U.S. Pat. No. 3,027,300 to Bergy et al.

Temozolomide (4-methyl-5-oxo-2,3,4,6,8-pentazabicyclo[4.3.0]nona-2,7,9-triene-9-carboxamide) is an alkylating agent that acts by alkylating the N⁷ or O⁶ positions of guanine residues. Temozolomide is used for treatment of nitrosourea-refractory or procarbazine-refractory anaplastic astrocytoma and newly diagnosed glioblastoma multiforme. It is a prodrug and an imidazotetrazine derivative of dacarbazine. The structure of temozolomide is shown below as Formula (A-XIV):

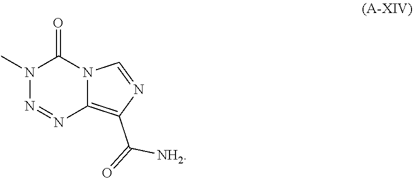

The use of temozolomide is disclosed in U.S. Pat. No. 6,346,524 to Ragab.

Teniposide ((5R,5aR,8aR,9S)-5,8,8a,9-tetrahydro-5-(4-hydroxy-3,5-dimethoxyphenyl)-9-({4,6-O—[(R)-2-thienylmethylene]-3-d-glucopyranosyl}oxy)furo[3′,4′:6,7]naphtho[2,3-d]-1,3-dioxol-6(5aH)-one) is an anti-neoplastic inhibitor of topoisomerase II that induces single- and double-strand breaks in DNA and is used for the treatment of acute lymphocytic leukemia, Hodgkin's lymphoma, reticulocyte sarcoma, glioblastoma, ependymoma, astrocytoma, bladder cancer, neuroblastoma, and other malignancies. The structure of teniposide is shown below as Formula (A-XIV):

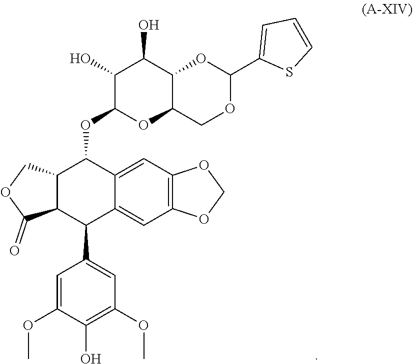

The use of teniposide is disclosed in U.S. Pat. No. 9,982,309 by Dang et al.

Thalidomide (2-(2,6-dioxopiperidin-3-yl)-2,3-dihydro-1H-isoindole-1,3-dione) is an anti-angiogenic anti-neoplastic agent used for the treatment of multiple myeloma. The structure of the (R)- and (S)-stereoisomers of thalidomide is shown below as Formula (A-XV):

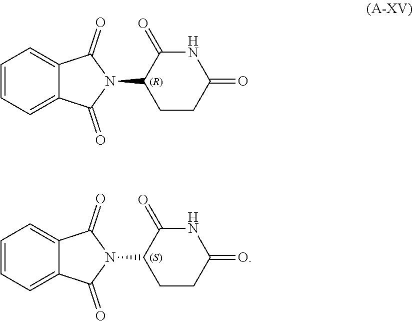

The use of thalidomide is for treatment of malignancies is disclosed in U.S. Pat. No. 9,006,267 by D'Angio et al.

Thioguanine (2-amino-1H-purine-6(7H)-thione) is a base analogue anti-neoplastic drug that is used for treatment of acute myeloid leukemia, acute lymphocytic leukemia, and chronic myeloid leukemia. The structure of thioguanine is shown below as Formula (A-XVI):

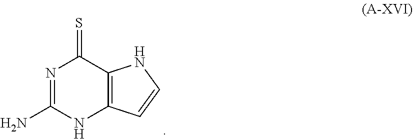 [SMILES: Nc1nc(=S)c2[nH]ccc2[nH]1]

The use of thioguanine is disclosed in U.S. Pat. No. 5,120,740 to Elfarra.

Vincristine (3aR,3a1R,4R,5S,5aR,10bR)-methyl 4-acetoxy-3a-ethyl-9-((5S,7S,9S)-5-ethyl-5-hydroxy-9-(methoxycarbonyl)-2,4,5,6,7,8,9,10-octahydro-1H-3,7-methano[1]azacycloundecino[5,4-b]indol-9-yl)-6-formyl-5-hydroxy-8-methoxy-3a,3a1,4,5,5a,6,11,12-octahydro-1H-indolizino[8,1-cd]carbazole-5-carboxylate) is an anti-neoplastic drug that binds to tubulin and inhibits mitosis, causing apoptosis and that is used for treatment of acute lymphocytic leukemia, acute myeloid leukemia, Hodgkin's lymphoma, neuroblastoma, and small cell lung cancer. The structure of vincristine is shown below as Formula (A-XVII):

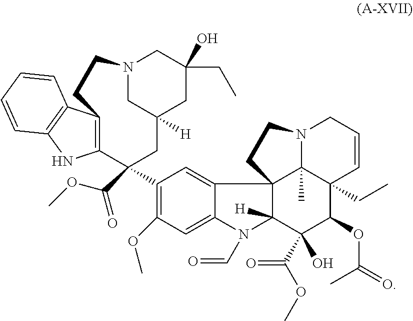

The use of vincristine is disclosed in U.S. Pat. No. 8,633,301 to Lejeune et al.

Suitable dosages, dosage frequencies, dosage durations, and routes of administration for these additional agents are known in the art. These additional agents can either be administered simultaneously with the eflornithine or the derivative or analog of eflornithine, or at a different time than the eflornithine or the derivative or analog of eflornithine. If the additional agent is administered at a different time than the eflornithine or the derivative or analog of eflornithine, it can either be administered before or after the eflornithine or the derivative or analog of eflornithine. One of ordinary skill in the art can determine a suitable schedule for administration based on variables such as the age, weight, and sex of the patient, the severity of the disease or condition for which the therapeutic agents are being administered, and pharmacokinetic parameters such as liver and kidney function.

The compounds described above can optionally be further substituted. In general, for optional substituents at saturated carbon atoms such as those that are part of the structures of the compounds described above, the following substituents can be employed: C₆-C₁₀ aryl, heteroaryl containing 1-4 heteroatoms selected from N, O, and S, C₁-C₁₀ alkyl, C₁-C₁₀ alkoxy, cycloalkyl, F, amino (NR¹R²), nitro, —SR, —S(O)R, —S(O₂)R, —S(O₂)NR¹R², and —CONR¹R², which can in turn be optionally substituted.

Further descriptions of potential optional substituents are provided below. The compounds that can be optionally substituted include: (i) eflornithine or derivatives or analogs thereof; (ii) other anti-neoplastic agents that can be used together with eflornithine or a derivative or analog thereof; and (iii) other agents that can potentiate the activity of eflornithine or a derivative or analog thereof, such as, but not limited to, inhibitors of polyamine transport.

Optional substituents as described above that are within the scope of the present invention do not substantially affect the activity of the derivative of the compound bearing one or more optional substituents or the stability of the derivative of the compound bearing one or more optional substituents, particularly the stability of the derivative in aqueous solution, and do not cause adverse interactions with other agents also used in methods according to the present invention. Definitions for a number of common groups that can be used as optional substituents are provided below; however, the omission of any group from these definitions cannot be taken to mean that such a group cannot be used as an optional substituent as long as the chemical and pharmacological requirements for an optional substituent are satisfied for the particular compound bearing the optional substituent.

As used herein, the term “alkyl” refers to an unbranched, branched, or cyclic saturated hydrocarbyl residue, or a combination thereof, of from 1 to 12 carbon atoms that can be optionally substituted; the alkyl residues contain only C and H when unsubstituted. Typically, the unbranched or branched saturated hydrocarbyl residue is from 1 to 6 carbon atoms, which is referred to herein as “lower alkyl.” When the alkyl residue is cyclic and includes a ring, it is understood that the hydrocarbyl residue includes at least three carbon atoms, which is the minimum number to form a ring. As used herein, the term “alkenyl” refers to an unbranched, branched or cyclic hydrocarbyl residue having one or more carbon-carbon double bonds. As used herein, the term “alkynyl” refers to an unbranched, branched, or cyclic hydrocarbyl residue having one or more carbon-carbon triple bonds; the residue can also include one or more double bonds. With respect to the use of “alkenyl” or “alkynyl,” the presence of multiple double bonds cannot produce an aromatic ring. As used herein, the terms “hydroxyalkyl,” “hydroxyalkenyl,” and “hydroxyalkynyl,” respectively, refer to an alkyl, alkenyl, or alkynyl group including one or more hydroxyl groups as substituents; as detailed below, further substituents can be optionally included. As used herein, the term “aryl” refers to a monocyclic or fused bicyclic moiety having the well-known characteristics of aromaticity; examples include phenyl, naphthyl, fluorenyl, and indenyl, which can be optionally substituted. As used herein, the term “hydroxyaryl” refers to an aryl group including one or more hydroxyl groups as substituents; as further detailed below, further substituents can be optionally included. As used herein, the term “heteroaryl” refers to monocyclic or fused bicylic ring systems that have the characteristics of aromaticity and include one or more heteroatoms selected from O, S, and N. The inclusion of a heteroatom permits aromaticity in 5-membered rings as well as in 6-membered rings. Typical heteroaromatic systems include monocyclic C₅-C₆ heteroaromatic groups such as pyridyl, pyrimidyl, pyrazinyl, thienyl, furanyl, pyrrolyl, pyrazolyl, thiazolyl, oxazolyl, triazolyl, triazinyl, tetrazolyl, tetrazinyl, and imidazolyl, as well as the fused bicyclic moieties formed by fusing one of these monocyclic heteroaromatic groups with a phenyl ring or with any of the heteroaromatic monocyclic groups to form a C₈-C₁₀ bicyclic group such as indolyl, benzimidazolyl, indazolyl, benzotriazolyl, isoquinolyl, quinolyl, benzothiazolyl, benzofuranyl, pyrazolylpyridyl, quinazolinyl, quinoxalinyl, cinnolinyl, and other ring systems known in the art. Any monocyclic or fused ring bicyclic system that has the characteristics of aromaticity in terms of delocalized electron distribution throughout the ring system is included in this definition. This definition also includes bicyclic groups where at least the ring that is directly attached to the remainder of the molecule has the characteristics of aromaticity, including the delocalized electron distribution that is characteristic of aromaticity. Typically the ring systems contain 5 to 12 ring member atoms and up to four heteroatoms, wherein the heteroatoms are selected from the group consisting of N, O, and S. Frequently, the monocyclic heteroaryls contain 5 to 6 ring members and up to three heteroatoms selected from the group consisting of N, O, and S; frequently, the bicyclic heteroaryls contain 8 to 10 ring members and up to four heteroatoms selected from the group consisting of N, O, and S. The number and placement of heteroatoms in heteroaryl ring structures is in accordance with the well-known limitations of aromaticity and stability, where stability requires the heteroaromatic group to be stable enough to be exposed to water at physiological temperatures without rapid degradation. As used herein, the term “hydroxyheteroaryl” refers to a heteroaryl group including one or more hydroxyl groups as substituents; as further detailed below, further substituents can be optionally included. As used herein, the terms “haloaryl” and “haloheteroaryl” refer to aryl and heteroaryl groups, respedively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. As used herein, the terms “haloalkyl,” “haloalkenyl,” and “haloalkynyl” refer to alkyl, alkenyl, and alkynyl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included.

As used herein, the term “optionally substituted” indicates that the particular group or groups referred to as optionally substituted may have no non-hydrogen substituents, or the group or groups may have one or more non-hydrogen substituents consistent with the chemistry and pharmacological activity of the resulting molecule. If not otherwise specified, the total number of such substituents that may be present is equal to the total number of hydrogen atoms present on the unsubstituted form of the group being described; fewer than the maximum number of such substituents may be present. Where an optional substituent is attached via a double bond, such as a carbonyl oxygen (C═O), the group takes up two available valences on the carbon atom to which the optional substituent is attached, so the total number of substituents that may be included is reduced according to the number of available valences. As used herein, the term “substituted,” whether used as part of “optionally substituted” or otherwise, when used to modify a specific group, moiety, or radical, means that one or more hydrogen atoms are, each, independently of each other, replaced with the same or different substituent or substituents.

Substituent groups useful for substituting saturated carbon atoms in the specified group, moiety, or radical include, but are not limited to, —Z^{a}, ═O, —OZ^{b}, —SZ^{b}, ═S—, —NZ^{C}Z^{c}, ═NZ^{b}, ═N—OZ^{b}, trihalomethyl, —CF₃, —CN, —OCN, —SCN, —NO, —NO₂, ═N₂, —N₃, —S(O)₂Z^{b}, —S(O)₂NZ^{b}, —S(O₂)O⁻, —S(O₂)OZ^{b}, —OS(O₂)OZ^{b}, —OS(O₂)O⁻, —OS(O₂)OZ^{b}, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)Z^{b}, —NZ^{b}C(S)Z^{b}, —NZ^{b}C(O)O⁻, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, —NZ^{b}C(NZ^{b})NZ^{c}Z^{c}, wherein Z^{a} is selected from the group consisting of alkyl, cycloalkyl, heteroalkyl, cycloheteroalkyl, aryl, arylalkyl, heteroaryl and heteroarylalkyl; each Z^{b} is independently hydrogen or Z^{a}; and each Z^{c} is independently Z^{b} or, alternatively, the two Z^{c}'s may be taken together with the nitrogen atom to which they are bonded to form a 4-, 5-, 6-, or 7-membered cycloheteroalkyl ring structure which may optionally include from 1 to 4 of the same or different heteroatoms selected from the group consisting of N, O, and S. As specific examples, —NZ^{c}Z^{c} is meant to include —NH₂, —NH-alkyl, —N-pyrrolidinyl, and —N-morpholinyl, but is not limited to those specific alternatives and includes other alternatives known in the art. Similarly, as another specific example, a substituted alkyl is meant to include -alkylene-O-alkyl, -alkylene-heteroaryl, -alkylene-cycloheteroaryl, -alkylene-C(O)OZ^{b}, -alkylene-C(O)NZ^{b}Z^{b}, and —CH₂—CH₂—C(O)—CH₃, but is not limited to those specific alternatives and includes other alternatives known in the art. The one or more substituent groups, together with the atoms to which they are bonded, may form a cyclic ring, including, but not limited to, cycloalkyl and cycloheteroalkyl.

Similarly, substituent groups useful for substituting unsaturated carbon atoms in the specified group, moiety, or radical include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —N₃, —S(O)₂Z^{b}, —S(O₂)O⁻, —S(O₂)OZ^{b}, —OS(O₂)OZ^{b}, —OS(O₂)O⁻, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

Similarly, substituent groups useful for substituting nitrogen atoms in heteroalkyl and cycloheteroalkyl groups include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —S(O)₂Z^{b}, —S(O₂)O⁻, —S(O₂)OZ^{b}, —OS(O₂)OZ^{b}, —OS(O₂)O⁻, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)OZ^{b}, —OC(S)OZ^{b}, —NZ^{b}C(O)Z^{b}, —NZ^{b}C(S)Z^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{C} are as defined above.

The compounds described herein may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms (such as the enantiomerically pure isomers, the E and Z isomers, and other alternatives for stereoisomers) as well as mixtures of stereoisomers in varying degrees of chiral purity or percentage of E and Z, including racemic mixtures, mixtures of diastereomers, and mixtures of E and Z isomers, unless a specific stereoisomer is specified. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The invention includes each of the isolated stereoisomeric forms as well as mixtures of stereoisomers in varying degrees of chiral purity, including racemic mixtures. It also encompasses the various diastereomers. Other structures may appear to depict a specific isomer, but that is merely for convenience, and is not intended to limit the invention to the depicted isomer. When the chemical name does not specify the isomeric form of the compound, it denotes any one of the possible isomeric forms or mixtures of those isomeric forms of the compound. As stated above, eflornithine exists in two enantiomeric forms.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium; the equilibrium may strongly favor one of the tautomers, depending on stability considerations. For example, ketone and enol are two tautomeric forms of one compound.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate.” Examples of hydrate include, but are not limited to, hemihydrate, monohydralte, dihydrate, trihydrate, hexahydrate, and other water-containing species.

It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

As used herein, the term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolysable esters of the present compounds are the compounds whose carboxyls are present in the form of hydrolysable ester groups. That is, these esters are pharmaceutically acceptable and can be hydrolyzed to the corresponding carboxyl acid in vivo.

In addition to the substituents described above, alkyl, alkenyl and alkynyl groups can alternatively or in addition be substituted by C₁-C₈ acyl, C₂-C₈ heteroacyl, C₆-C₁₀ aryl, C₃-C₈ cycloalkyl, C₃-C₈ heterocyclyl, or C₅-C₁₀ heteroaryl, each of which can be optionally substituted. Also, in addition, when two groups capable of forming a ring having 5 to 8 ring members are present on the same or adjacent atoms, the two groups can optionally be taken together with the atom or atoms in the substituent groups to which they are attached to form such a ring.

“Heteroalkyl,” “heteroalkenyl,” and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker.

Similarly, “heterocyclyl” may be used to describe a non-aromatic cyclic group that contains at least one heteroatom (typically selected from N, O and S) as a ring member and that is connected to the molecule via a ring atom, which may be C (carbon-linked) or N (nitrogen-linked); and “heterocyclylalkyl” may be used to describe such a group that is connected to another molecule through a linker. The heterocyclyl can be fully saturated or partially saturated, but non-aromatic. The sizes and substituents that are suitable for the cycloalkyl, cycloalkylalkyl, heterocyclyl, and heterocyclylalkyl groups are the same as those described above for alkyl groups. The heterocyclyl groups typically contain 1, 2 or 3 heteroatoms, selected from N, O and S as ring members; and the N or S can be substituted with the groups commonly found on these atoms in heterocyclic systems. As used herein, these terms also include rings that contain a double bond or two, as long as the ring that is attached is not aromatic. The substituted cycloalkyl and heterocyclyl groups also include cycloalkyl or heterocyclic rings fused to an aromatic ring or heteroaromatic ring, provided the point of attachment of the group is to the cycloalkyl or heterocyclyl ring rather than to the aromatic/heteroaromatic ring.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups.

Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C₇-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain.

In general, any alkyl, alkenyl, alkynyl, acyl, or aryl or arylalkyl group that is contained in a substituent may itself optionally be substituted by additional substituents. The nature of these substituents is similar to those recited with regard to the primary substituents themselves if the additional optional substituents are not otherwise described.

“Amino” as used herein refers to —NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle,” “carbocyclyl,” or “carbocyclic” refers to a cyclic ring containing only carbon atoms in the ring, whereas the term “heterocycle” or “heterocyclic” refers to a ring comprising a heteroatom. The carbocyclyl can be fully saturated or partially saturated, but non-aromatic. For example, the carbocyclyl encompasses cycloalkyl. The carbocyclic and heterocyclic structures encompass compounds having monocyclic, bicyclic or multiple ring systems; and such systems may mix aromatic, heterocyclic, and carbocyclic rings. Mixed ring systems are described according to the ring that is attached to the rest of the compound being described.

As used herein, the term “heteroatom” refers to any atom that is not carbon or hydrogen, such as nitrogen, oxygen or sulfur. When it is part of the backbone or skeleton of a chain or ring, a heteroatom must be at least divalent, and will typically be selected from N, O, P, and S.

As used herein, the term “alkanoyl” refers to an alkyl group covalently linked to a carbonyl (C═O) group. The term “lower alkanoyl” refers to an alkanoyl group in which the alkyl portion of the alkanoyl group is C₁-C₆. The alkyl portion of the alkanoyl group can be optionally substituted as described above. The term “alkylcarbonyl” can alternatively be used. Similarly, the terms “alkenylcarbonyl” and “alkynylcarbonyl” refer to an alkenyl or alkynyl group, respectively, linked to a carbonyl group.

As used herein, the term “alkoxy” refers to an alkyl group covalently linked to an oxygen atom; the alkyl group can be considered as replacing the hydrogen atom of a hydroxyl group. The term “lower alkoxy” refers to an alkoxy group in which the alkyl portion of the alkoxy group is C₁-C₆. The alkyl portion of the alkoxy group can be optionally substituted as described above. As used herein, the term “haloalkoxy” refers to an alkoxy group in which the alkyl portion is substituted with one or more halo groups.

As used herein, the term “sulfo” refers to a sulfonic acid (—SO₃H) substituent.

As used herein, the term “sulfamoyl” refers to a substituent with the structure —S(O₂)NH₂, wherein the nitrogen of the NH₂ portion of the group can be optionally substituted as described above.

As used herein, the term “carboxyl” refers to a group of the structure —C(O₂)H.

As used herein, the term “carbamyl” refers to a group of the structure —C(O₂)NH₂, wherein the nitrogen of the NH₂ portion of the group can be optionally substituted as described above.

As used herein, the terms “monoalkylaminoalkyl” and “dialkylaminoalkyl” refer to groups of the structure -Alk₁-NH-Alk₂ and -Alk₁-N(Alk₂)(Alk₃), wherein Alk₁, Alk₂, and Alk₃ refer to alkyl groups as described above.

As used herein, the term “alkylsulfonyl” refers to a group of the structure —S(O)₂-Alk wherein Alk refers to an alkyl group as described above. The terms “alkenylsulfonyl” and “alkynylsulfonyl” refer analogously to sulfonyl groups covalently bound to alkenyl and alkynyl groups, respectively. The term “arylsulfonyl” refers to a group of the structure —S(O)₂—Ar wherein Ar refers to an aryl group as described above.

The term “aryloxyalkylsulfonyl” refers to a group of the structure —S(O)₂-Alk-O—Ar, where Alk is an alkyl group as described above and Ar is an aryl group as described above. The term “arylalkylsulfonyl” refers to a group of the structure —S(O)₂-AlkAr, where Alk is an alkyl group as described above and Ar is an aryl group as described above.

As used herein, the term “alkyloxycarbonyl” refers to an ester substituent including an alkyl group wherein the carbonyl carbon is the point of attachment to the molecule. An example is ethoxycarbonyl, which is CH₃CH₂OC(O)—. Similarly, the terms “alkenyloxycarbonyl,” “alkynyloxycarbonyl,” and “cycloalkylcarbonyl” refer to similar ester substituents including an alkenyl group, alkenyl group, or cycloalkyl group respectively. Similarly, the term “aryloxycarbonyl” refers to an ester substituent including an aryl group wherein the carbonyl carbon is the point of attachment to the molecule. Similarly, the term “aryloxyalkylcarbonyl” refers to an ester substituent including an alkyl group wherein the alkyl group is itself substituted by an aryloxy group.

Other combinations of substituents are known in the art and, are described, for example, in U.S. Pat. No. 8,344,162 to Jung et al. For example, the term “thiocarbonyl” and combinations of substituents including “thiocarbonyl” include a carbonyl group in which a double-bonded sulfur replaces the normal double-bonded oxygen in the group. The term “alkylidene” and similar terminology refer to an alkyl group, alkenyl group, alkynyl group, or cycloalkyl group, as specified, that has two hydrogen atoms removed from a single carbon atom so that the group is double-bonded to the remainder of the structure.

The compounds disclosed herein may exist as salts at physiological pH ranges or other ranges. Such salts are described further below. In general, the term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isbutyric, oxalic, maleic, malonic, benzoic, succinic, suberic, fumeric, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, S. M. Berge, et al., “Pharmaceutical Salts,” J. Pharmaceutical Sci. 66: 1-19 (1977)). Certain specific compounds as used herein in methods and compositions according to the present invention contain both basic and acidic functionalities that allow the compounds to be converted into either base or acid addition salts.

Accordingly, one aspect of the present invention is a method of treating a glioma comprising the administration of a therapeutically effective quantity of eflornithine or a derivative or analog thereof to treat glioma by inhibiting progression of DNA mutations caused by chemotherapy agents to reduce the progression or grade of malignancy of the glioma. All pharmaceutically acceptable salt forms, hydrates, and solvates of eflornithine and derivatives, analogs, and prodrugs can be used in this method. Typically, the reduction of the rate of mutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16. More typically, the reduction of the rate of mutation is a reduction of the rate of mutation of at least one gene selected from the group consisting of H1FNT, RFX1, DHX36, MBD2, FRK, PEG3, NDC80, ABCA6, DNLZ, MUC12, PDCD6IP, and GSC2. However, the reduction of the rate of mutation is not limited to those genes, but also encompasses a large number of additional genes, as described below. In some alternatives according to the present invention, the effect of eflornithine or a derivative or analog thereof on mutation rate is assessed statistically over a large number of genes to determine an overall change in the mutation rate rather than focusing on specific genes.

In one alternative, the subject with glioma is currently treated with an alkylating agent. Typically, the alkylating agent is selected from the group consisting of temozolomide and lomustine. In another alternative, the alkylating agent can be, but is not limited to, an alkylating agent selected from the group consisting of: cyclophosphamide; mechlorethamine; uracil mustard; melphalan; chlorambucil; ifosfamide; bendamustine; carmustine; streptozotocin; busulfan; procarbazine; dacarbazine; mitocarbazine; altretamine; 6-methyluracil mustard; 6-ethyluracil mustard; 6-propyluracil mustard; 3-[1-methyl-4-[1-methyl-4-[1-methyl-4-[N1-[5-bis(2-chloroethyl)amino-2,4-(1H,3H)pyrimidinedione]acetylamino]-pyrrole-2-carboxamido]propionamidine hydrochloride; 3-[1-methyl-4-[1-methyl-4-[1-methyl-4-[N1-[5-bis(2-chloroethyl)amino-2,4-(1H,3H)pyrimidinedione]propanoylamino]-pyrrole-2-carboxamido]propionamidine hydrochloride; 3-[1-methyl-4-[1-methyl-4-[1-methyl-4-[N1-[5-bis(2-chloroethyl)amino-2,4-(1H,3H)pyrimidinedione]butanoylamino]-pyrrole-2-carboxamido]propionamidine hydrochloride; 3-[1-methyl-4-[1-methyl-4-[1-methyl-4-[N1-[5-bis(2-chloroethyl)amino-2,4-(1H,3H)pyrimidinedione]pentanoylamino]-pyrrole-2-carboxamido]propionamidine hydrochloride; 3-[1-methyl-4-[1-methyl-4-[1-methyl-4-[N1-[5-bis(2-chloroethyl)amino-2,4-(1H,3H)pyrimidinedione]hexanoylamino]-pyrrole-2-carboxamido]propionamidine hydrochloride; 3-[1-methyl-4-[1-methyl-4-[1-methyl-4-[N1-[5-bis(2-chloroethyl)amino-2,4-(1H,3H)pyrimidinedione]heptanoylamino]-pyrrole-2-carboxamido]propionamidine hydrochloride; estramustine; quinacrine mustard dihydrochloride; spiromustine; mustamine; phenylalanine mustard; mannomustine; 5-((bis(2-chloroethyl)amino)methyl)-pyrimidine-2,4(1H,3H)-dione; 5-((bis(2-chloroethyl)amino)methyl)-6-methylpyrimidine-2,4(1H,3H)-dione; 5-((bis(2-chloroethyl)amino)methyl)-1-methylpyrimidine-2,4(1H,3H)-dione; 5-((bis(2-chloroethyl)amino)methyl)-1,3-dimethylpyrimidine-2,4(1H,3H)-dione; 5-((bis(2-chloroethyl)amino)methyl)-6-propylpyrimidine-2,4(1H,3H)-dione; 5-((bis(2-chloroethyl)amino)methyl)-6-methyl-2-thioxo-2,3-dihydropyrimidin-4(1H)-one; nitrouracil; 5,6-dihydro-5-nitrouracil; 5,6-dihydro-5-nitro-1-(4-nitrophenyl)uracil; 5-nitro-1-(4-nitrophenyl)uracil; 5,6-dihydro-5-nitro-1(β-D-ribofuranuronic acid ethyl ester)uracil; 5-nitro-1(β-D-ribofuranuronic acid ethyl ester)uracil; 5-nitrouracil N-oxide; prednimustine; nimustine; ranimustine; fotemustine; ribomustine; cystemustine; 4-chlorouracil mustard; 4-cyanouracil mustard; 4-nitrouracil mustard; dianhydrogalactitol; diacetyldianhydrogalactitol; and dibromodulcitol.

In another alternative, the subject with glioma was previously treated with an alkylating agent. Typically, the interval between the previous treatment with the alkylating agent and the treatment with the eflornithine or derivative or analog thereof is from about 3 days to about 3 months. Other intervals can be employed.

In one alternative, the mutation is detected by DNA sequencing. Methods for DNA sequencing are known in the art. Methods for DNA sequencing include: the Maxam-Gilbert sequencing method involving chemical modification of DNA followed by cleavage at specific bases; the Sanger chain termination method; stepwise sequencing with removable 3′-blockers on DNA arrays; DNA colony sequencing involving random surface PCR-arraying methods; pyrosequencing; sequencing by synthesis; massively parallel signature sequencing; Polony sequencing; parallelized pyrosequencing; sequencing employing reversible dye-terminators; sequencing by use of rolling circle replication to amplify small fragments of DNA into DNA nanoballs; sequencing involving use of DNA fragments with added poly A tail adapters attached to a flow cell surface; nanopore DNA sequencing; sequencing employing tunneling currents; sequencing by hybridization; sequencing by mass spectroscopy; and sequencing employing RNA polymerase attached to polystyrene beads. Other DNA sequencing methods are known in the art. Other mutation analysis techniques are known in the art, including, but not limited to, restriction fragment length polymorphism (RFLP) analysis, terminal restriction fragment length polymorphism (TRFLP) analysis, cleaved amplified polymorphic sequence (CAPS) analysis, and other methods involving use of the polymerase chain reaction (PCR) procedure.

Typically, the eflornithine or derivative or analog thereof is selected from the group consisting of eflornithine and a pharmaceutically acceptable salt form, hydrate, or solvate thereof. Typically, when the eflornithine or derivative or analog thereof is eflornithine, as distinguished from a derivative or analog of eflornithine, the eflornithine is selected from the group consisting of: (i) a racemic mixture of D-eflornithine and L-eflornithine; (ii) D-eflornithine; and (iii) L-eflornithine.

In an alternative, the eflornithine or derivative or analog thereof is a derivative or analog of eflornithine. Such derivatives or analogs of eflornithine are as described above. Alternatively, the derivative or analog of eflornithine can be a derivative of such a derivative or analog that is, in turn, optionally substituted as described above, as long as the optional substituted derivative substantially retains the activity of the derivative or analog before optional substitution. As used herein, the term “substantially retains” is defined as having at least 80% of the inhibitory of ornithine decarboxylase on a molar basis when compared to eflornithine.

In one alternative, the eflornithine or derivative or analog thereof is administered orally or by injection.

In another alternative, the eflornithine or derivative or analog thereof is administered together with or adjuvant to radiotherapy.

Typically, the glioma being treated by administration of eflornithine or a derivative or analog thereof is characterized by one or more of the following characteristics:

(1) the glioma was previously treated with radiation therapy and adjuvant alkylator therapy and is recurrent/refractory anaplastic glioma;

(2) the glioma has a mutation in one or more genes selected from the group consisting of IDH1, IDH2, TP53, PTEN, ATRX, BRAF, CDKEN2A, SMARCA4, and PIK3;

(3) the glioma has the promoter for MGMT methylated; and

(4) the glioma has a mutation in at least one other gene that affects proliferation, survival, or resistance to chemotherapy.

In another alternative, the eflornithine or derivative or analog thereof is administered together with a therapeutically effective quantity of one or more conventional antineoplastic agents used for the treatment of glioma, wherein the one or more conventional antineoplastic agents are selected from the group consisting of: alkylating agents, platinum-containing agents that damage DNA, antimetabolites, anti-angiogenic agents, EGFR inhibitors, topoisomerase inhibitors, and tyrosine kinase inhibitors.

Alkylating agents include, but are not limited to, the agents described above. Typical alkylating agents used for the treatment of glioma include, but are not limited to, temozolomide and lomustine.

Antimetabolites include, but are not limited to: methotrexate, pemetrexed, 5-fluorouracil, capecitabine, cytarabine, gemcitabine, 6-mercaptopurine, and pentostatin, alanosine, AG2037 (Pfizer), 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrill-Dow DDFC, deazaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, piritrexim, plicamycin, Asahi Chemical PL-AC, Takeda TAC-788, thioguanine, tiazofurin, Erbamont TIF, trimetrexate, Taiho UFT and uricytin.

In another alternative, the eflornithine or derivative or analog thereof is administered together with an additional agent selected from the group consisting of:

(1) an inhibitor of polyamine transport;

(2) a polyamine analog;

(3) an S-adenosylmethionine decarboxylase inhibitor;

(4) an agent selected from the group consisting of: (a) a retinoid; (b) a syrbactin compound; (c) a cyclooxygenase-2 inhibitor; (d) a non-steroidal anti-inflammatory agent; (e) castanospermine or castanospermine esters; (f) an aziridinyl putrescine compound; (g) an interferon; (h) an aryl substituted xylopyranoside derivative; (i) an agent that reduces blood glutamate levels and enhances brain to blood glutamate efflux; (j) chitosan or chitosan derivatives and analogs; (k) 2,4-disulfonyl phenyl tert-butyl nitrone; (l) 3-(4-amino-1-oxo-1,3-dihydro-isoindol-2-yl)-piperidine-2,6-dione; (m) thalidomide; (n) N-2-pyridinyl-2-pyridinecarbothioamide; (o) cambendazole; and (p) an inhibitor of histone demethylase; and

(5) an agent that increases the ability of the eflornithine or derivative or analog thereof to pass through the blood-brain barrier.

In this aspect of the invention, the eflornithine or derivative or analog thereof is as described above for the treatment of glioma. Typically, the eflornithine or derivative or analog thereof is eflornithine as described above for the treatment of glioma. Typically, the eflornithine or derivative or analog thereof is administered orally or by injection.

In still another alternative, the eflornithine or derivative or analog thereof is administered together with or adjuvant to radiotherapy.

Another aspect of the invention is a pharmaceutical composition for the treatment of glioma comprising:

(1) a therapeutically effective quantity of eflornithine or a derivative or analog thereof, wherein the therapeutically effective quantity of eflornithine or the derivative or analog thereof reduces the rate of hypermutation of the glioma as a result of alkylating therapy exposure to reduce the progression of the glioma; and

(2) a pharmaceutically acceptable excipient. Typically, the reduction of the rate of hypermutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16.

In one alternative, wherein the composition is formulated for treatment of a subject with glioma, wherein the subject with glioma is selected from the group consisting of:
(i) a subject being currently treated with an alkylating agent;
(ii) a subject that had been previously treated with an alkylating agent;
(iii) a subject being currently treated with a platinum-containing antineoplastic agent that damages DNA;
(iv) a subject that had been previously treated with a platinum-containing antineoplastic agent that damages DNA; and
(v) a subject being currently or recently treated with radiotherapy.

Suitable candidates for administration of pharmaceutical compositions according to the present invention are as described above.

The eflornithine or derivative or analog of eflornithine included in the composition is as described above with respect to the treatment methods.

Typically, the composition is formulated for oral administration or administration by injection. When the composition is formulated for administration by injection, the administration by injection can be, but is not limited to, intravenous injection, intraperitoneal injection, subcutaneous injection, or intramuscular injection. The composition can be formulated for systemic administration or localized administration.

In another alternative, the composition further comprises a therapeutically effective quantity of one or more conventional antineoplastic agents used for the treatment of glioma, wherein the one or more conventional antineoplastic agents used for the treatment of glioma are selected from the group consisting of alkylating agents, platinum-containing agents that damage DNA, antimetabolites, anti-angiogenic agents, EGFR inhibitors, topoisomerase inhibitors, and tyrosine kinase inhibitors. Suitable alkylating agents, platinum-containing agents that damage DNA, antimetabolites, anti-angiogenic agents, EGFR inhibitors, topoisomerase inhibitors, and tyrosine kinase inhibitors are as described above.

In another alternative, the pharmaceutical composition can comprise a therapeutically effective quantity of an additional agent selected from the group consisting of:

(a) an inhibitor of polyamine transport;

(b) a polyamine analog;

(c) an S-adenosylmethionine decarboxylase inhibitor;

(d) an agent selected from the group consisting of: (1) a retinoid; (2) a syrbactin compound; (3) a cyclooxygenase-2 inhibitor; (4) a non-steroidal anti-inflammatory agent; (5) castanospermine or castanospermine esters; (6) an aziridinyl putrescine compound; (7) an interferon; (8) an aryl substituted xylopyranoside derivative; (9) an agent that reduces blood glutamate levels and enhances brain to blood glutamate efflux; (10) chitosan or chitosan derivatives and analogs; (11) 2,4-disulfonyl phenyl tert-butyl nitrone; (12) 3-(4-amino-1-oxo-1,3-dihydro-isoindol-2-yl)-piperidine-2,6-dione; (13) thalidomide; (14) N-2-pyridinyl-2-pyridinecarbothioamide; (15) cambendazole; and (16) an inhibitor of histone demethylase; and

(e) an agent that increases the ability of the eflornithine or derivative or analog thereof to pass through the blood-brain barrier.

Suitable inhibitors of polyamine transport include, but are not limited to, the agents described above.

Suitable polyamine analogs include, but are not limited to, the agents described above. These agents may also act as polyamine transport inhibitors.

Suitable retinoids include, but are not limited to, retinol, retinal, tretinoin (retinoic acid), isotretinoin, alitretinoin, etretinate, acitretin, adapalene, bexarotene, and tazarotene, 2-(nicotinamido)-ethyl retinoate, 2-(nicotinamido)-butyl retinoate, 5-(nicotinamido)-pentyl retinoate, 2-(nicotinamido)-hexyl retinoate, retinyl retinoate, retinyl palmitate and fenretinide, tamibarotene, retinoyl t-butyrate, retinoyl pinacol retinoyl cholesterol, retiferol, retinyl acetate, retinyl propionate, dehydroretinol, eretinate, eretrin, motretinide, retinoxytrimethylsilane, retinyl linoleate, and derivatives, analogs, and prodrugs thereof.

Suitable syrbactins include, but are not limited to, syringolins, glidobactins, and cepafungins.

Suitable cyclooxygenase-2 inhibitors include, but are not limited to, anitrafazen, apricoxib, celecoxib, cimicoxib, deracoxib, etoricoxib, firocoxib, flumizole, lumiracoxib, mavacoxib, N-[2-(cyclohexyloxy)-4-nitrophenyl]methanesulfonamide (NS-398), pamicogrel, parecoxib, polmacoxib, robenacoxib, rofecoxib, rutecarpine, tilmacoxib, and valdecoxib.

Suitable non-steroidal anti-inflammatory agents include, but are not limited to, in addition to cyclooxygenase-2 inhibitors, aspirin, aceclofenac, acemethacin, alclofenac, amoxiprin, ampyrone, azapropazone, benorylate, bromfenac, choline and magnesium salicylates, choline salicylate, clofezone, diclofenac potassium, diclofenac sodium, diclofenac sodium with misoprostol, diflunisal, droxicam, lornoxicam, meloxicam, tenoxicam, ethenzamide, etodolac, fenoprofen calcium, faislamine, flurbiprofen, flufenamic acid, ibuprofen, ibuproxam, indoprofen, alminoprofen, carprofen, dexibuprofen, dexketoprofen, fenbufen, flunoxaprofen, indomethacin, ketoprofen, ketorolac, kebuzone, loxoprofen, magnesium salicylate, meclofenamate sodium, metamizole, mofebutazone, oxyphenbutazone, phenazone, sulfinpyrazone, mefenamic acid, meloxicam, methyl salicylate, nabumetone, naproxen, naproxen sodium, nebumetone, oxaprozin, oxametacin, phenylbutazone, proglumetacin, piroxicam, pirprofen, suprofen, salsalate, salicyl salicylate, salicylamide, sodium salicylate, sulindac, tiaprofenic acid, tolfenamic acid, and tolmetin sodium.

Suitable agents that reduce blood glutamate levels and enhance brain to blood glutamate efflux include, but are not limited to, pyruvate, oxaloacetate, and lipoamide (Y. Li et al., “Scavenging of Blood Glutamate for Enhancing Brain-to-Blood Glutamate Efflux,” Mol. Med. Rep. 9: 305-310 (2014)).

Suitable histone demethylase inhibitors include, but are not limited to, ciclopirox, daminozide, N-[2-(2-pyridinyl)-6-(1,2,4,5-tetrahydro-3H-3-benzazepin-3-yl)-4-pyrimidinyl]-β-alanine, N-[2-(2-pyridinyl)-6-(1,2,4,5-tetrahydro-3H-3-benzazepin-3-yl)-4-pyrimidinyl]-β-alanine ethyl ester, rel-N-[(1 S,2R)-2-phenylcyclopropyl]-4-piperidinamine dihydrochloride, disodium (R)-2-dihydroxyglutarate, 8-hydroxy-5-quinolinecarboxylic acid, 5-chloro-2-[(E)-2-[phenyl(pyridin-2-yl)methylidene]hydrazin-1-yl]pyridine, 1,5-bis[(1E)-2-(3,4-dichlorophenyl)ethenyl]-2,4-dinitrobenzene, 2-(4-methylphenyl)-1,2-benzisothiazol-3(2H)-one, 1-(4-methyl-1-piperazinyl)-2-[[(1R*,2S*)-2-[4-phenylmethoxy)phenyl]cyclopropyl]amino]ethanone dihydrochloride, (1R,2S)-rel-2-[3,5-difluoro-2-(phenylmethoxy)phenyl]cyclopropanamine hydrochloride, and N-(9-cyclopropyl-1-oxononyl)-N-hydroxy-β-alanine. Other histone demethylase inhibitors are disclosed in U.S. Pat. No. 9,944,589 to Varasi et al. (cyclopropylamine derivatives); U.S. Pat. No. 9,908,865 to Nie et al. (substituted pyrazolylpyridine, pyrazolylpyridazine, and pyrazolylpyrimidine derivatives); U.S. Pat. No. 9,896,436 to Chen et al. (substituted imidazole-pyridine derivatives); U.S. Pat. No. 9,873,697 to Chen et al. (substituted pyrrolopyridine derivatives); U.S. Pat. No. 9,872,862 to Kuntz et al. (N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide); U.S. Pat. No. 9,834,550 to Kim et al. (pyridopyrimidone compounds); U.S. Pat. No. 9,828,343 to Chen et al. (substituted amidopyridine or amidopyridazine compounds); U.S. Pat. No. 9,815,828 to Boloor et al. (substituted pyridine derivatives); U.S. Pat. No. 9,745,384 to Shi et al. (peptides); U.S. Pat. No. 9,650,339 to Labelle et al. (compounds including a N-{[2-({[4-(diethylamino)butyl]amino}methyl)pyridin-4-yl] moiety); U.S. Pat. No. 9,643,965 to Boloor et al. (substituted pyrido[3,4-d]pyrimidin-4-one derivatives); U.S. Pat. No. 9,642,857 to Vankayalapati et al. (substituted (E)-N′-(1-phenylethylidene)benzohydrazide analogs and derivatives); U.S. Pat. No. 9,617,242 to Kanouni et al. (substituted 3-aminopyridine derivatives); U.S. Pat. No. 9,145,438 Chesworth et al. (7-deazapurines); U.S. Pat. No. 8,765,820 to Minucci et al. (tranylcypromine derivatives); and U.S. Pat. No. 8,735,622 to Wang et al. (compounds comprising a methyllysine mimic, a linker, and an α-ketoglutarate mimic). Other histone demethylase inhibitors are known in the art.

In one alternative of a composition according to the present invention, the quantity of the eflornithine or the derivative or analog of eflornithine in the composition is sufficient, in a unit dose, to modulate an immune response to the glioma.

In a composition according to the present invention, the pharmaceutically acceptable excipient can be selected from the group consisting of:
(i) a liquid carrier;
(ii) an isotonic agent;
(iii) a wetting or emulsifying agent;
(iv) a preservative;
(v) a buffer;
(vi) an acidifying agent;
(vii) an antioxidant;
(viii) an alkalinizing agent;
(ix) a carrying agent;
(x) a chelating agent;
(xi) a coloring agent;
(xii) a complexing agent;
(xiii) a solvent;
(xiv) a suspending and/or viscosity-increasing agent;
(xv) a flavor, perfume, or sweetening agent;
(xvi) an oil;
(xvii) a penetration enhancer;
(xviii) a polymer;
(xix) a stiffening agent;
(xx) a protein;
(xxi) a carbohydrate;
(xxii) a bulking agent; and
(xxiii) a lubricating agent.

Pharmaceutically acceptable excipients may be added to facilitate manufacture, enhance stability, control release, enhance product characteristics, enhance bioavailability, drug absorption or solubility, optimize other pharmacokinetic considerations, optimize the pharmaceutical formulation for a route of administration, enhance patient acceptability, or for another reason related to manufacture, storage, or use of a pharmaceutical composition. Excipients used in pharmaceutical compositions according to the present invention are compatible with the pharmaceutically active agent or agents included in the pharmaceutical composition, are compatible with other excipients included in the pharmaceutical composition, and are not injurious to and are tolerated by any patients to whom the pharmaceutical composition is administered. The particular excipient or excipients in any pharmaceutical composition according to the present invention can be varied according to such factors as the intended route of administration of the pharmaceutical composition, the quantity of eflornithine or a derivative or analog of eflornithine in a unit dose of the composition, the presence of other active agents such as those described above, including other anti-neoplastic agents, in the composition, and other factors generally understood in the art. Excipients for a pharmaceutical composition according to the present invention are selected such that they do not interfere with the activity of the eflornithine or derivative, analog, or prodrug thereof that is included in the pharmaceutical composition. Excipients for a pharmaceutical composition according to the present invention are also selected so that they do not interfere with the activity of other excipients or cause phase separation in the composition. In general, when a hydrophobic excipient such as an oil is included in the composition, a surfactant, wetting agent, or emulsifier is also included in the composition to ensure that phase separation does not occur and to ensure that composition remains stable and homogeneous. The quantities of any excipient included in a composition according to the present invention can be determined by one of ordinary skill in the art in order to ensure suitable physical properties of the composition and also in order to ensure suitable pharmacokinetics for the eflornithine or derivative, analog, or prodrug thereof included in the composition.

As is generally known in the art of pharmaceutical formulation, a particular excipient can fulfill one or more of these functions in a particular pharmaceutical composition, depending on the concentration of the excipient, the other excipients in the composition, the physical form of the composition, the concentration of active agent in the composition, the intended route of administration of the composition, and other factors. The recitation of a particular excipient in a category below is not intended to exclude the possible use of the excipient in another category or categories.

Typically, the liquid carrier can be, but is not limited to, a liquid carrier selected from the group consisting of saline, phosphate buffered saline, glycerol, and ethanol.

Typically, the isotonic agent can be, but is not limited to, a polyalcohol selected from the group consisting of mannitol and sorbitol, sodium chloride, and potassium chloride.

Typically, the wetting or emulsifying agent is a surfactant. Typically, the surfactant is selected from the group consisting of benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaureate, sorbitan monooleate, sorbitan monopalmitate, sorbitan monostearate, tyloxapol, acacia, cholesterol, diethanolamine, glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, soritan monooleate, sorbitan monopalmitate, sorbitan monostearate, stearic acid, triethanolamine, emulsifying wax, cetomacrogol, and cetyl alcohol.

Typically, the preservative is selected from the group consisting of benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, diazolidinyl urea, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, and thymol.

Typically, the buffer is selected from the group consisting of acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate, sodium bicarbonate, Tris (Tris(hydroxymethyl)aminomethane), MOPS (3-(N-morpholino)propanesulfonic acid), HEPES (N-(2-hydroxyethyl)piperazine-N′-(2-ethanesulfonic acid), ACES (2-[(2-amino-2-oxoethyl)amino]ethanesulfonic acid), ADA (N-(2-acetamido)2-iminodiacetic acid), AMPSO (3-[(1,1-dimethyl -2-hydroxyethylamino]-2-propanesulfonic acid), BES (N,N-bis(2-hydroxyethyl)-2-aminoethanesulfonic acid, Bicine (N,N-bis(2-hydroxyethylglycine), Bis-Tris (bis-(2-hydroxyethyl)imino-tris(hydroxymethyl)methane, CAPS (3-(cyclohexylamino)-1-propanesulfonic acid), CAPSO (3-(cyclohexylamino)-2-hydroxy-1-propanesulfonic acid), CHES (2-(N-cyclohexylamino)ethanesulfonic acid), DIPSO (3-[N,N-bis(2-hydroxyethylamino]-2-hydroxy-propanesulfonic acid), HEPPS (N-(2-hydroxyethylpiperazine)-N′-(3-propanesulfonic acid), HEPPSO (N-(2-hydroxyethyl)piperazine-N′-(2-hydroxypropanesulfonic acid), MES (2-(N-morpholino)ethanesulfonic acid), triethanolamine, imidazole, glycine, ethanolamine, phosphate, MOPSO (3-(N-morpholino)-2-hydroxypropanesulfonic acid), PIPES (piperazine-N,N′-bis(2-ethanesulfonic acid), POPSO (piperazine-N,N′-bis(2-hydroxypropaneulfonic acid), TAPS (N-tris[hydroxymethyl)methyl-3-aminopropanesulfonic acid), TAPSO (3-[N-tris(hydroxymethyl)methylamino]-2-hydroxy-propanesulfonic acid), TES (N-tris(hydroxymethyl)methyl-2-aminoethanesulfonic acid), tricine (N-tris(hydroxymethyl)methylglycine), 2-amino-2-methyl-1,3-propanediol, and 2-amino-2-methyl-1-propanol.

Typically, the acidifying agent is selected from the group consisting of acetic acid, citric acid, fumaric acid, hydrochloric acid, diluted hydrochloric acid, malic acid, nitric acid, phosphoric acid, diluted phosphoric acid, sulfuric acid, and tartaric acid.

Typically, the antioxidant is selected from the group consisting of ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite, sodium thiosulfate, sulfur dioxide, and tocopherol.

Typically, the alkalinizing agent is selected from the group consisting of strong ammonia solution, ammonium carbonate, diethanolamine, diisopropanolamine, potassium hydroxide, sodium bicarbonate, sodium borate, sodium carbonate, sodium hydroxide, and trolamine.

Typically, the chelating agent is selected from the group consisting of edetate disodium, ethylenediaminetetraacetic acid, citric acid, sodium 2,3-dimercapto-1-propanesulfonic acid, dimercaptosuccinic acid, metallothionein, desferroxamine, and salicylates.

Typically, the coloring agent is selected from the group consisting of ferric oxides red, yellow, black or blends, FD&C Red No. 3, FD&C Red No. 20, FD&C Yellow No. 6, FD&C Blue No. 2, D&C Green No. 5, D&C Orange No. 5, D&C Red No. 8, and dyes suitable for pharmaceutical use.

Typically, the complexing agent is selected from the group consisting of ethylenediaminetetraacetic acid, salts of ethylenediaminetetraacetic acid, gentisic acid ethanolamide, and oxyquinoline sulfate.

Typically, the solvent is selected from the group consisting of acetone, ethanol, diluted alcohol, amylene hydrate, benzyl benzoate, butyl alcohol, carbon tetrachloride, chloroform, corn oil, cottonseed oil, ethyl acetate, glycerol, hexylene glycol, isopropyl alcohol, methyl isobutyl ketone, mineral oil, oleic acid, peanut oil, polyethylene glycol, propylene carbonate, propylene glycol, sesame oil, water for injection, sterile water for injection, sterile water for irrigation, and purified water.

Typically, the suspending and/or viscosity-increasing agent is selected from the group consisting of acacia, agar, alginic acid, aluminum monostearate, bentonite, purified bentonite, magma bentonite, carbomers, carbomer 934p, carboxymethylcellulose calcium, carboxymethylcellulose sodium, carboxymethycellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, Veegum, and xanthan gum.

Typically, the flavor, perfume, or sweetening agent is selected from the group consisting of anise oil, cinnamon oil, menthol, anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin, aspartame, dextrates, dextrose, excipient dextrose, fructose, glycerol, mannitol, propylene glycol, saccharin, calcium saccharin, sodium saccharin, sorbitol, solution sorbitol, sucrose, compressible sugar, confectioner's sugar, and syrup.

Typically, the oil is selected from the group consisting of arachis oil, mineral oil, olive oil, sesame oil, cottonseed oil, safflower oil, corn oil, and soybean oil.

Typically, the penetration enhancer is selected from the group consisting of monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones, and ureas.

Typically, the polymer is selected from the group consisting of cellulose acetate, alkyl celluloses, hydroxyalkylcelluloses, acrylic polymers and copolymers, polyesters, polycarbonates, and polyanhydrides.

Typically, the stiffening agent is selected from the group consisting of hydrogenated castor oil, cetostearyl alcohol, cetyl alcohol, cetyl esters wax, hard fat, paraffin, polyethylene excipient, stearyl alcohol, emulsifying wax, white wax, and yellow wax.

Typically, the protein is selected from the group consisting of bovine serum albumin, human serum albumin (HSA), recombinant human albumin (rHA), gelatin, and casein.

Typically, the carbohydrate is selected from the group consisting of fructose, maltose, galactose, glucose, D-mannose, sorbose, lactose, sucrose, trehalose, cellobiose, raffinose, melezitose, maltodextrins, dextrans, starches, mannitol, maltitol, lactitol, xylitol, sorbitol, and myoinositol. As used herein, the term “carbohydrate” also encompasses carbohydrate derivatives such as sugar alcohols and also encompasses anomers of recited carbohydrates.

Typically, the bulking agent is selected from the group consisting of polypeptides and amino acids.

Typically, the lubricating agent is selected from the group consisting of magnesium stearate, stearic acid, sodium lauryl sulfate, and talc.

Other pharmaceutically acceptable excipients are known in the art and can be used in compositions according to the present application.

Oral dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms such as for example, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an enteric coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include croscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Enteric-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The eflornithine or derivative, analog or prodrug thereof can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes, colorings and flavors.

The active materials, such as eflornithine or a derivative, analog, or prodrug of eflornithine, can also be mixed with other active materials which do not impair the desired action, or with materials that supplement the desired action. The active ingredient is a compound or acceptable derivative thereof as described herein.

Higher concentrations, up to about 98% by weight of the active ingredient may be included.

In all embodiments of pharmaceutical compositions according to the present invention, tablets and capsules formulations may be coated as known by those of skill in the art in order to modify or sustain dissolution of the active ingredient. Thus, for example, they may be coated with a conventional enterically digestible coating, such as phenyl salicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Vehicles used in elixirs include solvents. Syrups are concentrated aqueous solutions of a sugar, for example, sucrose, and may contain a preservative. An emulsion is a two-phase system in which one liquid is dispersed in the form of small globules throughout another liquid. Vehicles used in emulsions are non-aqueous liquids, emulsifying agents and preservatives. Suspensions use suspending agents and preservatives. Substances used in non-effervescent granules, to be reconstituted into a liquid oral dosage form, include diluents, sweeteners and wetting agents. Substances used in effervescent granules, to be reconstituted into a liquid oral dosage form, include organic acids and a source of carbon dioxide. Coloring and flavoring agents can be used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite and surfactants such as polyoxyethylene sorbitan monooleate.

Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such as fruits, and synthetic blends of compounds which produce a pleasant taste sensation.

For a solid dosage form, the solution or suspension, in for example, propylene carbonate, vegetable oils or triglycerides, is in some embodiments encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a liquid vehicle, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including an acetal. Alcohols used in these formulations are any water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

Another aspect of the present invention is a method for the treatment of a malignancy selected from the group consisting of a non-glioma central nervous system malignancy, colorectal cancer, leukemia, lymphoma, pancreatic cancer, liver cancer, and stomach cancer comprising the step of administering a therapeutically effective quantity of eflornithine or a derivative or analog thereof to a subject with the malignancy in order to reduce the rate of hypermutation of the malignancy to reduce the progression of the malignancy, wherein the reduction of the rate of hypermutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16. Typically, the mutation of at least one gene is selected from the group consisting of H1FNT, RFX1, DHX36, MBD2, FRK, PEG3, NDC80, ABCA6, DNLZ, MUC12, PDCD6IP, and GSC2. Pharmaceutical compositions as described above can be used in this method for treatment of such malignancies.

In one alternative, the composition is formulated for treatment of a subject with the malignancy, wherein the subject with the malignancy is selected from the group consisting of:
(i) a subject being currently treated with an alkylating agent;
(ii) a subject that had been previously treated with an alkylating agent;
(iii) a subject being currently treated with a platinum-containing antineoplastic agent that damages DNA;
(iv) a subject that had been previously treated with a platinum-containing antineoplastic agent that damages DNA; and
(v) a subject being currently or recently treated with radiotherapy.

In this embodiment of a composition according to the present invention, the eflornithine or derivative or analog thereof is as described above.

As with the embodiment of the composition disclosed above for treatment of glioma, the composition can be formulated for oral administration or administration by injection. When the composition is formulated for administration by injection, the administration by injection can be, but is not limited to, intravenous injection, intraperitoneal injection, subcutaneous injection, or intramuscular injection. The composition can be formulated for systemic administration or localized administration.

This embodiment of the pharmaceutical composition can further comprise a therapeutically effective quantity of one or more conventional antineoplastic agents used for the treatment of the malignancy, wherein the one or more conventional antineoplastic agents used for the treatment of the malignancy are selected from the group consisting of alkylating agents, platinum-containing agents that damage DNA, antimetabolites, anti-angiogenic agents, EGFR inhibitors, topoisomerase inhibitors, and tyrosine kinase inhibitors.

Additionally, this embodiment of the pharmaceutical composition can further comprise a therapeutically effective quantity of an additional agent as described above, such as, for example, an inhibitor of polyamine transport or a polyamine analog.

In this embodiment of the pharmaceutical composition, the quantity of the eflornithine or the derivative or analog of eflornithine in the composition can be sufficient, in a unit dose, to modulate an immune response to the malignancy. This embodiment of the pharmaceutical composition can, alternatively, further comprise a therapeutically effective quantity of one or more immunomodulatory agents used for the treatment of the malignancy. Suitable immunomodulatory agents are as described above.

In this embodiment of the pharmaceutical composition, suitable pharmaceutically acceptable excipients are as described above.

In yet another embodiment of a pharmaceutical composition according to the present invention, the composition comprises:

(1) a therapeutically effective quantity of eflornithine or a derivative or analog thereof, wherein the therapeutically effective quantity of eflornithine or the derivative or analog thereof reduces the rate of mutation of the glioma to reduce the progression of the glioma, wherein the reduction of the rate of mutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16;

(2) a therapeutically effective quantity of an alkylating agent; and

(3) a pharmaceutically acceptable excipient.

In this embodiment of a pharmaceutical composition according to the present invention, the composition can be formulated for treatment of a subject with glioma, wherein the subject with glioma is selected from the group consisting of:
(i) a subject being currently treated with an alkylating agent;
(ii) a subject that had been previously treated with an alkylating agent;
(iii) a subject being currently treated with a platinum-containing antineoplastic agent that damages DNA;
(iv) a subject that had been previously treated with a platinum-containing antineoplastic agent that damages DNA; and
(v) a subject being currently or recently treated with radiotherapy.

In this embodiment of a composition according to the present invention, the eflornithine or derivative or analog thereof is as described above.

As with the embodiments of the pharmaceutical composition disclosed above, the composition can be formulated for oral administration or administration by injection. When the composition is formulated for administration by injection, the administration by injection can be, but is not limited to, intravenous injection, intraperitoneal injection, subcutaneous injection, or intramuscular injection. The composition can be formulated for systemic administration or localized administration.

Typically, the alkylating agent is selected from the group consisting of lomustine and temozolomide. Alternatively, the alkylating agent can be another alkylating agent known in the art and usable for treatment of glioma, with or without additional agents.

This embodiment of a pharmaceutical composition according to the present invention can further comprise a therapeutically effective quantity of one or more conventional antineoplastic agents used for the treatment of glioma as described above. This embodiment of a pharmaceutical composition according to the present invention can, alternatively, further comprise a therapeutically effective quantity of an additional agent, such as an inhibitor of polyamine transport, as described above.

In this embodiment of a pharmaceutical composition according to the present invention, the quantity of the eflornithine or the derivative or analog of eflornithine in the composition can be sufficient, in a unit dose, to modulate an immune response to the glioma.

In another alternative, this embodiment of a pharmaceutical composition according to the present invention can further comprise a therapeutically effective quantity of a therapeutically effective quantity of one or more immunomodulatory agents used for the treatment of glioma, wherein the immunomodulatory agents are as described above.

Typically, in this embodiment of a pharmaceutical composition according to the present invention, the alkylating agent is selected from the group consisting of lomustine and temozolomide.

In an alternative, in this embodiment of a pharmaceutical composition according to the present invention, the alkylating agent can be, but is not limited to, an alkylating agent selected from the group consisting of: cyclophosphamide; mechlorethamine; uracil mustard; melphalan; chlorambucil; ifosfamide; bendamustine; carmustine; streptozotocin; busulfan; procarbazine; dacarbazine; mitocarbazine; altretamine; 6-methyluracil mustard; 6-ethyluracil mustard; 6-propyluracil mustard; 3-[1-methyl-4-[1-methyl-4-[1-methyl-4-[N1-[5-bis(2-chloroethyl)amino-2,4-(1H,3H)pyrimidinedione]acetylamino]-pyrrole-2-carboxamido]propionamidine hydrochloride; 3-[1-methyl-4-[1-methyl-4-[1-methyl-4-[N1-[5-bis(2-chloroethyl)amino-2,4-(1H,3H)pyrimidinedione]propanoylamino]-pyrrole-2-carboxamido]propionamidine hydrochloride; 3-[1-methyl-4-[1-methyl-4-[1-methyl-4-[N1-[5-bis(2-chloroethyl)amino-2,4-(1H,3H)pyrimidinedione]butanoylamino]-pyrrole-2-carboxamido]propionamidine hydrochloride; 3-[1-methyl-4-[1-methyl-4-[1-methyl-4-[N1-[5-bis(2-chloroethyl)amino-2,4-(1H,3H)pyrimidinedione]pentanoylamino]-pyrrole-2-carboxamido]propionamidine hydrochloride; 3-[1-methyl-4-[1-methyl-4-[1-methyl-4-[N1-[5-bis(2-chloroethyl)amino-2,4-(1H,3H)pyrimidinedione]hexanoylamino]-pyrrole-2-carboxamido]propionamidine hydrochloride; 3-[1-methyl-4-[1-methyl-4-[1-methyl-4-[N1-[5-bis(2-chloroethyl)amino-2,4-(1H,3H)pyrimidinedione]heptanoylamino]-pyrrole-2-carboxamido]propionamidine hydrochloride; estramustine; quinacrine mustard dihydrochloride; spiromustine; mustamine; phenylalanine mustard; mannomustine; 5-((bis(2-chloroethyl)amino)methyl)-pyrimidine-2,4(1H,3H)-dione; 5-((bis(2-chloroethyl)amino)methyl)-6-methylpyrimidine-2,4(1H,3H)-dione; 5-((bis(2-chloroethyl)amino)methyl)-1-methylpyrimidine-2,4(1H,3H)-dione; 5-((bis(2-chloroethyl)amino)methyl)-1,3-dimethylpyrimidine-2,4(1H,3H)-dione; 5-((bis(2-chloroethyl)amino)methyl)-6-propylpyrimidine-2,4(1H,3H)-dione; 5-((bis(2-chloroethyl)amino)methyl)-6-methyl-2-thioxo-2,3-dihydropyrimidin-4(1H)-one; nitrouracil; 5,6-dihydro-5-nitrouracil; 5,6-dihydro-5-nitro-1-(4-nitrophenyl)uracil; 5-nitro-1-(4-nitrophenyl)uracil; 5,6-dihydro-5-nitro-1(β-D-ribofuranuronic acid ethyl ester)uracil; 5-nitro-1(β-D-ribofuranuronic acid ethyl ester)uracil; 5-nitrouracil N-oxide; prednimustine; nimustine; ranimustine; fotemustine; ribomustine; cystemustine; 4-chlorouracil mustard; 4-cyanouracil mustard; 4-nitrouracil mustard; dianhydrogalactitol; diacetyldianhydrogalactitol; and dibromodulcitol.

Suitable excipients for this embodiment of a composition according to the present invention are as described above.

Yet another aspect of the present invention is a pharmaceutical composition for the treatment of a malignancy selected from the group consisting of a non-glioma central nervous system malignancy, colorectal cancer, leukemia, lymphoma, pancreatic cancer, liver cancer, and stomach cancer comprising:

(1) a therapeutically effective quantity of eflornithine or a derivative or analog thereof, wherein the therapeutically effective quantity of eflornithine or the derivative or analog thereof reduces the rate of mutation of the malignancy to reduce the progression of the malignancy, wherein the reduction of the rate of mutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16; and

(2) a pharmaceutically acceptable excipient.

In one alternative, in this embodiment of a pharmaceutical composition according to the present invention, the composition is formulated for treatment of a subject with the malignancy, wherein the subject with the malignancy is selected from the group consisting of:
(i) a subject being currently treated with an alkylating agent;
(ii) a subject that had been previously treated with an alkylating agent;
(iii) a subject being currently treated with a platinum-containing antineoplastic agent that damages DNA;
(iv) a subject that had been previously treated with a platinum-containing antineoplastic agent that damages DNA; and
(v) a subject being currently or recently treated with radiotherapy.

In this embodiment of a pharmaceutical composition according to the present invention, the eflornithine or derivative or analog thereof, the alkylating agent, and the pharmaceutically acceptable excipient are as described above. The pharmaceutical composition can be formulated for administration as described above. In various alternatives, the composition can: (i) further comprise a therapeutically effective quantity of one or more conventional antineoplastic agents used for the treatment of the malignancy as described above; (ii) further comprise a therapeutically effective quantity of an additional agent, such as an inhibitor of polyamine transport, as described above; (iii) include a quantity of the eflornithine or the derivative or analog of eflornithine in the composition that is sufficient, in a unit dose, to modulate an immune response to the malignancy; or (iv) further comprise a therapeutically effective quantity of a therapeutically effective quantity of one or more immunomodulatory agents used for the treatment of the malignancy as described above.

Yet another aspect of the present invention is a kit comprising, separately packaged:

(1) a therapeutically effective quantity of eflornithine or a derivative or analog thereof, wherein the quantity of the eflornithine or the derivative or analog thereof is a therapeutically effective quantity for treatment of a glioma such that the rate of mutation of the glioma is reduced to reduce the progression of the glioma, wherein the reduction of the rate of mutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16;

(2) a therapeutically effective quantity of an alkylating agent for treatment of the glioma; and

(3) instructions for use of the kit.

The kit can further comprise, separately packaged, a pharmaceutically acceptable excipient to be combined with the therapeutically effective quantity of the eflornithine or the derivative or analog thereof. Suitable excipients are as described above. The kit can also further comprise, separately packaged, a pharmaceutically acceptable excipient to be combined with the therapeutically effective quantity of the alkylating agent. Suitable excipients are as described above. In the kit, alternatives for the eflornithine or derivative or analog thereof and the alkylating agent are as described above. The kit can be formulated so that the eflornithine or derivative or analog thereof is administered orally or by injection.

Yet another aspect of the present invention is a kit comprising, separately packaged:

(1) a therapeutically effective quantity of eflornithine or a derivative or analog thereof, wherein the quantity of the eflornithine or the derivative or analog thereof is a therapeutically effective quantity for treatment of a malignancy selected from the group consisting of a non-glioma central nervous system malignancy, colorectal cancer, leukemia, lymphoma, pancreatic cancer, liver cancer, and stomach cancer such that the rate of mutation of the glioma is reduced to reduce the progression of the malignancy, wherein the reduction of the rate of mutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16;

(2) a therapeutically effective quantity of an alkylating agent for treatment of the malignancy; and

(3) instructions for use of the kit.

The kit can further comprise, separately packaged, a pharmaceutically acceptable excipient to be combined with the therapeutically effective quantity of the eflornithine or the derivative or analog thereof, or, separately packaged, a pharmaceutically acceptable excipient to be combined with the therapeutically effective quantity of the alkylating agent. Suitable pharmaceutically acceptable excipients are as stated above. In the kit, alternatives for the eflornithine or derivative or analog thereof and the alkylating agent are as described above. The kit can be formulated so that the eflornithine or derivative or analog thereof is administered orally or by injection.

As stated previously, the eflornithine or derivative or analog thereof can be administered alone or together with a therapeutically effective quantity of one or more conventional anti-neoplastic agents used for the treatment of glioma, or, alternatively, used for treatment of a malignancy other than glioma as described above. These agents can include, but are not limited to, alkylating agents, antimetabolites, anti-angiogenic agents, EGFR inhibitors, platinum-containing agents, topoisomerase inhibitors, or other classes of agents. Particular combinations of additional agents can also be used. For example, but not by way of limitation, these additional agents or combinations of additional agents can include lomustine (CCNU), carmustine (BCNU), temozolomide, procarbazine, vincristine, PCV (a combination of lomustine, procarbazine, and vincristine), carboplatin, carboplatin plus thymidine, carmustine plus temozolomide, erlotinib, carboplatin plus erlotinib, cloretazine, lomustine plus cloretazine, imatinib, hydroxyurea, hydroxyurea plus imatinib, irinotecan, thalidomide, temozolomide plus thalidomide, rilotumumab, cilengitide, cis-retinoic acid, celecoxib, cis-retinoic acid plus celecoxib, enzastaurin, sirolimus, erlotinib plus sirolimus, fenretinide, gefitinib, lapatinib, temsirolimus, tipifarnib, vorinostat, diaziquone, methotrexate, melphalan, a combination such as TPDCV (thioguanine, procarbazine, dibromodulcitol, lomustine, vincristine), a combination of nitrogen mustard, vincristine, and procarbazine, teniposide, and carboplatin plus teniposide. Other agents and combinations of agents are known in the art and are within the scope of the present invention.

Eflornithine or a derivative or analog thereof, either alone or together with one or more additional agents as described above, can also be used together with radiotherapy. Similarly, eflornithine or a derivative or analog thereof, plus agents such as: polyamine analogs; polyamine transport inhibitors; S-adenosylmethionine decarboxylase inhibitor; agents selected from the group consisting of: (1) a retinoid; (2) a syrbactin compound; (3) a cyclooxygenase-2 inhibitor; (4) a non-steroidal anti-inflammatory agent; (5) castanospermine or castanospermine esters; (6) an aziridinyl putrescine compound; (7) an interferon; (8) an aryl substituted xylopyranoside derivative; (9) an agent that reduces blood glutamate levels and enhances brain to blood glutamate efflux; (10) chitosan or chitosan derivatives and analogs; (11) 2,4-disulfonyl phenyl tert-butyl nitrone; (12) 3-(4-amino-1-oxo-1,3-dihydro-isoindol-2-yl)-piperidine-2,6-dione; (13) thalidomide; (14) N-2-pyridinyl-2-pyridinecarbothioamide; (15) cambendazole; and (16) an inhibitor of histone demethylase; agents that increase the the ability of the eflornithine or derivative or analog thereof to pass through the blood-brain barrier; an agent or method selected from the group consisting of: (1) passive immunotherapy with ex vivo activation of immune cells; (2) immunotherapy with chimeric antigen receptors; (3) ex vivo loading of dendritic cells with tumor antigens followed by reintroduction of the dendritic cells loaded with tumor antigens; (4) use of a vaccine targeting the IL-13 zetakine (IL13Ra2); (5) use of in situ gene therapy with Ad-Flt3L and Ad-Tk; (6) use of cancer stem cell antigens for vaccination; (7) use of macrophages loaded with gold-coated nanoshells in connection with photothermal therapy; (8) use of peripheral blood mononuclear cells that are collected and genetically modified to express the membrane-tethered IL-13 cytokine chimeric T cell receptor targeting the IL-13 receptor a2 (IL13Ra2); and (9) use of vaccine therapy with autologous dendritic cells; and an immunomodulatory agent selected from the group consisting of (1) IL-15; (2) anti-PD1 antibodies; (3) anti-B7-H1 antibodies; (4) IL-12; (5) QS-21; (6) CD-40; (7) anti-CD40 antibody acting as a CD40 agonist; (8) CD40L; (9) IL-7; (10) CpG; (11) 1-methyltryptophan; (12) anti-CD137 antibodies; (13) anti-TGF-0 antibodies; (14) anti-IL10 antibodies; (15) anti-ILR10R antibodies; (16) Flt3L; (17) anti-GITR; (18) CCL21 or a nucleic acid encoding CCL21; (19) monophosphoryl lipid A; (20) poly I:C; (21) poly ICLC; (22) anti-OX40 antibodies; (23) anti-B7-H4 antibodies; (24) an immune response modulator selected from the group consisting of: resiquimod; N-[4-(4-amino-2-ethylimidazo[4,5-c]quinolin-1-yl)butyl]methanesulfonamide); imiquimod; 2-ethoxymethyl)-1H-imidazo[4,5-c]quinolin-4-amine; 2-propylthiazolo[4,5-c]quinolin-4-amine; isatoribine; ANA975, ANA-773; and GS-9620; (25) LIGHT or a nucleic acid encoding LIGHT; (26) antibodies to LAG-3; and (27) antibodies to CTLA4, can be administered together with radiotherapy.

In another alternative as described above, the eflornithine or derivative or analog thereof is administered together with an agent that increases the ability of the eflornithine or derivative or analog thereof to pass through the blood-brain barrier, particularly for treatment of gliomas or other central nervous system malignancies. Typically, the agent that increases the ability of the eflornithine or derivative or analog thereof to pass through the blood-brain barrier is an agent selected from the group consisting of:
(a) a chimeric peptide of the structure of Formula (D-III):

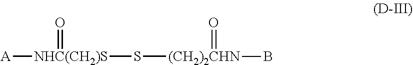

wherein: (A) A is somatostatin, thyrotropin releasing hormone (TRH), vasopressin, alpha interferon, endorphin, muramyl dipeptide or ACTH 4-9 analogue; and (B) B is insulin, IGF-I, IGF-II, transferrin, cationized (basic) albumin or prolactin; or a chimeric peptide of the structure of Formula (D-III) wherein the disulfide conjugating bridge between A and B is replaced with a bridge of Subformula (D-III(a)):

A-NH(CH₂)₂S—S—B(cleavable linkage) (D-III(a)),

wherein the bridge is formed using cysteamine and EDAC as the bridge reagents; or a chimeric peptide of the structure of Formula (D-III) wherein the disulfide conjugating bridge between A and B is replaced with a bridge of Subformula (D-III(b)):

A-NH—CH(CH₂)₃CH═NH—B(non-cleavable linkage) (D-III(b)),

wherein the bridge is formed using glutaraldehyde as the bridge reagent;
(b) a composition comprising either avidin or an avidin fusion protein bonded to a biotinylated eflornithine or analog or derivative thereof to form an avidin-biotin-agent complex including therein a protein selected from the group consisting of insulin, transferrin, an anti-receptor monoclonal antibody, a cationized protein, and a lectin;
(c) a neutral liposome that is pegylated and incorporates the eflornithine or analog or derivative thereof, wherein the polyethylene glycol strands are conjugated to at least one transportable peptide or targeting agent;
(d) a humanized murine antibody that binds to the human insulin receptor linked to the eflornithine or analog or derivative thereof through an avidin-biotin linkage; and
(e) a fusion protein comprising a first segment and a second segment: the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and the second segment comprising a protein domain selected from the group consisting of avidin, an avidin mutein, a chemically modified avidin derivative, streptavidin, a streptavidin mutein, and a chemically modified streptavidin derivative, wherein the fusion protein is linked to the eflornithine or analog or derivative thereof by a covalent link to biotin.

Still another aspect of the present invention is a method for treatment of a malignancy, such as a glioma, comprising the step of administering: (1) a therapeutically effective quantity of eflornithine or a derivative or analog of eflornithine; and (2) a therapeutically effective quantity of an immunomodulatory agent selected from the group consisting of: (a) IL-15; (b) anti-PD1 antibodies; (c) anti-B7-H1 antibodies; (d) IL-12; (e) QS-21; (f) CD-40; (g) anti-CD40 antibody acting as a CD40 agonist; (h) CD40L; (i) IL-7; (j) CpG; (k) 1-methyltryptophan; (l) anti-CD137 antibodies; (m) anti-TGF-[3 antibodies; (n) anti-IL10 antibodies; (o) anti-ILR1R antibodies; (p) Flt3L; (q) Anti-GITR; (r) CCL21 or a nucleic acid encoding CCL21; (s) monophosphoryl lipid A; (t) poly I:C; (u) poly ICLC; (v) anti-OX40 antibodies; (w) anti-B7-H4 antibodies; (x) an immune response modulator selected from the group consisting of: resiquimod; N-[4-(4-amino-2-ethylimidazo[4,5-c]quinolin-1-yl)butyl]methanesulfonamide); imiquimod; 2-ethoxymethyl)-1H-imidazo[4,5-c]quinolin-4-amine; 2-propylthiazolo[4,5-c]quinolin-4-amine; isatoribine; ANA975, ANA-773; and GS-9620; (y) LIGHT or a nucleic acid encoding LIGHT; (z) antibodies to LAG-3; and (aa) antibodies to CTLA4.

As used herein, unless further defined or limited, the term “antibody” encompasses both polyclonal and monoclonal antibodies, as well as genetically engineered antibodies such as chimeric, humanized or fully human antibodies of the appropriate binding specificity. As used herein, unless further defined, the term “antibody” also encompasses antibody fragments such as sFv, Fv, Fab, Fab′ and F(ab)′₂ fragments. In many cases, it is preferred to use monoclonal antibodies. In some contexts, antibodies can include fusion proteins comprising an antigen-binding site of an antibody, and any other modified immunoglobulin molecule comprising an antigen recognition site (i.e., antigen-binding site) as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins, antineoplastic agents, antimetabolites, or radioisotopes; in some cases, conjugation occurs through a linker or through noncovalent interactions such as an avidin-biotin or streptavidin-biotin linkage.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises an antigen-binding site or epitope-binding site. The term “variable region” of an antibody refers to the variable region of an antibody light chain, or the variable region of an antibody heavy chain, either alone or in combination. The variable regions of the heavy and light chains each consist of four framework regions (FR) connected by three complementarity determining regions (CDRs), also known as “hypervariable regions.” The CDRs in each chain are held together in close proximity by the framework regions and, with the CDRs from the other chain, contribute to the formation of the antigen-binding site of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda, Md.), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs. The term “monoclonal antibody” as used herein refers to a homogeneous antibody population involved in the highly specific recognition and binding of a single antigenic determinant or epitope. This is in contrast to polyclonal antibodies that typically include a mixture of different antibodies directed against a variety of different antigenic determinants. The term “monoclonal antibody” encompasses both intact and full-length monoclonal antibodies as well as antibody fragments (e.g., Fab, Fab′, F(ab′)2, Fv), single chain (sFv) antibodies, fusion proteins comprising an antibody portion, and any other modified immunoglobulin molecule comprising an antigen recognition site (antigen-binding site). Furthermore, “monoclonal antibody” refers to such antibodies made by any number of techniques, including but not limited to, hybridoma production, phage selection, recombinant expression, and expression in transgenic animals. The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability (Jones et al., 1986, Nature, 321:522-525; Riechmann et al., 1988, Nature, 332:323-327; Verhoeyen et al., 1988, Science, 239:1534-1536). In some instances, the Fv framework region residues of a human immunoglobulin are replaced with the corresponding residues in an antibody from a non-human species that has the desired specificity, affinity, and/or binding capability. The humanized antibody can be further modified by the substitution of additional residues either in the Fv framework region and/or within the replaced non-human residues to refine and optimize antibody specificity, affinity, and/or binding capability. In general, the humanized antibody will comprise substantially all of at least one, and typically two or three, variable domains containing all or substantially all of the CDRs that correspond to the non-human immunoglobulin whereas all or substantially all of the framework regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in, for example, U.S. Pat. No. 5,225,539. The term “human antibody” as used herein refers to an antibody produced by a human or an antibody having an amino acid sequence corresponding to an antibody produced by a human. A human antibody may be made using any of the techniques known in the art. This definition of a human antibody specifically excludes a humanized antibody comprising non-human CDRs. The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, or other antibody producing mammal) with the desired specificity, affinity, and/or binding capability, while the constant regions correspond to sequences in antibodies derived from another species (usually human). The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5 or 8-10 amino acids in a unique spatial conformation.

Antibodies that specifically bind to receptors may either inhibit or activate the receptors to which they specifically bind, depending on the specific epitope being targeted by the antibody. In some cases, when antibodies mimic the naturally-occurring agonist for a receptor, the binding of the antibody to the receptor activates the receptor and causes the receptor to initiate signal transduction. In other cases, the antibody may block the binding of the naturally-occurring agonist to the receptor by steric hindrance or other mechanisms.

When nucleic acid molecules, such as nucleic acid molecules encoding a peptide, polypeptide, or protein, are to be delivered in vivo for subsequent expression of the peptide, polypeptide, or protein encoded by the nucleic acid molecule, a vector as known in the art can be used. The terms “vector” or “vectors” refer to a nucleic acid molecule capable of transporting another nucleic acid to which it has been linked. A “vector” as used herein includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors.” A vector as used herein comprises, but is not limited to, a YAC (yeast artificial chromosome), a BAC (bacterial artificial chromosome), a baculovirus vector, a phage, a phagemid, a cosmid, a viral vector, a plasmid, a RNA vector or a linear or circular DNA or RNA molecule which may consist of chromosomal, non chromosomal, semi-synthetic or synthetic DNA. In general, expression vectors of utility in recombinant DNA techniques are often in the form of “plasmids” which refer generally to circular double stranded DNA loops which, in their vector form are not bound to the chromosome. Large numbers of suitable vectors are known to those of skill in the art. Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1 for Saccharomyces cerevisiae; tetracycline, rifampicin or ampicillin resistance in Escherichia coli. Preferably such vectors are expression vectors, wherein a sequence encoding a polypeptide of interest is placed under control of appropriate transcriptional and translational control elements to permit production or synthesis of said polypeptide. Therefore, the polynucleotide is included in an expression cassette. More particularly, the vector comprises a replication origin, a promoter operatively linked to the encoding polynucleotide, a ribosome binding site, a RNA-splicing site (when genomic DNA is used), a polyadenylation site and a transcription termination site. It also can comprise an enhancer or silencer elements. Selection of the promoter will depend upon the cell in which the polypeptide is expressed. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-Pβ-D-thiogalactopyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes. Delivery vectors and vectors can be associated or combined with any cellular permeabilization techniques such as sonoporation or electroporation or derivatives of these techniques. Viral vectors include retrovirus, adenovirus, parvovirus (e.g. adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomega-lovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, and spumavirus. Viral vectors can also include lentiviral vectors, which are HIV-based vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. Such lentiviral vectors can be either integrating or non-integrating. Another alternative is a shuttle vector. A shuttle vector is a vector that can replicate in two different species, such as a prokaryote such as E. coli and in mammalian cells, such as human cells; shuttle vectors that can replicate in both E. coli and in mammalian cells, including human cells, include adenoviral vectors.

When multiple therapeutic agents are administered, each therapeutic agent can be administered separately, or two or more therapeutic agents can be administered in a single pharmaceutical composition. For example, when three therapeutic agents are to be administered, the following possibilities exist. (1) Each of the three therapeutic agents is administered individually; in this case, each agent can be administered in a separate pharmaceutical composition or as the agent alone without use of a pharmaceutical composition for the agent. Further details on the composition and preparation of pharmaceutical compositions are provided below. In this alternative, zero, one, two, or three separate pharmaceutical compositions can be used. (2) Two of the therapeutic agents are administered together in a single pharmaceutical composition, while the third therapeutic agent is administered separately, either as the agent alone or in a separate pharmaceutical composition. (3) All three therapeutic agents are administered together in a single pharmaceutical composition.

As detailed above, another aspect of the present invention is a pharmaceutical composition including eflornithine or a derivative or analog thereof, optionally including another therapeutically active agent or an agent that can potentiate the activity of the eflornithine or a derivative or analog of eflornithine.

In one alternative, a composition according to the present invention can further comprise an agent that increases the ability of the eflornithine or derivative or analog thereof to pass through the blood-brain barrier as described above.

The pharmaceutical composition according to the present invention can, in one alternative, include a prodrug. When a pharmaceutical composition according to the present invention includes a prodrug, prodrugs and active metabolites of a compound may be identified using routine techniques known in the art. See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

When the pharmacologically active compound in a pharmaceutical composition according to the present invention possesses a sufficiently acidic, a sufficiently basic, or both a sufficiently acidic and a sufficiently basic functional group, these group or groups can accordingly react with any of a number of inorganic or organic bases, and inorganic and organic acids, to form a pharmaceutically acceptable salt. Exemplary pharmaceutically acceptable salts include those salts prepared by reaction of the pharmacologically active compound with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, P-hydroxybutyrates, glycolates, tartrates, methane-sulfonates, propanesulfonates, naphthalene-1-sulfonates, naphthalene-2-sulfonates, and mandelates. If the pharmacologically active compound has one or more basic functional groups, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the pharmacologically active compound has one or more acidic functional groups, the desired pharmaceutically acceptable salt may be prepared by any suitable method available in the art, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

Plasma concentrations in the subjects may be between about 60 μM to about 1000 μM. In some embodiments, the plasma concentration may be between about 200 μM to about 800 μM. In other embodiments, the concentration is about 300 μM to about 600 μM. In still other embodiments the plasma concentration may be between about 400 to about 800 μM. In another alternative, the plasma concentration can be between about 0.5 μM to about 20 μM, typically 1 μM to about 10 μM. Suitable plasma concentrations can be determined by those skilled in the art.

The compositions of the invention may be manufactured using techniques generally known for preparing pharmaceutical compositions, e.g., by conventional techniques such as mixing, dissolving, granulating, dragee-making, levitating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations, which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, solutions, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

Pharmaceutical formulations for parenteral administration can include aqueous solutions or suspensions. Suitable lipophilic solvents or vehicles include fatty oils such as sesame oil or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or modulators which increase the solubility or dispersibility of the composition to allow for the preparation of highly concentrated solutions, or can contain suspending or dispersing agents. Pharmaceutical preparations for oral use can be obtained by combining the pharmacologically active agent with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating modulators may be added, such as the cross-linked polyvinyl pyrrolidone, agar, or alginic acid or a salt thereof such as sodium alginate.

Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-a-naphthylamine, or lecithin can be used. Also, chelators such as EDTA can be used. Other ingredients that are conventional in the area of pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, particularly as described above. For administration of any of the compounds used in the present invention, preparations should meet sterility, pyrogenicity, general safety, and purity standards as required by the FDA Office of Biologics Standards or by other regulatory organizations regulating drugs.

For administration intranasally or by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

A pharmaceutical composition can be administered by a variety of methods known in the art. The routes and/or modes of administration vary depending upon the desired results. Depending on the route of administration, the pharmacologically active agent may be coated in a material to protect the therapeutic agent or agents from the action of acids and other compounds that may inactivate the agent. Conventional pharmaceutical practice can be employed to provide suitable formulations or compositions for the administration of such pharmaceutical compositions to subjects. Any appropriate route of administration can be employed, for example, but not limited to, intravenous, parenteral, intraperitoneal, intravenous, transcutaneous, subcutaneous, intramuscular, or oral administration. Depending on the severity of the malignancy or other disease, disorder, or condition to be treated, as well as other conditions affecting the subject to be treated, either systemic or localized delivery of the pharmaceutical composition can be used in the course of treatment. The pharmaceutical composition as described above can be administered together with additional therapeutic agents intended to treat a particular disease or condition, which may be the same disease or condition that the pharmaceutical composition is intended to treat, which may be a related disease or condition, or which even may be an unrelated disease or condition.

As detailed above, eflornithine and derivatives or analogs thereof have been described as effective for the treatment of glioma, in particular with respect to inhibiting or slowing the progression of glioma to a higher grade. However, all forms of cancer are associated with mutation in malignant cells, so eflornithine or derivatives or analogs thereof can be similarly administered to inhibit or slow the advance of other malignancies as well by preventing mutation in the malignant cells. Although eflornithine or its derivatives or analogs can be used to slow the advance of and prevent mutation in many types of cancers, in particular, eflornithine or its derivatives or analogs can be used to treat neuroblastoma. Eflornithine or its derivatives or analogs increase the concentration of p21 (waf1/cip1) and p27kip-1 and this acts as a cause of cell cycle arrest. Among the tumor types for which such observations have been made are leukemia, pancreatic cancer, neuroblastoma, mammary tumors, and gastric cancer.

This is addressed in the following references: (i) P. M. Bauer et al., “Role of p42/p44 Mitogen-Activated-Protein Kinase and p21waf1/cip1 in the Regulation of Vascular Smooth Muscle Cell Proliferation by Nitric Oxide,” Proc. Natl. Acad. Sci. USA 98: 12802-12807 (2001); (ii) S. H. Choi et al., “Polyamine-Depletion Induces p27Kip1 and Enhances Dexamethasone-Induced G1 Arrest and Apoptosis in Human T lymphoblastic Leukemia Cells,” Leuk. Res. 24: 119-127 (2000); (iii) H. Guo et al., “RhoA Stimulates IEC-6 Cell Proliferation by Increasing Polyamine-Dependent Cdk2 Activity,” Am. J. Physiol. Gastrointest. Liver Physiol. 285: G704-713 (2003); (iv) L. Li et al., “JunD Stabilization Results in Inhibition of Normal Intestinal Epithelial Cell Growth through P21 after Polyamine Depletion,” Gastroenterology 123: 764-779 (2002); (v) M. Li et al., “Chemoprevention of Mammary Carcinogenesis in a Transgenic Mouse Model by Alpha-Difluoromethylornithine (DFMO) in the Diet Is associated with Decreased Cyclin D₁ Activity,” Oncoqene 22: 2568-2572 (2003); (vi) A. Mohammed et al., “Eflornithine (DFMO) Prevents Progression of Pancreatic Cancer by Modulating Ornithine Decarboxylase Signaling,” Cancer Prev. Res. 7: 1198-1209 (2014); (vii) T. Nemoto et al., “p53 Independent G(1) Arrest Induced by DL-Alpha-Difluoromethylornithine,” Biochem. Biophys. Res. Commun. 280: 848-854 (2001); (viii) R. M. Ray et al., “Polyamine Depletion Arrests Cell Cycle and Induces Inhibitors p21(Waf1/Cip1), p27(Kip1), and p53 in IEC-6 Cells,” Am. J Physiol. 276: C684-691 (1999); (ix) R. J. Rounbehler et al., “Targeting Ornithine Decarboxylase Impairs Development of MYCN-Amplified Neuroblastoma,” Cancer Res. 69: 547-553 (2009); (x) J. Singh et al., “Modulation of Azoxymethane-Induced Mutational Activation of ras Protooncogenes by Chemopreventive Agents in Colon Carcinogenesis,” Carcinoqenesis 15: 1317-1323 (1994); (xi) R. Singh et al., “Activation of Caspase-3 Activity and Apoptosis in MDA-MB-468 Cells by N(omega)-Hydroxy-L-Arginine, an Inhibitor of Arginase, Is Not Solely Dependent on Reduction in Intracellular Polyamines,” Carcinoqenesis 22: 1863-1869 (2001); (xii) L. Tao et al., “Altered Expression of c-myc, p16 and p27 in Rat Colon Tumors and Its Reversal by Short-Term Treatment with Chemopreventive Agents.” Carcinoqenesis 23: 1447-1454 (2002); (xiii) C. J. Wallick et al., “Key Role for p27Kip1, Retinoblastoma Protein Rb, and MYCN in Polyamine Inhibitor-Induced G1 Cell Cycle Arrest in MYCN-Amplified Human Neuroblastoma Cells,” Oncogene 24: 5606-5618 (2005); (xiv) Q. Xianq et al., “[Apoptotic Induction of Human Lung Carcinoma A549 Cells by DFMO through Fas/FasL Pathway],” Ai Zhenq 12: 1260-1263 (2003); and References (9) and (10), below.

Also, as detailed above, eflornithine or a derivative or analog thereof or a pharmaceutical composition as detailed above including eflornithine or a derivative or analog thereof can be used for the treatment of other malignancies, including, but not limited to, a non-glioma central nervous system malignancy, colorectal cancer, leukemia, lymphoma, pancreatic cancer, liver cancer, and stomach cancer.

The invention is illustrated by the following Examples. These Examples are included for illustrative purposes only, and are not intended to limit the invention.

### Example 1

### Experimental Design to Determine the Effect of Temozolomide and Eflornithine on Mutation Frequency in a Glioblastoma Cell Line Model

A study was performed to explore the effect of temozolomide (TMZ) and eflornithine (DFMO) on mutation frequency of chromosomal abnormalities commonly attributed to primary cancers in a known glioblastoma primary cell line, U87MG.

Frozen U87MG cells were rapidly thawed in a 37° C. water bath, then slowly diluted using pre-warmed growth medium and plated at high density to optimize recovery. The cells were then harvested and placed in T75 flasks with final cell number of 4·10⁶ cells/flask. The flasks were incubated overnight in humidified incubators at 37° C. with 5% CO₂. TMZ solutions in DMSO were prepared at three different effective concentrations: EC-10, EC-20 and EC-50, and applied to the cells at 37.5 μL per flask. Effective concentration (“EC”) was determined in an earlier experiment wherein 50%, 20% and 10% cell survival when exposed to various temozolomide concentrations was defined. The control set of flasks was treated with DMSO at concentration of 0.25% v/v in culture medium. The flasks were incubated for 72 hours before subjecting them to Eflornithine treatments. Eflornithine solutions with 50, 100 and 200 μM concentrations were prepared in culture medium. The eflornithine solutions were applied to the TMZ-treated and untreated cells at 1500 μL per flask and two subsets of cells were incubated for 7 and 14 days, respectively. The cells were then placed into new T75 flasks to obtain cell density of 2 ×10⁶ cells/flask and returned to the incubator for an additional 7 days. After additional incubation, the cells were re-suspended in PBS and centrifuged for 5 minutes at approximately 1000 rpm. The supernatant was removed, and the cell pellet stored at −80° C. before it was used for Exon-Seq analysis. The summary of the treatment groups and testing time points are shown in Table 1.

TABLE 1
Treatment Groups and Testing Time Points

| Treatment | T = 3 days | T = 7 days | T = 14 days |
|---|---|---|---|
| Untreated | X | X | X |
| TMZ EC50 | X | — | — |
| TMZ EC20 | X | — | — |
| TMZ EC10 | X | — | — |
| TMZ EC 50 + DFMO 200 μM* | — | X | X |
| TMZ EC 20 + DFMO 100 μM* | — | X | X |
| TMZ EC 10 + DFMO 50 μM* | — | X | X |

*Cells subjected to respective TMZ treatments for 3 days followed by DFMO treatment for the remaining duration

Exon-Seq analysis was performed per optimized protocol for Illumina paired-end multiplexed library preparation using the SureSelect^{XT} Library Prep and Capture System (Agilent Technologies). DNA was extracted from cells (0.5×10⁶) using commercial DNA kits according to the manufacturer's instructions. For the purposes of library preparation, 300 ng genomic DNA concentrations were measured with the Qubit 2.0 fluorometer dsDNA HS Assay (Thermo Fisher Scientific) and sheared with the Covaris LE220 Sonicator (Covaris) to target 150-200 bp average size. DNA libraries were prepared using the Sureselect^{XT} reagent kit (Agilent Technologies). The 3′ and 5′ overhangs on the DNA fragments were repaired using End Repair Mix (a component of the Sureselect^{XT} kit) and purified using Agencourt AMPure XP Beads (Beckman). The purified fragments were extended with “A” tails using A tailing Mix (component of Sureselect^{XT}) and then ligated with an adapter using DNA ligase (component of Sureselect^{XT}). The adapter-ligated DNA fragments were amplified with Herculase II Fusion DNA Polymerase (Agilent). Finally, the pre-capture libraries containing exome sequences were captured using the SureSelect capture library kit (Agilent). For Illumina sequencing, DNA concentration of the enriched sequencing libraries was measured with the Qubit 2.0 fluorometer dsDNA HS Assay (Thermo Fisher Scientific). Size distribution of the resulting sequencing libraries was analyzed using the Agilent BioAnalyzer 2100 (Agilent). The libraries were used in cluster formation on an Illumina cBOT cluster generation system with HiSeq PE Cluster Kits (Illumina). Paired-end sequencing was performed using an Illumina HiSeq system following Illumina-provided protocols for 2×150 paired-end sequencing. This PCR based amplification method provided a relative number (e.g., fluorescence intensity) for each nucleotide polymorphism detected. This represents the comparative frequency of each mutation across samples (e.g., mutation frequency).

Preliminary data analysis was performed to identify nucleotides susceptible to temozolomide (TMZ)-induced mutations per TMZ concentration. Mutation frequency results were analyzed for 13,040 nucleotide polymorphisms across 6,455 genes that have been previously identified as potentially cancer related based on their description in the Catalogue of Somatic Mutations in Cancer (COSMIC) database. An arbitrary threshold of 15% was used to define what was considered to be a meaningful shift in mutation rate. The results of the preliminary analysis are shown in Table 2.

TABLE 2
Number of Nucleotides Susceptible to TMZ-Induced Mutations

| | Number of nucleotides showing higher |
|---|---|
| | than 15% mutation frequency increase |
| TMZ Concentration | due to TMZ treatment |
| TMZ EC10 | 424 |
| TMZ EC20 | 436 |
| TMZ EC50 | 417 |
| All three concentrations | 97 |
| At least one concentration | 876 |

### Example 2

### Effect of Temozolomide and Eflornithine on Mutation Frequency in Glioblastoma Cell Line Model (Data Analysis for TMZ Concentration EC-10, Eflornithine Concentration 50 μM)

Further analysis of eflornithine effect on mutation frequency was performed for the genes that were identified as susceptible to TMZ-induced mutations in at least one TMZ concentration. Statistical analysis of the Exon-Seq data was performed using JMP™ Software. The mutation frequencies were analyzed using ANOVA analysis of variance and the Student's t-test.

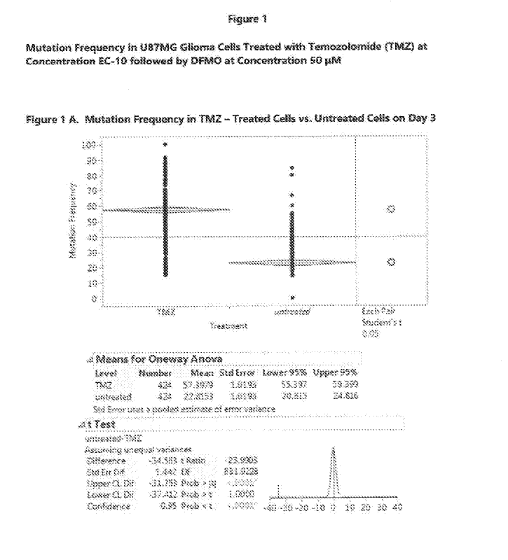

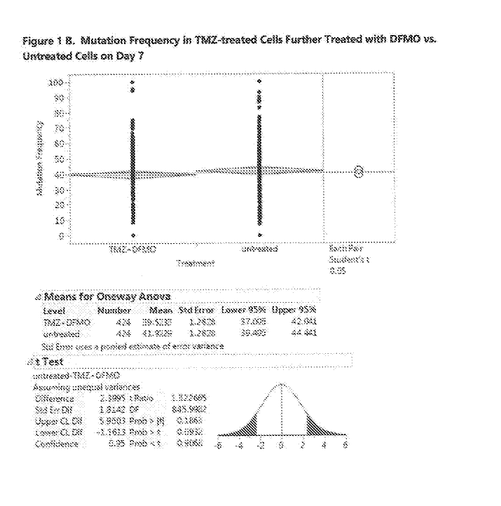

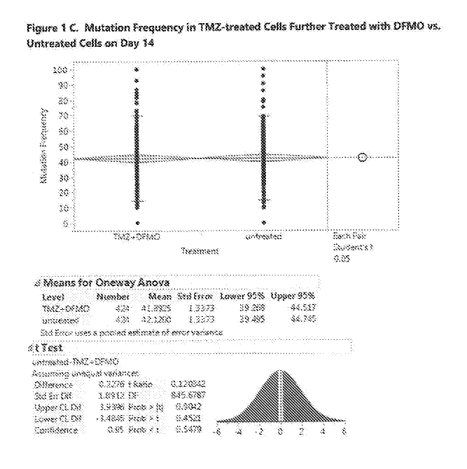

In this Example 2, the mutation frequency was analyzed for the U87MG cells treated with TMZ at EC-10 for three days followed by treatment with eflornithine at concentration of 50 μM for 7 and 14 days. FIG. 1A shows the effect of TMZ on untreated cells on Day 3. The ANOVA analysis and Student's t-test results show that TMZ has a statistically significant effect on cell mutations, raising the mutation frequency on day 3 from the average of 23% for untreated cells to 57% for the cells subjected to TMZ treatment. FIGS. 1B and 1C show the results of mutation frequency measured in cells after subsequent application of DFMO on days 7 and 14, respectively. The results show that subsequent treatment with DFMO brings the average mutation frequency level of TMZ-treated cells to a level statistically similar to the level of mutation in the untreated cells for the respective time point, with the average mutation rate in the range of 40 to 42% for all treatment groups. FIG. 1D summarizes the data for all time points to demonstrate the overall effect of TMZ and subsequent eflornithine treatment on mutation frequency of U87MG cells. Based on the ANOVA analysis and Student's t-test results of the combined data set, TMZ in concentration EC-10 causes significant increase in mutation frequency and sequential eflornithine treatment at a concentration of 50 μM causes a significant decrease in mutation rate compared to the TMZ-treated cells.

### Example 3

### Effect of Temozolomide and Eflornithine on Mutation Frequency in Glioblastoma Cell Line Model (Data Analysis for TMZ Concentration EC-20, Eflornithine Concentration 100 μM)

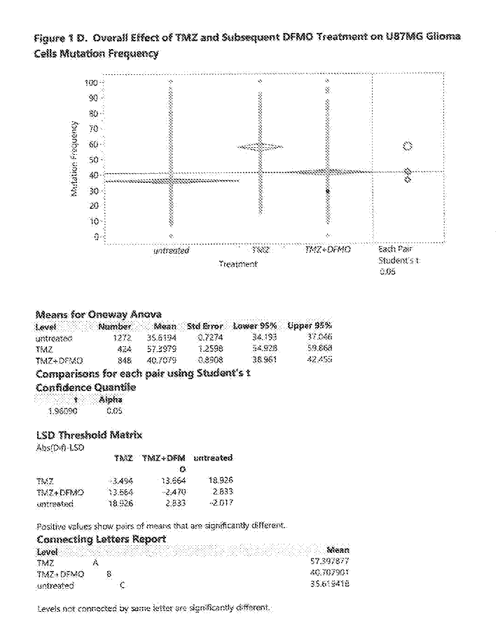

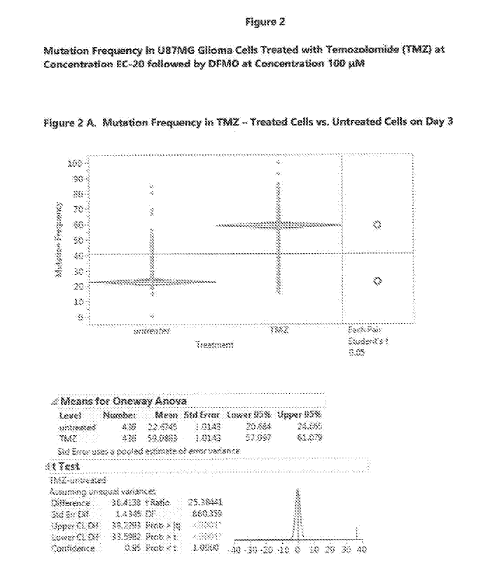

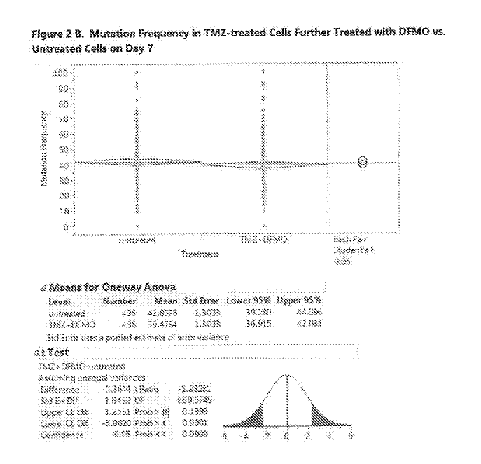

In this Example 3, the mutation frequency was analyzed for the U87MG cells treated with TMZ at EC-20 for three days followed by treatment with eflornithine at concentration of 50 μM for 7 and 14 days. FIG. 2A shows the effect of TMZ on untreated cells on Day 3. The ANOVA analysis and Student's t-test results show that TMZ has a statistically significant effect on cell mutations, raising the mutation frequency on day 3 from the average of 23% for untreated cells to 59% for the cells subjected to TMZ treatment. FIGS. 2B and 2C show the results of mutation frequency measured in cells after subsequent application of eflornithine on days 7 and 14, respectively. The results show that subsequent treatment with eflornithine brings the average mutation frequency level of TMZ-treated cells to a level statistically similar to the level in the untreated cells for the respective time point, with the average mutation rate in the range of 39 to 43% for all treatment groups. FIG. 2D summarizes the data for all time points to demonstrate the overall effect of TMZ and subsequent eflornithine treatment on mutation frequency of U87MG cells. Based on the ANOVA analysis and Student's t-test results of the combined data set, TMZ in a concentration of EC-20 causes a significant increase in mutation frequency and sequential DFMO treatment in a concentration of 100 μM causes a significant decrease in mutation rate compared to the TMZ-treated cells.

### Example 4

### Effect of Temozolomide and Eflornithine on Mutation Frequency in Glioblastoma Cell Line Model (Data Analysis for TMZ Concentration EC-50, Eflornithine Concentration 200 μM)

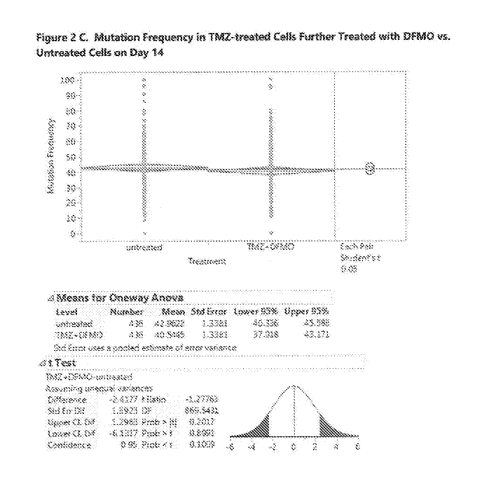

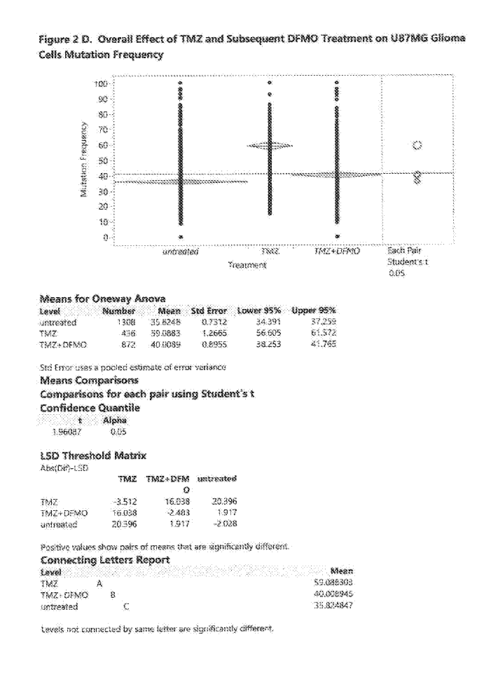

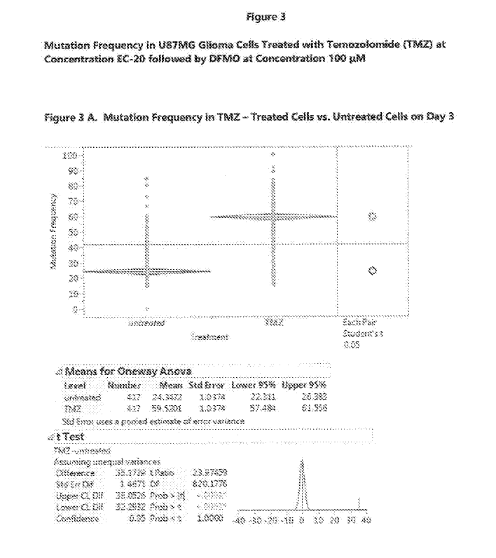

In Example 4, the mutation frequency was analyzed for the U87MG cells treated with TMZ at EC-50 for three days followed by treatment with eflornithine at concentration of 200 μM for 7 and 14 days. FIG. 3A shows the effect of TMZ on untreated cells on Day 3. The ANOVA analysis and Student's t-test results show that TMZ has a statistically significant effect on cell mutations, raising the mutation frequency on day 3 from the average of 24% for untreated cells to 60% for the cells subjected to TMZ treatment. FIGS. 3B and 3C show the results of mutation frequency measured in cells after subsequent application of eflornithine on days 7 and 14, respectively. The results show that subsequent treatment with eflornithine brings the average mutation frequency level of TMZ-treated cells to a level statistically similar to the level in the untreated cells for the respective time point, with the average mutation rate in the range of 40 to 44% for all treatment groups. FIG. 3D summarizes the data for all time points to demonstrate the overall effect of TMZ and subsequent eflornithine treatment on mutation frequency of U87MG cells. Based on the ANOVA analysis and Student's t-test results of the combined data set, TMZ in concentration EC-50 causes a significant increase in mutation frequency and sequential eflornithine treatment in a concentration of 200 μM causes a significant decrease in mutation rate compared to the TMZ-treated cells.

### Example 5

### Effect of Temozolomide and Eflornithine on Mutation Frequency in Glioblastoma Cell Line Model (Summary)

The summary of the results presented in Examples 2-4 is shown in Table 3. Analysis of eflornithine effect on mutation frequency was performed for the genes that were identified as susceptible to TMZ-induced mutations in at least one TMZ concentration.

TABLE 3
Mean Mutation Frequency in Glioblastoma Cell Line Model

| | Mutation Frequency | | |
|---|---|---|---|
| Treatment | T = 3 days | T = 7 days | T = 14 days |
| Untreated (averages) | 23% | 42% | 43% |
| TMZ EC10 | 57% | — | — |
| TMZ EC20 | 59% | — | — |
| TMZ EC50 | 60% | — | — |
| TMZ EC 10 + DFMO 50 μM* | — | 40% | 42% |
| TMZ EC 20 + DFMO 100 μM* | — | 39% | 41% |
| TMZ EC 50 + DFMO 200 μM* | — | 40% | 42% |

This data confirms that mutation frequency in genes susceptible to TMZ-induced mutations can be lowered by sequential eflornithine treatment.

### Example 6

### Effect of Temozolomide and Eflornithine on Mutation Frequency in Specific Gene of Glioblastoma Cell Line Model (TP53BP1)

In this Example 6, the frequency of mutation of a specific gene known to play an important role in carcinogenesis is reviewed in conjunction with TMZ and eflornithine treatments.

TP53BP1, also known as “TP53,” is a gene located on chromosome 15, whose activity has been described above. TP53 has been linked to mutations present in post-temozolomide treated gliomas.

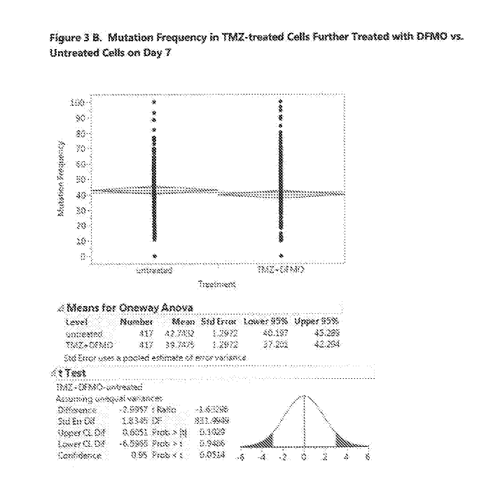

In this experiment, conducted as described in Example 1, the U87MG cells showed C/T mutation in TP53BP1 gene location chr15: 43,762,196. The frequency of this mutation in the untreated cells on day 3 was 14.8%. Comparatively, the cells subjected to TMZ treatment at EC-50 concentration showed the mutation frequency of 62.5% on day 3. As the experiment continued, the untreated cells reached mutation frequency of 53.1% on day 7 and 48.4% on day 14. The cells which were treated with TMZ for 3 days and then subjected to eflornithine treatment at 200 μM concentration showed the reduced mutation rates of 29.7% and 38.4% at 7 and 14 days, respectively. The results of this experiment are illustrated in FIG. 4.

### Example 7

### Effect of Temozolomide and Eflornithine on Mutation Frequency in Specific Gene of Glioblastoma Cell Line Model (ADAM32)

In this Example 7, the frequency of mutation of a specific gene known to play an important role in carcinogenesis is reviewed in conjunction with TMZ and eflornithine treatments.

ADAM32, located on chromosome 8, also known as A Disintegrin And Metalloproteinase Domain 32, is a gene encoding a member of the disintegrin family of membrane anchored proteins. Mutations in this gene have been associated with hypermutation in gliomas.

In this experiment, conducted as described in Example 1, the U87MG cells showed G/A mutation in the ADAM 32 gene location chr8: 38,964,647. The frequency of this mutation in the untreated cells on day 3 was 48.1%. The cells subjected to TMZ treatment at EC-50 concentration showed the mutation frequency of 63.6% on day 3. As the experiment continued, the untreated cells mutation remained at similar rate of 50.0% on day 7 and 45.1% on day 14. The cells which were treated with TMZ for 3 days and then subjected to eflornithine treatment at 200 μM concentration showed the reduced mutation rates of 33.3% and 43.2% at 7 and 14 days, respectively.

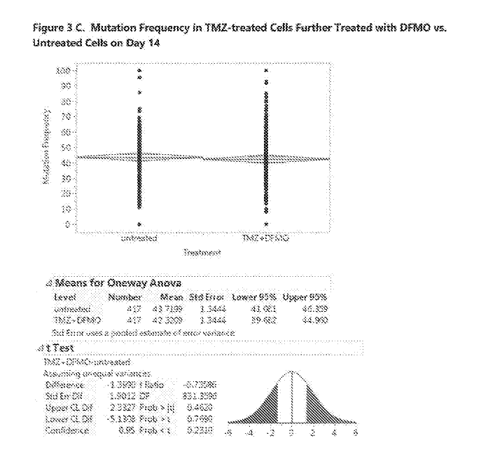

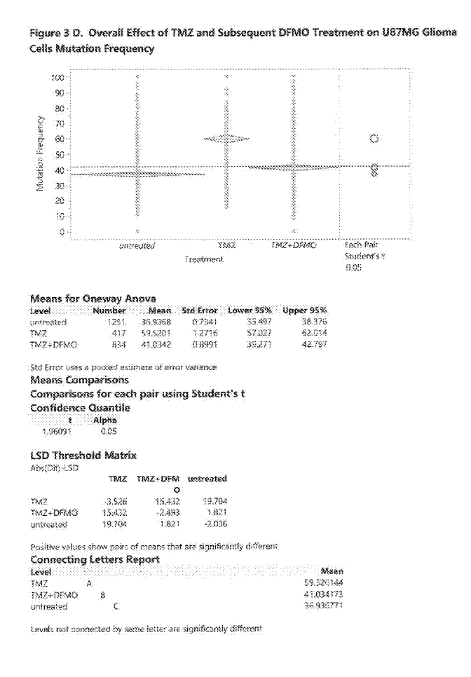

The results of this experiment are illustrated in FIG. 5.

### Example 8

### Effect of Temozolomide and Eflornithine on Mutation Frequency in Specific Gene of Glioblastoma Cell Line Model (GPR116)

In Example 8, the frequency of mutation of a specific gene known to play an important role in carcinogenesis is reviewed in conjunction with TMZ and eflornithine treatments.

GPR116, also known as ADGRF5, is located on chromosome 6 and encodes a G protein-coupled receptor (GPR), G protein-coupled receptor 116. GPR116 has been identified as a hypermutating gene in gliomas after temozolomide treatment. GPRs are involved in cellular proliferation and evading apoptosis.

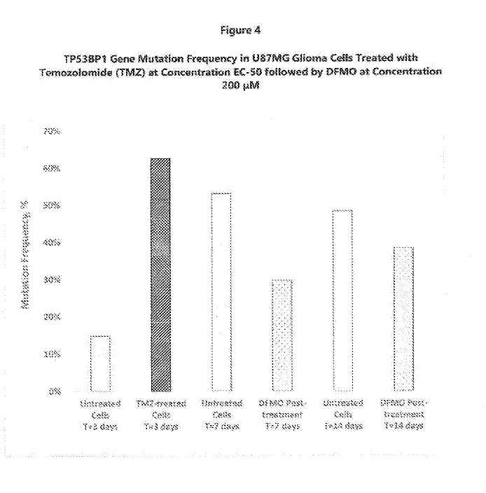

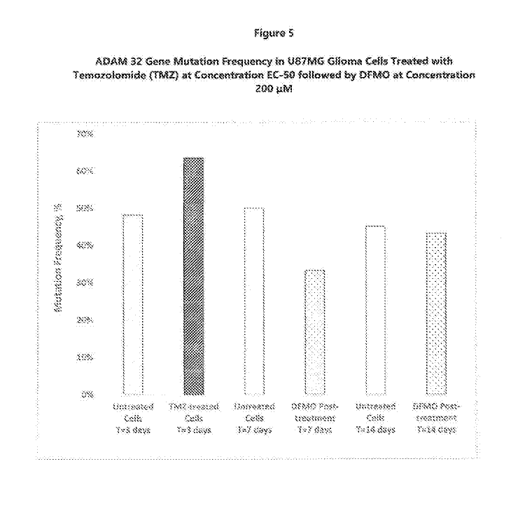

In this experiment, conducted as described in Example 1, the U87MG cells showed C/T mutation in GPR116 gene location chr6: 46,867,771. The frequency of this mutation in the untreated cells on day 3 was 46.0%. The cells subjected to TMZ treatment at EC-20 concentration showed the mutation frequency of 69.6% on day 3. As the experiment continued, the untreated cells mutation reached 48.4% on day 7 and 52.4% on day 14. The cells which were treated with TMZ for 3 days and then subjected to eflornithine treatment at 100 μM concentration showed the mutation rates of 60.6% and 45.0% at 7 and 14 days, respectively. The results of this experiment are illustrated in FIG. 6.

### Example 9

### Effect of Temozolomide and Eflornithine on Mutation Frequency in Specific Gene of Glioblastoma Cell Line Model (MUC16)

In Example 9, the frequency of mutation of a specific gene known to play an important role in carcinogenesis is reviewed in conjunction with TMZ and eflornithine treatments.

MUC16, located on chromosome 19, encodes a protein that is a member of the mucin family. MUC16 is involved in cell migration and is implicated in mutations present in post-temozolomide treated gliomas.

In this experiment, conducted as described in Example 1, the U87MG cells showed a T/A mutation in MUC16 gene location chr19: 9,071,763. The frequency of this mutation in the untreated cells on day 3 was 43.0%. The cells subjected to TMZ treatment at EC-50 concentration showed the mutation frequency of 64.0% on day 3.

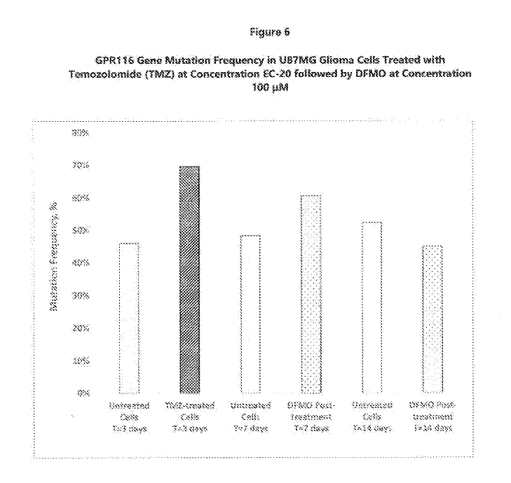

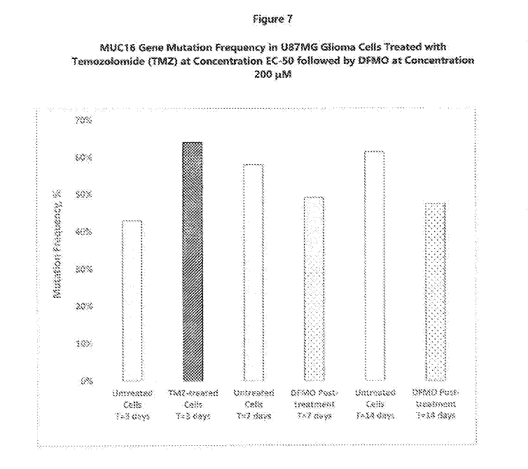

As the experiment continued, the untreated cells mutation rate reached 58.0% on day 7 and 61.3% on day 14. The cells which were treated with TMZ for 3 days and then subjected to eflornithine treatment at 200 μM concentration showed the mutation rates of 49.1% and 47.3% at 7 and 14 days, respectively. The results of this experiment are illustrated in FIG. 7.

The following publications are incorporated herein by this reference. These publications are referred to herein by the numbers provided below. The inclusion of any publication in this list of publications is not to be taken as an admission that any publication referred to herein is prior art.
1. Metcalf R, Bey P, Danzin C, Jung M J, Casara P, Vevert J P. Catalytic irreversible inhibition of mammalian ornithine decarboxylase (EC 4.1.1.17) by substrate and analog product analogs. J Am Chem Soc. 1978; 100:2551-2552.
2. Bacchi C J, Garofalo J, Mockenhaupt D, et al. In vivo effects of alpha-D L-difluoromethylornithine on the metabolism and morphology of Trypanosoma brucei brucei. Mol Biochem Parasitol. March 1983; 7(3):209-225.
3. Bacchi C J, Nathan H C, Hutner S H, McCann P P, Sjoerdsma A. Polyamine metabolism: a potential therapeutic target in trypanosomes. Science. Oct. 17 1980; 210(4467):332-334.
4. Shantz L M, Levin V A. Regulation of ornithine decarboxylase during oncogenic transformation: mechanisms and therapeutic potential. Amino Acids. August 2007; 33(2):213-223.
5. Childs A C, Mehta D J, Gerner E W. Polyamine-dependent gene expression. Cell Mol Life Sci. July 2003; 60(7):1394-1406.
6. Gerner E W, Meyskens F L, Jr. Polyamines and cancer: old molecules, new understanding. Nat Rev Cancer. October 2004; 4(10):781-792.
7. Levin V A, Hess K R, Choucair A, et al. Phase III randomized study of postradiotherapy chemotherapy with combination alpha-difluoromethylornithine-PCV versus PCV for anaplastic gliomas. Clinical Cancer Research. March 2003; 9(3):981-990.
8. Levin V A, Hess K R, Choucair A K, et al. Final report for evaluable patients treated on DM92-035, phase III randomized study of post-irradiation PCV versus DFMO-PCV, for anaplastic gliomas (AG). Neuro Oncol. 2012; 14(Supplement 6):vi74.
9. Koomoa D L, Yco L P, Borsics T, Wallick C J, Bachmann A S. Ornithine decarboxylase inhibition by DFMO activates opposing signaling pathways via phosphorylation of both Akt/PKB and p27Kip1 in neuroblastoma. Cancer Res. Dec. 1, 2008; 68(23):9825-9831.
10. Koomoa D L, Geerts D, Lange I, et al. DFMO/eflornithine inhibits migration and invasion downstream of MYCN and involves p27Kip1 activity in neuroblastoma. Int J Oncol. April 2013; 42(4): 1219-1228.
11. Johnson B E, Mazor T, Hong C, et al. Mutational Analysis Reveals the Origin and Therapy-Driven Evolution of Recurrent Glioma. Science. Dec. 12, 2013.
12. Hunter C, Smith R, Cahill D P, et al. A hypermutation phenotype and somatic MSH6 mutations in recurrent human malignant gliomas after alkylator chemotherapy. Cancer Res. Apr. 15, 2006; 66(8):3987-3991.
13. Yip S, Miao J, Cahill D P, et al. MSH6 mutations arise in glioblastomas during temozolomide therapy and mediate temozolomide resistance. Clin Cancer Res. Jul. 15, 2009; 15(14):4622-4629.
14. The Cancer Genome Atlas Research Network. Comprehensive genomic characterization defines human glioblastoma genes and core pathways. Nature. 2008; 455:1061-1068.
15. Bodell W J, Gaikwad N W, Miller D, Berger M S. Formation of DNA adducts and induction of lacI mutations in Big Blue Rat-2 cells treated with temozolomide: implications for the treatment of low-grade adult and pediatric brain tumors. Cancer Epidemiol Biomarkers Prev. June 2003; 12(6):545-551.
16. Einspahr J G, Nelson M A, Saboda K, Warneke J, Bowden G T, Alberts D S. Modulation of biologic endpoints by topical difluoromethylornithine (DFMO), in subjects at high-risk for nonmelanoma skin cancer. Clin Cancer Res. January 2002; 8(1): 149-155.
17. Hoshino T, Prados M, Wilson C B, Cho K G, Lee K S, Davis R L. Prognostic implications of the bromodeoxyuridine labeling index of human gliomas. J Neurosurg. 1989; 71(3):335-341.
18. Labrousse F, Daumas-Duport C, Batorski L, Hoshino T. Histological grading and bromodeoxyuridine labeling index of astrocytomas. Comparative study in a series of 60 cases. J Neurosurg. 1991; 75(2):202-205.
19. Prados M D, Krouwer H G, Edwards M S, Cogen P H, Davis R L, Hoshino T. PROLIFERATIVE POTENTIAL AND OUTCOME IN PEDIATRIC ASTROCYTIC TUMORS. J Neurooncol. 1992; 13(3):277-282.
20. Hoshino T, Ahn D, Prados M D, Lamborn K, Wilson C B. Prognostic significance of the proliferative potential of intracranial gliomas measured by bromodeoxyuridine labeling. Int J Cancer. 1993 1993; 53(4):550-555.
21. Ito S, Chandler K L, Prados M D, et al. Proliferative potential and prognostic evaluation of low-grade astrocytomas. J Neuro-Oncol. 1994 1994; 19(1):1-9.
22. Onda K, Davis R L, Shibuya M, Wilson C B, Hoshino T. Correlation between the bromodeoxyuridine labeling index and the MIB-1 and Ki-67 proliferating cell indices in cerebral gliomas. Cancer. 1994 1994; 74(7):1921-1926.
23. Kajiwara Y, Panchabhai S, Levin V A. A new preclinical 3-dimensional agarose colony formation assay. Technol Cancer Res Treat. August 2008; 7(4):329-334.
24. Kajiwara Y, Panchabhai S, Liu D D, Kong M, Lee J J, Levin V A. Melding a New 3-Dimensional Agarose Colony Assay with the E(max) Model to Determine the Effects of Drug Combinations on Cancer Cells. Technol Cancer Res Treat. April 2009; 8(2): 163-176.
25. Levin V A, Panchabhai S C, Shen L, Kornblau S M, Qiu Y, Baggerly K A. Different changes in protein and phosphoprotein levels result from serum starvation of high-grade glioma and adenocarcinoma cell lines. J Proteome Res. January 2010; 9(1): 179-191.
26. Levin V A, Panchabhai S, Shen L, Baggerly K A. Protein and phosphoprotein levels in glioma and adenocarcinoma cell lines grown in normoxia and hypoxia in monolayer and three-dimensional cultures. Proteome Sci. Jan. 25, 2012; 10(1):5.

### Advantages of the Invention

The present invention, including the methods and compositions described herein, provides a novel and effective method for the treatment of glioma, in particular gliomas of low grade (WHO Grade II) and mid-grade (WHO Grade III), and can prevent progression of these gliomas to a higher grade. Methods and compositions of the present invention can protect against progression of anaplastic gliomas (especially anaplastic astrocytoma) to a more malignant phenotype, such as glioblastoma. These methods and compositions are well-tolerated, do not produce significant side effects, and can be used together with other anti-neoplastic agents, including conventionally used anti-neoplastic agents and immunomodulatory treatments and agents. These methods and compositions as described herein can also be used to treat malignancies other than glioma, including, but not limited to, a non-glioma central nervous system malignancy, colorectal cancer, leukemia, lymphoma, pancreatic cancer, liver cancer, and stomach cancer.

Methods according to the present invention possess industrial applicability for the preparation of a medicament for the treatment of glioma. Compositions according to the present invention possess industrial applicability as pharmaceutical compositions, particularly for the treatment of glioma as well as other malignancies.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. As used herein, the transitional phrase “comprising” also encompasses the transitional phrases “consisting essentially of” and “consisting of” unless either or both of the narrower transitional phrases “consisting essentially of” and “consisting of” are expressly excluded. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

What is claimed is:

1. A method for the treatment of glioma comprising the step of administering a therapeutically effective quantity of eflornithine or a derivative or analog thereof to a subject with glioma in order to reduce the rate of hypermutation of the glioma to reduce the progression or grade of malignancy of the glioma caused by alkylating therapy exposure.

2. The method of claim 1 wherein a gene undergoing hypermutation is at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16.

3. The method of claim 2 wherein a gene undergoing hypermutation is at least one gene selected from the group consisting of H1FNT, RFX1, DHX36, MBD2, FRK, PEG3, NDC80, ABCA6, DNLZ, MUC12, PDCD6IP, and GSC2.

4. The method of claim 1 wherein the subject with glioma is currently treated with an alkylating agent.

5. The method of claim 3 wherein the alkylating agent is selected from the group consisting of temozolomide and lomustine.

6. The method of claim 3 wherein the alkylating agent is selected from the group consisting of:
(i) cyclophosphamide;
(ii) mechlorethamine;
(iii) uracil mustard;
(iv) melphalan;
(v) chlorambucil;
(vi) ifosfamide;
(vii) bendamustine;
(viii) carmustine;
(ix) streptozotocin;
(x) busulfan;
(xi) procarbazine;
(xii) dacarbazine;
(xiii) mitocarbazine;
(xiv) altretamine;
(xv) 6-methyluracil mustard;
(xvi) 6-ethyluracil mustard;
(xvii) 6-propyluracil mustard;
(xviii) 3-[1-methyl-4-[1-methyl-4-[1-methyl-4-[N1-[5-bis(2-chloroethyl)amino-2,4-(1H,3H)pyrimidinedione]acetylamino]-pyrrole-2-carboxamido]pyrrole-2-carboxamido]pyrrole-2-carboxamido]propionamidine hydrochloride;
(xix) 3-[1-methyl-4-[1-methyl-4-[1-methyl-4-[N1-[5-bis(2-chloroethyl)amino-2,4-(1H,3H)pyrimidinedione]propanoylamino]-pyrrole-2-carboxamido]pyrrole-2-carboxamido]pyrrole-2-carboxamido]propionamidine hydrochloride;
(xx) 3-[1-methyl-4-[1-methyl-4-[1-methyl-4-[N1-[5-bis(2-chloroethyl)amino-2,4-(1H,3H)pyrimidinedione]butanoylamino]-pyrrole-2-carboxamido]pyrrole-2-carboxamido]pyrrole-2-carboxamido]propionamidine hydrochloride;
(xxi) 3-[1-methyl-4-[1-methyl-4-[1-methyl-4-[N1-[5-bis(2-chloroethyl)amino-2,4-(1H,3H)pyrimidinedione]pentanoylamino]-pyrrole-2-carboxamido]pyrrole-2-carboxamido]pyrrole-2-carboxamido]propionamidine hydrochloride;
(xxii) 3-[1-methyl-4-[1-methyl-4-[1-methyl-4-[N1-[5-bis(2-chloroethyl)amino-2,4-(1H,3H)pyrimidinedione]hexanoylamino]-pyrrole-2-carboxamido]propionamidine hydrochloride;
(xxiii) 3-[1-methyl-4-[1-methyl-4-[1-methyl-4-[N1-[5-bis(2-chloroethyl)amino-2,4-(1H,3H)pyrimidinedione]heptanoylamino]-pyrrole-2-carboxamido]propionamidine hydrochloride;
(xxiv) estramustine;
(xxv) quinacrine mustard dihydrochloride;
(xxvi) spiromustine;
(xxvii) mustamine;
(xxviii) phenylalanine mustard;
(xxix) mannomustine;
(xxx) 5-((bis(2-chloroethyl)amino)methyl)-pyrimidine-2,4(1H,3H)-dione;
(xxxi) 5-((bis(2-chloroethyl)amino)methyl)-6-methylpyrimidine-2,4(1H,3H)-dione;
(xxxii) 5-((bis(2-chloroethyl)amino)methyl)-1-methylpyrimidine-2,4(1H,3H)-dione;
(xxxiii) 5-((bis(2-chloroethyl)amino)methyl)-1,3-dimethylpyrimidine-2,4(1H,3H)-dione;
(xxxiv) 5-((bis(2-chloroethyl)amino)methyl)-6-propylpyrimidine-2,4(1H,3H)-dione;
(xxxv) 5-((bis(2-chloroethyl)amino)methyl)-6-methyl-2-thioxo-2,3-dihydropyrimidin-4(1H)-one;
(xxxvi) nitrouracil;
(xxxvii) 5,6-dihydro-5-nitrouracil;
(xxxviii) 5,6-dihydro-5-nitro-1-(4-nitrophenyl)uracil;
(xxxix) 5-nitro-1-(4-nitrophenyl)uracil;
(xl) 5,6-dihydro-5-nitro-1 (β-D-ribofuranuronic acid ethyl ester)uracil;
(xli) 5-nitro-1 (β-D-ribofuranuronic acid ethyl ester)uracil;
(xlii) 5-nitrouracil N-oxide;
(xliii) prednimustine;
(xliv) nimustine;
(xlv) ranimustine;
(xlvi) fotemustine;
(xlvii) ribomustine;
(xlviii) cystemustine;
(xlix) 4-chlorouracil mustard;
(l) 4-cyanouracil mustard;
(li) 4-nitrouracil mustard;
(lii) dianhydrogalactitol;
(liii) diacetyldianhydrogalactitol; and
(liv) dibromodulcitol.

7. The method of claim 1 wherein the subject with glioma was previously treated with an alkylating agent.

8. The method of claim 2 further comprising the step of detecting the mutation.

9. The method of claim 8 wherein the mutation is detected by DNA sequencing.

10. The method of claim 1 wherein the eflornithine or derivative or analog thereof is selected from the group consisting of eflornithine and a pharmaceutically acceptable salt form, hydrate, or solvate thereof.

11. The method of claim 10 wherein the eflornithine is selected from the group consisting of: (i) a racemic mixture of D-eflornithine and L-eflornithine; (ii) D-eflornithine; and (iii) L-eflornithine.

12. The method of claim 1 wherein the eflornithine or derivative or analog thereof is a derivative or analog of eflornithine selected from the group consisting of:
(a) an analog of eflornithine of Formula (III): 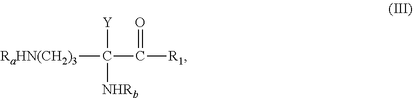
wherein:
(1) Y is FCH₂—, F₂CH—, or F₃C—;
(2) Rₐ and R_{b} are, independently, hydrogen, (C₁-C₄) alkylcarbonyl, or a group of Formula (III(a)) 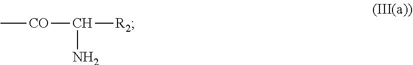
wherein, in Formula (III(a)), R₂ is hydrogen, (C₁-C₄) alkyl, benzyl, or p-hydroxybenzyl;
(3) R₁ is hydroxyl, (C₁-C₈) alkoxy, —NR₄R₅, wherein R₄ and R₅ are independently hydrogen, (C₁-C₄) alkyl, or a group of Formula (III(b)) 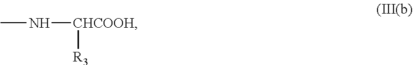
wherein, in Formula (III(b), R₃ is hydrogen, C₁-C₄) alkyl, or p-hydroxybenzyl;
(b) an analog of eflornithine of Formula (IV) or (V): 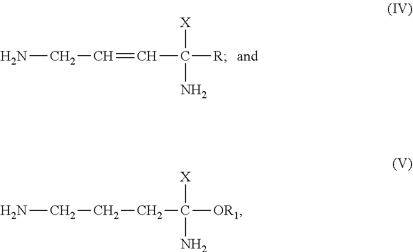
wherein:
(1) X is —CHF₂ or —CH₂F;
(2) R is hydrogen or —COR₁; and
(3) R₁ is —OH or (C₁-C₆) alkoxy;
(c) a water-soluble salt of eflornithine with a polycation selected from the group consisting of a polycationic carbohydrate, a polyaminoacid, a polyamine, a polypeptide, a basic polymer, or a quaternary ammonium compound;
(d) a conjugate in which a first moiety that is eflornithine or a derivative or analog of eflornithine is covalently linked to a second moiety that is a non-steroidal anti-inflammatory drug; and
(e) a copolymer of formula A-B-C or a pharmaceutically acceptable salt thereof, wherein A comprises a water soluble polymer; B comprises a matrix metalloprotease (MMP)-cleavable polypeptide; C is eflornithine; and A is connected to B at a first end through a first covalent bond or a first linking moiety and B is connected to C at a second end through a second covalent bond or a second linking moiety, and wherein the co-polymer is not crosslinked.

13. The method of claim 1 wherein the eflornithine or derivative or analog thereof is administered orally or by injection.

14. The method of claim 1 wherein the eflornithine or derivative or analog thereof is administered together with or adjuvant to radiotherapy.

15. The method of claim 1 wherein the glioma is characterized by one or more of the following characteristics:
(a) the glioma was previously treated with radiation therapy and adjuvant alkylator therapy and is recurrent/refractory anaplastic glioma;
(b) the glioma has a mutation in one or more genes selected from the group consisting of IDH1, IDH2, TP53, PTEN, ATRX, BRAF, CDKEN2A, SMARCA4, and PIK3;
(c) the glioma has the promoter for MGMT methylated; and
(d) the glioma has a mutation in at least one other gene that affects proliferation, survival, or resistance to chemotherapy.

16. The method of claim 1 wherein the eflornithine or derivative or analog thereof is administered together with a therapeutically effective quantity of one or more conventional antineoplastic agents used for the treatment of glioma, wherein the one or more conventional antineoplastic agents are selected from the group consisting of: alkylating agents, platinum-containing agents that damage DNA, antimetabolites, anti-angiogenic agents, EGFR inhibitors, topoisomerase inhibitors, and tyrosine kinase inhibitors.

17. The method of claim 1 wherein the eflornithine or derivative or analog thereof is administered together with an additional agent selected from the group consisting of:
(a) an inhibitor of polyamine transport;
(b) a polyamine analog;
(c) an S-adenosylmethionine decarboxylase inhibitor;
(d) an agent selected from the group consisting of: (1) a retinoid; (2) a syrbactin compound; (3) a cyclooxygenase-2 inhibitor; (4) a non-steroidal anti-inflammatory agent; (5) castanospermine or castanospermine esters; (6) an aziridinyl putrescine compound; (7) an interferon; (8) an aryl substituted xylopyranoside derivative; (9) an agent that reduces blood glutamate levels and enhances brain to blood glutamate efflux; (10) chitosan or chitosan derivatives and analogs; (11) 2,4-disulfonyl phenyl tert-butyl nitrone; (12) 3-(4-amino-1-oxo-1,3-dihydro-isoindol-2-yl)-piperidine-2,6-dione; (13) thalidomide; (14) N-2-pyridinyl-2-pyridinecarbothioamide; (15) cambendazole; and (16) an inhibitor of histone demethylase; and
(e) an agent that increases the ability of the eflornithine or derivative or analog thereof to pass through the blood-brain barrier.

18. The method of claim 1 wherein the eflornithine or derivative or analog of eflornithine is administered in a quantity sufficient to modulate an immune response to the glioma.

19. The method of claim 1 wherein the eflornithine or derivative or analog thereof is administered together with a therapeutically effective quantity of one or more immunomodulatory agents used for the treatment of glioma.

20. The method of claim 18 wherein the immunomodulatory agent is selected from the group consisting of:
(a) IL-15;
(b) anti-PD1 antibodies;
(c) anti-B7-H1 antibodies;
(d) IL-12;
(e) QS-21;
(f) CD-40;
(g) anti-CD40 antibody acting as a CD40 agonist;
(h) CD40L;
(i) IL-7;
(j) CpG;
(k) 1-methyltryptophan;
(l) anti-CD137 antibodies;
(m) anti-TGF-0 antibodies;
(n) anti-IL10 antibodies;
(o) anti-ILR10R antibodies;
(p) Flt3L;
(q) Anti-GITR;
(r) CCL21 or a nucleic acid encoding CCL21;
(s) monophosphoryl lipid A;
(t) poly I:C;
(u) poly ICLC;
(v) anti-OX40 antibodies;
(w) anti-B7-H4 antibodies;
(x) an immune response modulator selected from the group consisting of: resiquimod; N-[4-(4-amino-2-ethylimidazo[4,5-c]quinolin-1-yl)butyl]methanesulfonamide); imiquimod; 2-ethoxymethyl)-1H-imidazo[4,5-c]quinolin-4-amine; 2-propylthiazolo[4,5-c]quinolin-4-amine; isatoribine; ANA975, ANA-773; and GS-9620;
(y) LIGHT or a nucleic acid encoding LIGHT;
(z) antibodies to LAG-3; and
(aa) antibodies to CTLA4.

21. A method for the treatment of a malignancy selected from the group consisting of a non-glioma central nervous system malignancy, colorectal cancer, leukemia, lymphoma, pancreatic cancer, liver cancer, and stomach cancer comprising the step of administering a therapeutically effective quantity of eflornithine or a derivative or analog thereof to a subject with the malignancy in order to reduce the rate of hypermutation of the malignancy to reduce the progression of the malignancy caused by alkylating therapy exposure.

22. The method of claim 21 wherein the reduction of the rate of hypermutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16.

23. The method of claim 22 wherein the mutation of at least one gene is selected from the group consisting of H1FNT, RFX1, DHX36, MBD2, FRK, PEG3, NDC80, ABCA6, DNLZ, MUC12, PDCD6IP, and GSC2.

24. The method of claim 21 wherein the subject with the malignancy is currently treated with an alkylating agent.

25. The method of claim 24 wherein the alkylating agent is selected from the group consisting of temozolomide and lomustine.

26. The method of claim 24 wherein the alkylating agent is selected from the group consisting of:
(i) cyclophosphamide;
(ii) mechlorethamine;
(iii) uracil mustard;
(iv) melphalan;
(v) chlorambucil;
(vi) ifosfamide;
(vii) bendamustine;
(viii) carmustine;
(ix) streptozotocin;
(x) busulfan;
(xi) procarbazine;
(xii) dacarbazine;
(xiii) mitocarbazine;
(xiv) altretamine;
(xv) 6-methyluracil mustard;
(xvi) 6-ethyluracil mustard;
(xvii) 6-propyluracil mustard;
(xviii) 3-[1-methyl-4-[1-methyl-4-[1-methyl-4-[N1-[5-bis(2-chloroethyl)amino-2,4-(1H,3H)pyrimidinedione]acetylamino]-pyrrole-2-carboxamido]propionamidine hydrochloride;
(xix) 3-[1-methyl-4-[1-methyl-4-[1-methyl-4-[N1-[5-bis(2-chloroethyl)amino-2,4-(1H,3H)pyrimidinedione]propanoylamino]-pyrrole-2-carboxamido]propionamidine hydrochloride;
(xx) 3-[1-methyl-4-[1-methyl-4-[1-methyl-4-[N1-[5-bis(2-chloroethyl)amino-2,4-(1H,3H)pyrimidinedione]butanoylamino]-pyrrole-2-carboxamido]propionamidine hydrochloride;
(xxi) 3-[1-methyl-4-[1-methyl-4-[1-methyl-4-[N1-[5-bis(2-chloroethyl)amino-2,4-(1H,3H)pyrimidinedione]pentanoylamino]-pyrrole-2-carboxamido]propionamidine hydrochloride;
(xxii) 3-[1-methyl-4-[1-methyl-4-[1-methyl-4-[N1-[5-bis(2-chloroethyl)amino-2,4-(1H,3H)pyrimidinedione]hexanoylamino]-pyrrole-2-carboxamido]propionamidine hydrochloride;
(xxiii) 3-[1-methyl-4-[1-methyl-4-[1-methyl-4-[N1-[5-bis(2-chloroethyl)amino-2,4-(1H,3H)pyrimidinedione]heptanoylamino]-pyrrole-2-carboxamido]propionamidine hydrochloride;
(xxiv) estramustine;
(xxv) quinacrine mustard dihydrochloride;
(xxvi) spiromustine;
(xxvii) mustamine;
(xxviii) phenylalanine mustard;
(xxix) mannomustine;
(xxx) 5-((bis(2-chloroethyl)amino)methyl)-pyrimidine-2,4(1H,3H)-dione;
(xxxi) 5-((bis(2-chloroethyl)amino)methyl)-6-methylpyrimidine-2,4(1H,3H)-dione;
(xxxii) 5-((bis(2-chloroethyl)amino)methyl)-1-methylpyrimidine-2,4(1H,3H)-dione;
(xxxiii) 5-((bis(2-chloroethyl)amino)methyl)-1,3-dimethylpyrimidine-2,4(1H,3H)-dione;
(xxxiv) 5-((bis(2-chloroethyl)amino)methyl)-6-propylpyrimidine-2,4(1H, 3H)-dione;
(xxxv) 5-((bis(2-chloroethyl)amino)methyl)-6-methyl-2-thioxo-2,3-dihydropyrimidin-4(1H)-one;
(xxxvi) nitrouracil;
(xxxvii) 5,6-dihydro-5-nitrouracil;
(xxxviii) 5,6-dihydro-5-nitro-1-(4-nitrophenyl)uracil;
(xxxix) 5-nitro-1-(4-nitrophenyl)uracil;
(xl) 5,6-dihydro-5-nitro-1(β-D-ribofuranuronic acid ethyl ester)uracil;
(xli) 5-nitro-1(β-D-ribofuranuronic acid ethyl ester)uracil;
(xlii) 5-nitrouracil N-oxide;
(xliii) prednimustine;
(xliv) nimustine;
(xlv) ranimustine;
(xlvi) fotemustine;
(xlvii) ribomustine;
(xlviii) cystemustine;
(xlix) 4-chlorouracil mustard;
(l) 4-cyanouracil mustard;
(li) 4-nitrouracil mustard;
(lii) dianhydrogalactitol;
(liii) diacetyldianhydrogalactitol; and
(liv) dibromodulcitol.

27. The method of claim 21 wherein the subject with the malignancy was previously treated with an alkylating agent.

28. The method of claim 21 further comprising the step of detecting the mutation.

29. The method of claim 28 wherein the mutation is detected by DNA sequencing.

30. The method of claim 21 wherein the eflornithine or derivative or analog thereof is selected from the group consisting of eflornithine and a pharmaceutically acceptable salt form, hydrate, or solvate thereof.

31. The method of claim 30 wherein the eflornithine is selected from the group consisting of: (i) a racemic mixture of D-eflornithine and L-eflornithine; (ii) D-eflornithine; and (iii) L-eflornithine.

32. The method of claim 21 wherein the eflornithine or derivative or analog thereof is a derivative or analog of eflornithine selected from the group consisting of:
(a) an analog of eflornithine of Formula (III): 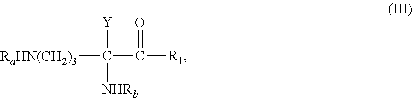
wherein:
(1) Y is FCH₂—, F₂CH—, or F₃C—;
(2) Rₐ and R_{b} are, independently, hydrogen, (C₁-C₄) alkylcarbonyl, or a group of Formula (III(a)) 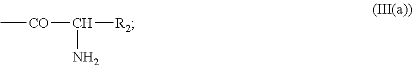
wherein, in Formula (III(a)), R₂ is hydrogen, (C₁-C₄) alkyl, benzyl, or p-hydroxybenzyl;
(3) R₁ is hydroxyl, (C₁-C₈) alkoxy, —NR₄R₅, wherein R₄ and R₅ are independently hydrogen, (C₁-C₄) alkyl, or a group of Formula (III(b)) 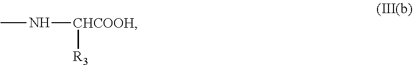
wherein, in Formula (III(b), R₃ is hydrogen, C₁-C₄) alkyl, or p-hydroxybenzyl;
(b) an analog of eflornithine of Formula (IV) or (V): 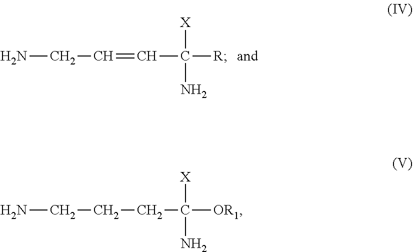
wherein:
(1) X is —CHF₂ or —CH₂F;
(2) R is hydrogen or —COR₁; and
(3) R₁ is —OH or (C₁-C₆) alkoxy;
(c) a water-soluble salt of eflornithine with a polycation selected from the group consisting of a polycationic carbohydrate, a polyaminoacid, a polyamine, a polypeptide, a basic polymer, or a quaternary ammonium compound;
(d) a conjugate in which a first moiety that is eflornithine or a derivative or analog of eflornithine is covalently linked to a second moiety that is a non-steroidal anti-inflammatory drug; and
(e) a copolymer of formula A-B-C or a pharmaceutically acceptable salt thereof, wherein A comprises a water soluble polymer; B comprises a matrix metalloprotease (MMP)-cleavable polypeptide; C is eflornithine; and A is connected to B at a first end through a first covalent bond or a first linking moiety and B is connected to C at a second end through a second covalent bond or a second linking moiety, and wherein the co-polymer is not crosslinked.

33. The method of claim 21 wherein the eflornithine or derivative or analog thereof is administered orally or by injection.

34. The method of claim 21 wherein the eflornithine or derivative or analog thereof is administered together with or adjuvant to radiotherapy.

35. The method of claim 21 wherein the malignancy is characterized by one or more of the following characteristics:
(a) the malignancy was previously treated with radiation therapy and adjuvant alkylator therapy and is a recurrent or refractory malignancy;
(b) the malignancy has a mutation in one or more genes selected from the group consisting of IDH1, IDH2, TP53, PTEN, ATRX, BRAF, CDKEN2A, SMARCA4, and PIK3;
(c) the malignancy has the promoter for MGMT methylated; and
(d) the malignancy has a mutation in at least one other gene that affects proliferation, survival, or resistance to chemotherapy.

36. The method of claim 21 wherein the eflornithine or derivative or analog thereof is administered together with an additional agent selected from the group consisting of:
(a) an inhibitor of polyamine transport;
(b) a polyamine analog;
(c) an S-adenosylmethionine decarboxylase inhibitor;
(d) an agent selected from the group consisting of: (1) a retinoid; (2) a syrbactin compound; (3) a cyclooxygenase-2 inhibitor; (4) a non-steroidal anti-inflammatory agent; (5) castanospermine or castanospermine esters; (6) an aziridinyl putrescine compound; (7) an interferon; (8) an aryl substituted xylopyranoside derivative; (9) an agent that reduces blood glutamate levels and enhances brain to blood glutamate efflux; (10) chitosan or chitosan derivatives and analogs; (11) 2,4-disulfonyl phenyl tert-butyl nitrone; (12) 3-(4-amino-1-oxo-1,3-dihydro-isoindol-2-yl)-piperidine-2,6-dione; (13) thalidomide; (14) N-2-pyridinyl-2-pyridinecarbothioamide; (15) cambendazole; and (16) an inhibitor of histone demethylase; and
(e) an agent that increases the ability of the eflornithine or derivative or analog thereof to pass through the blood-brain barrier.

37. The method of claim 21 wherein the eflornithine or derivative or analog of eflornithine is administered in a quantity sufficient to modulate an immune response to the malignancy.

38. The method of claim 37 wherein the eflornithine or derivative or analog of eflornithine is used together with one or more of: (1) passive immunotherapy with ex vivo activation of immune cells; (2) immunotherapy with chimeric antigen receptors; (3) ex vivo loading of dendritic cells with tumor antigens followed by reintroduction of the dendritic cells loaded with tumor antigens; (4) use of a vaccine targeting the IL-13 zetakine (IL13Ra2); (5) use of in situ gene therapy with Ad-Flt3L and Ad-Tk; (6) use of cancer stem cell antigens for vaccination; (7) use of macrophages loaded with gold-coated nanoshells in connection with photothermal therapy; (8) use of peripheral blood mononuclear cells that are collected and genetically modified to express the membrane-tethered IL-13 cytokine chimeric T cell receptor targeting the IL-13 receptor a2 (IL13Rα2); and (9) use of vaccine therapy with autologous dendritic cells.

39. The method of claim 21 wherein the eflornithine or derivative or analog thereof is administered together with a therapeutically effective quantity of one or more immunomodulatory agents used for the treatment of the malignancy.

40. A pharmaceutical composition for the treatment of glioma comprising:
(a) a therapeutically effective quantity of eflornithine or a derivative or analog thereof, wherein the therapeutically effective quantity of eflornithine or the derivative or analog thereof reduces the rate of mutation of hypermutation of the glioma to reduce the progression or grade of malignancy of the glioma caused by alkylating therapy exposure; and
(b) a pharmaceutically acceptable excipient.

41. The pharmaceutical composition of claim 40 wherein the reduction of the rate of hypermutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16.

42. The pharmaceutical composition of claim 41 wherein the reduction of the rate of hypermutation is a reduction of the rate of mutation of at least one gene selected from the group consisting of H1FNT, RFX1, DHX36, MBD2, FRK, PEG3, NDC80, ABCA6, DNLZ, MUC12, PDCD6IP, and GSC2.

43. The pharmaceutical composition of claim 40 wherein the composition is formulated for treatment of a subject with glioma, wherein the subject with glioma is selected from the group consisting of:
(i) a subject being currently treated with an alkylating agent;
(ii) a subject that had been previously treated with an alkylating agent;
(iii) a subject being currently treated with a platinum-containing antineoplastic agent that damages DNA;
(iv) a subject that had been previously treated with a platinum-containing antineoplastic agent that damages DNA; and
(v) a subject being currently or recently treated with radiotherapy.

44. The pharmaceutical composition of claim 40 wherein the eflornithine or derivative or analog thereof is selected from the group consisting of eflornithine and a pharmaceutically acceptable salt form, hydrate, or solvate thereof.

45. The pharmaceutical composition of claim 44 wherein the eflornithine is selected from the group consisting of: (i) a racemic mixture of D-eflornithine and L-eflornithine; (ii) D-eflornithine; and (iii) L-eflornithine.

46. The pharmaceutical composition of claim 41 wherein the eflornithine or derivative or analog thereof is a derivative or analog of eflornithine selected from the group consisting of:
(a) an analog of eflornithine of Formula (III): 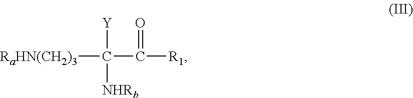
wherein:
(1) Y is FCH₂—, F₂CH—, or F₃C—;
(2) Rₐ and R_{b} are, independently, hydrogen, (C₁-C₄) alkylcarbonyl, or a group of Formula (III(a)) 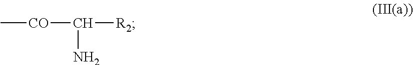
wherein, in Formula (III(a)), R₂ is hydrogen, (C₁-C₄) alkyl, benzyl, or p-hydroxybenzyl;
(3) R₁ is hydroxyl, (C₁-C₈) alkoxy, —NR₄R₅, wherein R₄ and R₅ are independently hydrogen, (C₁-C₄) alkyl, or a group of Formula (III(b)) 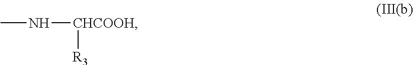
wherein, in Formula (III(b), R₃ is hydrogen, C₁-C₄) alkyl, or p-hydroxybenzyl;
(b) an analog of eflornithine of Formula (IV) or (V): 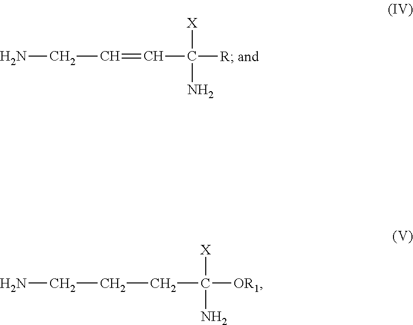
wherein:
(1) X is —CHF₂ or —CH₂F;
(2) R is hydrogen or —COR₁; and
(3) R₁ is —OH or (C₁-C₆) alkoxy;
(c) a water-soluble salt of eflornithine with a polycation selected from the group consisting of a polycationic carbohydrate, a polyaminoacid, a polyamine, a polypeptide, a basic polymer, or a quaternary ammonium compound;
(d) a conjugate in which a first moiety that is eflornithine or a derivative or analog of eflornithine is covalently linked to a second moiety that is a non-steroidal anti-inflammatory drug; and
(e) a copolymer of formula A-B-C or a pharmaceutically acceptable salt thereof, wherein A comprises a water soluble polymer; B comprises a matrix metalloprotease (MMP)-cleavable polypeptide; C is eflornithine; and A is connected to B at a first end through a first covalent bond or a first linking moiety and B is connected to C at a second end through a second covalent bond or a second linking moiety, and wherein the co-polymer is not crosslinked.

47. The pharmaceutical composition of claim 40 wherein the composition is formulated for oral administration or administration by injection.

48. The pharmaceutical composition of claim 47 wherein the composition is formulated for administration by injection, wherein the administration by injection is intravenous injection, intraperitoneal injection, subcutaneous injection, or intramuscular injection.

49. The pharmaceutical composition of claim 40 wherein the composition is formulated for systemic administration.

50. The pharmaceutical composition of claim 40 wherein the composition is formulated for localized administration.

51. The pharmaceutical composition of claim 40 wherein the composition further comprises a therapeutically effective quantity of one or more conventional antineoplastic agents used for the treatment of glioma, wherein the one or more conventional antineoplastic agents used for the treatment of glioma are selected from the group consisting of alkylating agents, platinum-containing agents that damage DNA, antimetabolites, anti-angiogenic agents, EGFR inhibitors, topoisomerase inhibitors, and tyrosine kinase inhibitors.

52. The pharmaceutical composition of claim 40 wherein the pharmaceutical composition further comprises a therapeutically effective quantity of an additional agent selected from the group consisting of:
(i) an inhibitor of polyamine transport;
(ii) a polyamine analog;
(iii) an S-adenosylmethionine decarboxylase inhibitor;
(iv) an agent selected from the group consisting of: (1) a retinoid; (2) a syrbactin compound; (3) a cyclooxygenase-2 inhibitor; (4) a non-steroidal anti-inflammatory agent; (5) castanospermine or castanospermine esters; (6) an aziridinyl putrescine compound; (7) an interferon; (8) an aryl substituted xylopyranoside derivative; (9) an agent that reduces blood glutamate levels and enhances brain to blood glutamate efflux; (10) chitosan or chitosan derivatives and analogs; (11) 2,4-disulfonyl phenyl tert-butyl nitrone; (12) 3-(4-amino-1-oxo-1,3-dihydro-isoindol-2-yl)-piperidine-2,6-dione; (13) thalidomide; (14) N-2-pyridinyl-2-pyridinecarbothioamide; (15) cambendazole; and (16) an inhibitor of histone demethylase; and
(v) an agent that increases the ability of the eflornithine or derivative or analog thereof to pass through the blood-brain barrier.

53. The pharmaceutical composition of claim 40 wherein the pharmaceutically acceptable excipient is selected from the group consisting of:
(i) a liquid carrier;
(ii) an isotonic agent;
(iii) a wetting or emulsifying agent;
(iv) a preservative;
(v) a buffer;
(vi) an acidifying agent;
(vii) an antioxidant;
(viii) an alkalinizing agent;
(ix) a carrying agent;
(x) a chelating agent;
(xi) a coloring agent;
(xii) a complexing agent;
(xiii) a solvent;
(xiv) a suspending and/or viscosity-increasing agent;
(xv) a flavor, perfume, or sweetening agent;
(xvi) an oil;
(xvii) a penetration enhancer;
(xviii) a polymer;
(xix) a stiffening agent;
(xx) a protein;
(xxi) a carbohydrate;
(xxii) a bulking agent; and
(xxiii) a lubricating agent.

54. A pharmaceutical composition for the treatment of a malignancy selected from the group consisting of a non-glioma central nervous system malignancy, colorectal cancer, leukemia, lymphoma, pancreatic cancer, liver cancer, and stomach cancer comprising:
(a) a therapeutically effective quantity of eflornithine or a derivative or analog thereof, wherein the therapeutically effective quantity of eflornithine or the derivative or analog thereof reduces the rate of hypermutation of the malignancy to reduce the progression or grade of the malignancy caused by alkylating therapy exposure; and
(b) a pharmaceutically acceptable excipient.

55. The pharmaceutical composition of claim 54 wherein the reduction of the rate of hypermutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16.

56. The pharmaceutical composition of claim 55 wherein the reduction of the rate of hypermutation is a reduction of the rate of mutation of at least one gene selected from the group consisting of H1FNT, RFX1, DHX36, MBD2, FRK, PEG3, NDC80, ABCA6, DNLZ, MUC12, PDCD6IP, and GSC2.

57. The pharmaceutical composition of claim 54 wherein the composition is formulated for treatment of a subject with the malignancy, wherein the subject with the malignancy is selected from the group consisting of:
(i) a subject being currently treated with an alkylating agent;
(ii) a subject that had been previously treated with an alkylating agent;
(iii) a subject being currently treated with a platinum-containing antineoplastic agent that damages DNA;
(iv) a subject that had been previously treated with a platinum-containing antineoplastic agent that damages DNA; and
(v) a subject being currently or recently treated with radiotherapy.

58. The pharmaceutical composition of claim 54 wherein the eflornithine or derivative or analog thereof is selected from the group consisting of eflornithine and a pharmaceutically acceptable salt form, hydrate, or solvate thereof.

59. The pharmaceutical composition of claim 58 wherein the eflornithine is selected from the group consisting of: (i) a racemic mixture of D-eflornithine and L-eflornithine; (ii) D-eflornithine; and (iii) L-eflornithine.

60. The pharmaceutical composition of claim 54 wherein the eflornithine or derivative or analog thereof is a derivative or analog of eflornithine selected from the group consisting of:
(a) an analog of eflornithine of Formula (III): 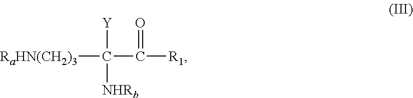
wherein:
(1) Y is FCH₂—, F₂CH—, or F₃C—;
(2) Rₐ and R_{b} are, independently, hydrogen, (C₁-C₄) alkylcarbonyl, or a group of Formula (III(a)) 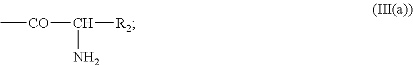
wherein, in Formula (III(a)), R₂ is hydrogen, (C₁-C₄) alkyl, benzyl, or p-hydroxybenzyl;
(3) R₁ is hydroxyl, (C₁-C₈) alkoxy, —NR₄R₅, wherein R₄ and R₅ are independently hydrogen, (C₁-C₄) alkyl, or a group of Formula (III(b)) 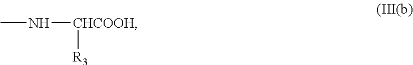
wherein, in Formula (III(b), R₃ is hydrogen, C₁-C₄) alkyl, or p-hydroxybenzyl;
(b) an analog of eflornithine of Formula (IV) or (V):
wherein: 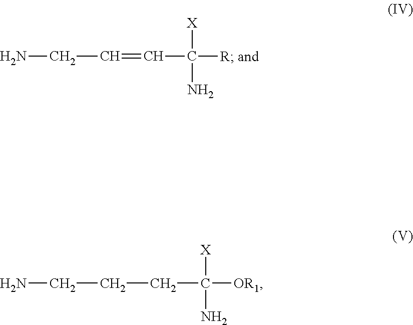
(1) X is —CHF₂ or —CH₂F;
(2) R is hydrogen or —COR₁; and
(3) R₁ is —OH or (C₁-C₆) alkoxy;
(c) a water-soluble salt of eflornithine with a polycation selected from the group consisting of a polycationic carbohydrate, a polyaminoacid, a polyamine, a polypeptide, a basic polymer, or a quaternary ammonium compound;
(d) a conjugate in which a first moiety that is eflornithine or a derivative or analog of eflornithine is covalently linked to a second moiety that is a non-steroidal anti-inflammatory drug; and
(e) a copolymer of formula A-B-C or a pharmaceutically acceptable salt thereof, wherein A comprises a water soluble polymer; B comprises a matrix metalloprotease (MMP)-cleavable polypeptide; C is eflornithine; and A is connected to B at a first end through a first covalent bond or a first linking moiety and B is connected to C at a second end through a second covalent bond or a second linking moiety, and wherein the co-polymer is not crosslinked.

61. The pharmaceutical composition of claim 54 wherein the composition is formulated for oral administration or administration by injection.

62. The pharmaceutical composition of claim 61 wherein the composition is formulated for administration by injection, wherein the administration by injection is intravenous injection, intraperitoneal injection, subcutaneous injection, or intramuscular injection.

63. The pharmaceutical composition of claim 54 wherein the composition is formulated for systemic administration.

64. The pharmaceutical composition of claim 54 wherein the composition is formulated for localized administration.

65. The pharmaceutical composition of claim 54 wherein the composition further comprises a therapeutically effective quantity of one or more conventional antineoplastic agents used for the treatment of the malignancy, wherein the one or more conventional antineoplastic agents used for the treatment of the malignancy are selected from the group consisting of alkylating agents, platinum-containing agents that damage DNA, antimetabolites, anti-angiogenic agents, EGFR inhibitors, topoisomerase inhibitors, and tyrosine kinase inhibitors.

66. The pharmaceutical composition of claim 54 wherein the pharmaceutical composition further comprises a therapeutically effective quantity of an additional agent selected from the group consisting of:
(i) an inhibitor of polyamine transport;
(ii) a polyamine analog;
(iii) an S-adenosylmethionine decarboxylase inhibitor;
(iv) an agent selected from the group consisting of: (1) a retinoid; (2) a syrbactin compound; (3) a cyclooxygenase-2 inhibitor; (4) a non-steroidal anti-inflammatory agent; (5) castanospermine or castanospermine esters; (6) an aziridinyl putrescine compound; (7) an interferon; (8) an aryl substituted xylopyranoside derivative; (9) an agent that reduces blood glutamate levels and enhances brain to blood glutamate efflux; (10) chitosan or chitosan derivatives and analogs; (11) 2,4-disulfonyl phenyl tert-butyl nitrone; (12) 3-(4-amino-1-oxo-1,3-dihydro-isoindol-2-yl)-piperidine-2,6-dione; (13) thalidomide; (14) N-2-pyridinyl-2-pyridinecarbothioamide; (15) cambendazole; and (16) an inhibitor of histone demethylase; and
(v) an agent that increases the ability of the eflornithine or derivative or analog thereof to pass through the blood-brain barrier.

67. The pharmaceutical composition of claim 54 wherein the pharmaceutically acceptable excipient is selected from the group consisting of:
(i) a liquid carrier;
(ii) an isotonic agent;
(iii) a wetting or emulsifying agent;
(iv) a preservative;
(v) a buffer;
(vi) an acidifying agent;
(vii) an antioxidant;
(viii) an alkalinizing agent;
(ix) a carrying agent;
(x) a chelating agent;
(xi) a coloring agent;
(xii) a complexing agent;
(xiii) a solvent;
(xiv) a suspending and/or viscosity-increasing agent;
(xv) a flavor, perfume, or sweetening agent;
(xvi) an oil;
(xvii) a penetration enhancer;
(xviii) a polymer;
(xix) a stiffening agent;
(xx) a protein;
(xxi) a carbohydrate;
(xxii) a bulking agent; and
(xxiii) a lubricating agent.

68. A pharmaceutical composition for the treatment of glioma comprising:
(a) a therapeutically effective quantity of eflornithine or a derivative or analog thereof, wherein the therapeutically effective quantity of eflornithine or the derivative or analog thereof reduces the rate of hypermutation of the glioma to reduce the progression or grade of malignancy of the glioma caused by alkylating therapy exposure;
(b) a therapeutically effective quantity of an alkylating agent; and
(c) a pharmaceutically acceptable excipient.

69. The pharmaceutical composition of claim 68 wherein the reduction of the rate of hypermutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16.

70. The pharmaceutical composition of claim 69 wherein the reduction of the rate of hypermutation is a reduction of the rate of mutation of at least one gene selected from the group consisting of H1FNT, RFX1, DHX36, MBD2, FRK, PEG3, NDC80, ABCA6, DNLZ, MUC12, PDCD6IP, and GSC2.

71. The pharmaceutical composition of claim 68 wherein the composition is formulated for treatment of a subject with glioma, wherein the subject with glioma is selected from the group consisting of:
(i) a subject being currently treated with an alkylating agent;
(ii) a subject that had been previously treated with an alkylating agent;
(iii) a subject being currently treated with a platinum-containing antineoplastic agent that damages DNA;
(iv) a subject that had been previously treated with a platinum-containing antineoplastic agent that damages DNA; and
(v) a subject being currently or recently treated with radiotherapy.

72. The pharmaceutical composition of claim 68 wherein the eflornithine or derivative or analog thereof is selected from the group consisting of eflornithine and a pharmaceutically acceptable salt form, hydrate, or solvate thereof.

73. The pharmaceutical composition of claim 72 wherein the eflornithine is selected from the group consisting of: (i) a racemic mixture of D-eflornithine and L-eflornithine; (ii) D-eflornithine; and (iii) L-eflornithine.

74. The pharmaceutical composition of claim 68 wherein the eflornithine or derivative or analog thereof is a derivative or analog of eflornithine selected from the group consisting of:
(a) an analog of eflornithine of Formula (III): 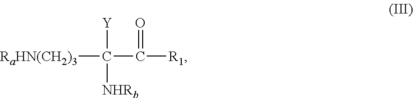
wherein:
(1) Y is FCH₂—, F₂CH—, or F₃C—;
(2) Rₐ and R_{b} are, independently, hydrogen, (C₁-C₄) alkylcarbonyl, or a group of Formula (III(a)) 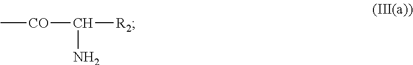
wherein, in Formula (III(a)), R₂ is hydrogen, (C₁-C₄) alkyl, benzyl, or p-hydroxybenzyl;
(3) R₁ is hydroxyl, (C₁-C₈) alkoxy, —NR₄R₅, wherein R₄ and R₅ are independently hydrogen, (C₁-C₄) alkyl, or a group of Formula (III(b)) 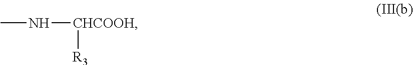
wherein, in Formula (III(b), R₃ is hydrogen, C₁-C₄) alkyl, or p-hydroxybenzyl;
(b) an analog of eflornithine of Formula (IV) or (V): 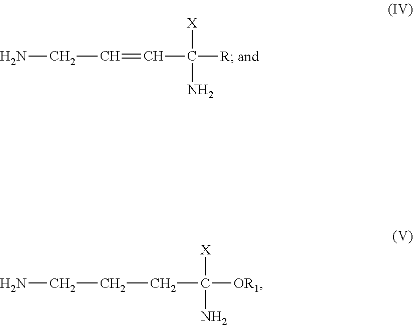
wherein:
(1) X is —CHF₂ or —CH₂F;
(2) R is hydrogen or —COR₁; and
(3) R₁ is —OH or (C₁-C₆) alkoxy;
(c) a water-soluble salt of eflornithine with a polycation selected from the group consisting of a polycationic carbohydrate, a polyaminoacid, a polyamine, a polypeptide, a basic polymer, or a quaternary ammonium compound;
(d) a conjugate in which a first moiety that is eflornithine or a derivative or analog of eflornithine is covalently linked to a second moiety that is a non-steroidal anti-inflammatory drug; and
(e) a copolymer of formula A-B-C or a pharmaceutically acceptable salt thereof, wherein A comprises a water soluble polymer; B comprises a matrix metalloprotease (MMP)-cleavable polypeptide; C is eflornithine; and A is connected to B at a first end through a first covalent bond or a first linking moiety and B is connected to C at a second end through a second covalent bond or a second linking moiety, and wherein the co-polymer is not crosslinked.

75. The pharmaceutical composition of claim 68 wherein the composition is formulated for oral administration or administration by injection.

76. The pharmaceutical composition of claim 75 wherein the composition is formulated for administration by injection, wherein the administration by injection is intravenous injection, intraperitoneal injection, subcutaneous injection, or intramuscular injection.

77. The pharmaceutical composition of claim 68 wherein the composition is formulated for systemic administration.

78. The pharmaceutical composition of claim 68 wherein the composition is formulated for localized administration.

79. The pharmaceutical composition of claim 68 wherein the composition further comprises a therapeutically effective quantity of one or more conventional antineoplastic agents used for the treatment of glioma, wherein the one or more conventional antineoplastic agents used for the treatment of glioma are selected from the group consisting of platinum-containing agents that damage DNA, antimetabolites, anti-angiogenic agents, EGFR inhibitors, topoisomerase inhibitors, and tyrosine kinase inhibitors.

80. The pharmaceutical composition of claim 68 wherein the pharmaceutical composition further comprises a therapeutically effective quantity of an additional agent selected from the group consisting of:
(i) an inhibitor of polyamine transport;
(ii) a polyamine analog;
(iii) an S-adenosylmethionine decarboxylase inhibitor;
(iv) an agent selected from the group consisting of: (1) a retinoid; (2) a syrbactin compound; (3) a cyclooxygenase-2 inhibitor; (4) a non-steroidal anti-inflammatory agent; (5) castanospermine or castanospermine esters; (6) an aziridinyl putrescine compound; (7) an interferon; (8) an aryl substituted xylopyranoside derivative; (9) an agent that reduces blood glutamate levels and enhances brain to blood glutamate efflux; (10) chitosan or chitosan derivatives and analogs; (11) 2,4-disulfonyl phenyl tert-butyl nitrone; (12) 3-(4-amino-1-oxo-1,3-dihydro-isoindol-2-yl)-piperidine-2,6-dione; (13) thalidomide; (14) N-2-pyridinyl-2-pyridinecarbothioamide; (15) cambendazole; and (16) an inhibitor of histone demethylase; and
(v) an agent that increases the ability of the eflornithine or derivative or analog thereof to pass through the blood-brain barrier.

81. The pharmaceutical composition of claim 68 wherein the alkylating agent is selected from the group consisting of temozolomide and lomustine.

82. The pharmaceutical composition of claim 68 wherein the alkylating agent is selected from the group consisting of:
(i) cyclophosphamide;
(ii) mechlorethamine;
(iii) uracil mustard;
(iv) melphalan;
(v) chlorambucil;
(vi) ifosfamide;
(vii) bendamustine;
(viii) carmustine;
(ix) streptozotocin;
(x) busulfan;
(xi) procarbazine;
(xii) dacarbazine;
(xiii) mitocarbazine;
(xiv) altretamine;
(xv) 6-methyluracil mustard;
(xvi) 6-ethyluracil mustard;
(xvii) 6-propyluracil mustard;
(xviii) 3-[1-methyl-4-[1-methyl-4-[1-methyl-4-[N1-[5-bis(2-chloroethyl)amino-2,4-(1H,3H)pyrimidinedione]acetylamino]-pyrrole-2-carboxamido]propionamidine hydrochloride;
(xix) 3-[1-methyl-4-[1-methyl-4-[1-methyl-4-[N1-[5-bis(2-chloroethyl)amino-2,4-(1H,3H)pyrimidinedione]propanoylamino]-pyrrole-2-carboxamido]propionamidine hydrochloride;
(xx) 3-[1-methyl-4-[1-methyl-4-[1-methyl-4-[N1-[5-bis(2-chloroethyl)amino-2,4-(1H,3H)pyrimidinedione]butanoylamino]-pyrrole-2-carboxamido]propionamidine hydrochloride;
(xxi) 3-[1-methyl-4-[1-methyl-4-[1-methyl-4-[N1-[5-bis(2-chloroethyl)amino-2,4-(1H,3H)pyrimidinedione]pentanoylamino]-pyrrole-2-carboxamido]propionamidine hydrochloride;
(xxii) 3-[1-methyl-4-[1-methyl-4-[1-methyl-4-[N1-[5-bis(2-chloroethyl)amino-2,4-(1H,3H)pyrimidinedione]hexanoylamino]-pyrrole-2-carboxamido]propionamidine hydrochloride;
(xxiii) 3-[1-methyl-4-[1-methyl-4-[1-methyl-4-[N1-[5-bis(2-chloroethyl)amino-2,4-(1H,3H)pyrimidinedione]heptanoylamino]-pyrrole-2-carboxamido]propionamidine hydrochloride;
(xxiv) estramustine;
(xxv) quinacrine mustard dihydrochloride;
(xxvi) spiromustine;
(xxvii) mustamine;
(xxviii) phenylalanine mustard;
(xxix) mannomustine;
(xxx) 5-((bis(2-chloroethyl)amino)methyl)-pyrimidine-2,4(1H,3H)-dione;
(xxxi) 5-((bis(2-chloroethyl)amino)methyl)-6-methylpyrimidine-2,4(1H,3H)-dione;
(xxxii) 5-((bis(2-chloroethyl)amino)methyl)-1-methylpyrimidine-2,4(1H,3H)-dione;
(xxxiii) 5-((bis(2-chloroethyl)amino)methyl)-1,3-dimethylpyrimidine-2,4(1H,3H)-dione;
(xxxiv) 5-((bis(2-chloroethyl)amino)methyl)-6-propylpyrimidine-2,4(1H, 3H)-dione;
(xxxv) 5-((bis(2-chloroethyl)amino)methyl)-6-methyl-2-thioxo-2,3-dihydropyrimidin-4(1H)-one;
(xxxvi) nitrouracil;
(xxxvii) 5,6-dihydro-5-nitrouracil;
(xxxviii) 5,6-dihydro-5-nitro-1-(4-nitrophenyl)uracil;
(xxxix) 5-nitro-1-(4-nitrophenyl)uracil;
(xl) 5,6-dihydro-5-nitro-1 (3-D-ribofuranuronic acid ethyl ester)uracil;
(xli) 5-nitro-1(β-D-ribofuranuronic acid ethyl ester)uracil;
(xlii) 5-nitrouracil N-oxide;
(xliii) prednimustine;
(xliv) nimustine;
(xlv) ranimustine;
(xlvi) fotemustine;
(xlvii) ribomustine;
(xlviii) cystemustine;
(xlix) 4-chlorouracil mustard;
(l) 4-cyanouracil mustard;
(li) 4-nitrouracil mustard;
(lii) dianhydrogalactitol;
(liii) diacetyldianhydrogalactitol; and
(liv) dibromodulcitol.

83. The pharmaceutical composition of claim 68 wherein the pharmaceutically acceptable excipient is selected from the group consisting of:
(i) a liquid carrier;
(ii) an isotonic agent;
(iii) a wetting or emulsifying agent;
(iv) a preservative;
(v) a buffer;
(vi) an acidifying agent;
(vii) an antioxidant;
(viii) an alkalinizing agent;
(ix) a carrying agent;
(x) a chelating agent;
(xi) a coloring agent;
(xii) a complexing agent;
(xiii) a solvent;
(xiv) a suspending and/or viscosity-increasing agent;
(xv) a flavor, perfume, or sweetening agent;
(xvi) an oil;
(xvii) a penetration enhancer;
(xviii) a polymer;
(xix) a stiffening agent;
(xx) a protein;
(xxi) a carbohydrate;
(xxii) a bulking agent; and
(xxiii) a lubricating agent.

84. A kit, comprising, separately packaged:
(a) a therapeutically effective quantity of eflornithine or a derivative or analog thereof, wherein the quantity of the eflornithine or the derivative or analog thereof is a therapeutically effective quantity for treatment of a glioma in order to reduce the rate of hypermutation of the glioma to reduce the progression or grade of malignancy of the glioma caused by alkylating therapy exposure;
(b) a therapeutically effective quantity of an alkylating agent for treatment of the glioma; and
(c) instructions for use of the kit.

85. The kit of claim 84 wherein the reduction of the rate of hypermutation is a reduction of the rate of mutation of at least one gene selected from the group consisting of MUC12, DNLZ, CACNA1B, LRP1B, PCDHG3, ICAM1, H1FNT, RFX1, DHX36, MBD2, TRBV10-1, FRK, RNF222, PEG3, CYP11B2, NDC80, AP3B1, ABCA6, ZRSR1, AC110781.3, ANKLE1, CROCC, GSC2, LCMT1, METTL1, PCNT, PDCD6IP, CYP39A1, RBMXL1, MSH1, TP53, ADAM32, GPR116, and MUC16.

86. The kit of claim 85 wherein the reduction of the rate of hypermutation is a reduction of the rate of mutation of at least one gene selected from the group consisting of H1FNT, RFX1, DHX36, MBD2, FRK, PEG3, NDC80, ABCA6, DNLZ, MUC12, PDCD6IP, and GSC2.

87. The kit of claim 84 wherein the kit further comprises, separately packaged, a pharmaceutically acceptable excipient to be combined with the therapeutically effective quantity of the eflornithine or the derivative or analog thereof.

88. The kit of claim 87 wherein the pharmaceutically acceptable excipient to be combined with the therapeutically effective quantity of the eflornithine or the derivative thereof is selected from the group consisting of:
(i) a liquid carrier;
(ii) an isotonic agent;
(iii) a wetting or emulsifying agent;
(iv) a preservative;
(v) a buffer;
(vi) an acidifying agent;
(vii) an antioxidant;
(viii) an alkalinizing agent;
(ix) a carrying agent;
(x) a chelating agent;
(xi) a coloring agent;
(xii) a complexing agent;
(xiii) a solvent;
(xiv) a suspending and/or viscosity-increasing agent;
(xv) a flavor, perfume, or sweetening agent;
(xvi) an oil;
(xvii) a penetration enhancer;
(xviii) a polymer;
(xix) a stiffening agent;
(xx) a protein;
(xxi) a carbohydrate;
(xxii) a bulking agent; and
(xxiii) a lubricating agent.

88. The kit of claim 84 wherein the kit further comprises, separately packaged, a pharmaceutically acceptable excipient to be combined with the therapeutically effective quantity of the alkylating agent.

89. The kit of claim 88 wherein the pharmaceutically acceptable excipient is selected from the group consisting of:
(i) a liquid carrier;
(ii) an isotonic agent;
(iii) a wetting or emulsifying agent;
(iv) a preservative;
(v) a buffer;
(vi) an acidifying agent;
(vii) an antioxidant;
(viii) an alkalinizing agent;
(ix) a carrying agent;
(x) a chelating agent;
(xi) a coloring agent;
(xii) a complexing agent;
(xiii) a solvent;
(xiv) a suspending and/or viscosity-increasing agent;
(xv) a flavor, perfume, or sweetening agent;
(xvi) an oil;
(xvii) a penetration enhancer;
(xviii) a polymer;
(xix) a stiffening agent;
(xx) a protein;
(xxi) a carbohydrate;
(xxii) a bulking agent; and
(xxiii) a lubricating agent.

90. The kit of claim 84 wherein the eflornithine or derivative or analog thereof is selected from the group consisting of eflornithine and a pharmaceutically acceptable salt form, hydrate, or solvate thereof.

91. The kit of claim 90 wherein the eflornithine is selected from the group consisting of: (i) a racemic mixture of D-eflornithine and L-eflornithine; (ii) D-eflornithine; and (iii) L-eflornithine.

92. The kit of claim 84 wherein the eflornithine or derivative or analog thereof is a derivative or analog of eflornithine selected from the group consisting of:
(a) an analog of eflornithine of Formula (III): 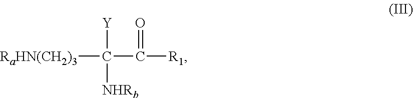
wherein:
(1) Y is FCH₂—, F₂CH—, or F₃C—;
(2) Rₐ and R_{b} are, independently, hydrogen, (C₁-C₄) alkylcarbonyl, or a group of Formula (III(a)) 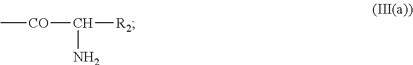
wherein, in Formula (III(a)), R₂ is hydrogen, (C₁-C₄) alkyl, benzyl, or p-hydroxybenzyl;
(3) R₁ is hydroxyl, (C₁-C₈) alkoxy, —NR₄R₅, wherein R₄ and R₅ are independently hydrogen, (C₁-C₄) alkyl, or a group of Formula (III(b)) 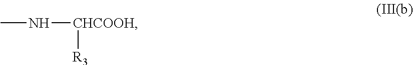
wherein, in Formula (III(b), R₃ is hydrogen, C₁-C₄) alkyl, or p-hydroxybenzyl;
(b) an analog of eflornithine of Formula (IV) or (V): 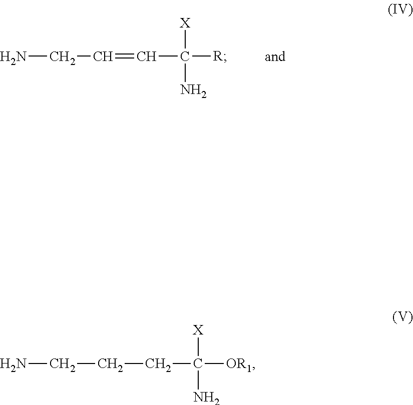
wherein:
(1) X is —CHF₂ or —CH₂F;
(2) R is hydrogen or —COR₁; and
(3) R₁ is —OH or (C₁-C₆) alkoxy;
(c) a water-soluble salt of eflornithine with a polycation selected from the group consisting of a polycationic carbohydrate, a polyaminoacid, a polyamine, a polypeptide, a basic polymer, or a quaternary ammonium compound;
(d) a conjugate in which a first moiety that is eflornithine or a derivative or analog of eflornithine is covalently linked to a second moiety that is a non-steroidal anti-inflammatory drug; and
(e) a copolymer of formula A-B-C or a pharmaceutically acceptable salt thereof, wherein A comprises a water soluble polymer; B comprises a matrix metalloprotease (MMP)-cleavable polypeptide; C is eflornithine; and A is connected to B at a first end through a first covalent bond or a first linking moiety and B is connected to C at a second end through a second covalent bond or a second linking moiety, and wherein the co-polymer is not crosslinked.

93. The kit of claim 84 wherein the kit is formulated such that the eflornithine or derivative or analog thereof is administered orally or by injection.

94. The kit of claim 84 wherein the alkylating agent is selected from the group consisting of temozolomide and lomustine.

95. The kit of claim 84 wherein the alkylating agent is selected from the group consisting of:
(i) cyclophosphamide;
(ii) mechlorethamine;
(iii) uracil mustard;
(iv) melphalan;
(v) chlorambucil;
(vi) ifosfamide;
(vii) bendamustine;
(viii) carmustine;
(ix) streptozotocin;
(x) busulfan;
(xi) procarbazine;
(xii) dacarbazine;
(xiii) mitocarbazine;
(xiv) altretamine;
(xv) 6-methyluracil mustard;
(xvi) 6-ethyluracil mustard;
(xvii) 6-propyluracil mustard;
(xviii) 3-[1-methyl-4-[1-methyl-4-[1-methyl-4-[N1-[5-bis(2-chloroethyl)amino-2,4-(1H,3H)pyrimidinedione]acetylamino]-pyrrole-2-carboxamido]propionamidine hydrochloride;
(xix) 3-[1-methyl-4-[1-methyl-4-[1-methyl-4-[N1-[5-bis(2-chloroethyl)amino-2,4-(1H,3H)pyrimidinedione]propanoylamino]-pyrrole-2-carboxamido]propionamidine hydrochloride;
(xx) 3-[1-methyl-4-[1-methyl-4-[1-methyl-4-[N1-[5-bis(2-chloroethyl)amino-2,4-(1H,3H)pyrimidinedione]butanoylamino]-pyrrole-2-carboxamido]propionamidine hydrochloride;
(xxi) 3-[1-methyl-4-[1-methyl-4-[1-methyl-4-[N1-[5-bis(2-chloroethyl)amino-2,4-(1H,3H)pyrimidinedione]pentanoylamino]-pyrrole-2-carboxamido]propionamidine hydrochloride;
(xxii) 3-[1-methyl-4-[1-methyl-4-[1-methyl-4-[N1-[5-bis(2-chloroethyl)amino-2,4-(1H,3H)pyrimidinedione]hexanoylamino]-pyrrole-2-carboxamido]propionamidine hydrochloride;
(xxiii) 3-[1-methyl-4-[1-methyl-4-[1-methyl-4-[N1-[5-bis(2-chloroethyl)amino-2,4-(1H,3H)pyrimidinedione]heptanoylamino]-pyrrole-2-carboxamido]propionamidine hydrochloride;
(xxiv) estramustine;
(xxv) quinacrine mustard dihydrochloride;
(xxvi) spiromustine;
(xxvii) mustamine;
(xxviii) phenylalanine mustard;
(xxix) mannomustine;
(xxx) 5-((bis(2-chloroethyl)amino)methyl)-pyrimidine-2,4(1H,3H)-dione;
(xxxi) 5-((bis(2-chloroethyl)amino)methyl)-6-methylpyrimidine-2,4(1H,3H)-dione;
(xxxii) 5-((bis(2-chloroethyl)amino)methyl)-1-methylpyrimidine-2,4(1H,3H)-dione;
(xxxiii) 5-((bis(2-chloroethyl)amino)methyl)-1,3-dimethylpyrimidine-2,4(1H,3H)-dione;
(xxxiv) 5-((bis(2-chloroethyl)amino)methyl)-6-propylpyrimidine-2,4(1H, 3H)-dione;
(xxxv) 5-((bis(2-chloroethyl)amino)methyl)-6-methyl-2-thioxo-2,3-dihydropyrimidin-4(1H)-one;
(xxxvi) nitrouracil;
(xxxvii) 5,6-dihydro-5-nitrouracil;
(xxxviii) 5,6-dihydro-5-nitro-1-(4-nitrophenyl)uracil;
(xxxix) 5-nitro-1-(4-nitrophenyl)uracil;
(xl) 5,6-dihydro-5-nitro-1(β-D-ribofuranuronic acid ethyl ester)uracil;
(xli) 5-nitro-1(β-D-ribofuranuronic acid ethyl ester)uracil;
(xlii) 5-nitrouracil N-oxide;
(xliii) prednimustine;
(xliv) nimustine;
(xlv) ranimustine;
(xlvi) fotemustine;
(xlvii) ribomustine;
(xlviii) cystemustine;
(xlix) 4-chlorouracil mustard;
(l) 4-cyanouracil mustard;
(li) 4-nitrouracil mustard;
(lii) dianhydrogalactitol;
(liii) diacetyldianhydrogalactitol; and
(liv) dibromodulcitol.
